(19) United States
(12) Patent Application Publication
(10) Pub. No.: US 2018/0318287 A1
(43) Pub. Date: Nov. 8, 2018

(54) Imidazo[4,5-c]quinolin-2-one Compounds and Their Use in Treating Cancer

(71) Applicant: ASTRAZENECA AB, Södertälje (SE)

(72) Inventors: Kurt Gordon PIKE, Cambridge (GB); Bernard Christophe BARLAAM, Cambridge (GB); Thomas Anthony HUNT, Cambridge (GB); Andrew John EATHERTON, Cambridge (GB)

(73) Assignee: ASTRAZENECA AB, Södertälje (SE)

(21) Appl. No.: 15/772,118

(22) PCT Filed: Nov. 2, 2016
(86) PCT No.: PCT/EP2016/076412

§ 371 Date: Apr. 30, 2018

(30) Foreign Application Priority Data
Nov. 3, 2015 (GB) 1519406.1

(51) Int. Cl. A61K 31/4745; A61P 35/00

(52) U.S. Cl. CPC A61K 31/4745; A61P 35/00; A61K 31/282; A61K 2300/00; A61K 2121/00

(57) ABSTRACT

The specification generally relates to compounds of Formula (I):

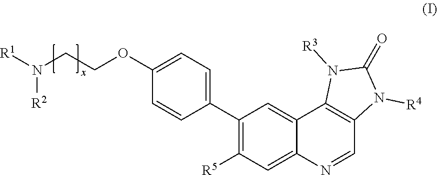

and pharmaceutically acceptable salts thereof, where x, R¹, R², R³, R⁴ and R⁵ have any of the meanings defined herein. The specification also relates to the use of compounds of Formula (I) and salts thereof to treat or prevent ATM mediated disease, including cancer. The specification further relates to pharmaceutical compositions comprising substituted imidazo[4,5-c]quinolin-2-one compounds and pharmaceutically acceptable salts thereof; kits comprising such compounds and salts; methods of manufacture of such compounds and salts; and intermediates useful in such manufacture.

### FIELD OF INVENTION

This specification relates to substituted imidazo[4,5-c]quinolin-2-one compounds and pharmaceutically acceptable salts thereof. These compounds and salts selectively modulate ataxia telangiectasia mutated (“ATM”) kinase, and the specification therefore also relates to the use of substituted imidazo[4,5-c]quinolin-2-one compounds and salts thereof to treat or prevent ATM mediated disease, including cancer. The specification further relates to pharmaceutical compositions comprising substituted imidazo[4,5-c]quinolin-2-one compounds and pharmaceutically acceptable salts thereof; kits comprising such compounds and salts; methods of manufacture of such compounds and salts; and intermediates useful in such manufacture.

### BACKGROUND

ATM kinase is a serine threonine kinase originally identified as the product of the gene mutated in ataxia telangiectasia. Ataxia telangiectasia is located on human chromosome 11q22-23 and codes for a large protein of about 350 kDa, which is characterized by the presence of a phosphatidylinositol (“PI”) 3-kinase-like serine/threonine kinase domain flanked by FRAP-ATM-TRRAP and FATC domains which modulate ATM kinase activity and function. ATM kinase has been identified as a major player of the DNA damage response elicited by double strand breaks. It primarily functions in S/G2/M cell cycle transitions and at collapsed replication forks to initiate cell cycle checkpoints, chromatin modification, HR repair and pro-survival signalling cascades in order to maintain cell integrity after DNA damage (Lavin, M. F.; Rev. Mol. Cell Biol. 2008, 759-769).

ATM kinase signalling can be broadly divided into two categories: a canonical pathway, which signals together with the Mre11-Rad50-NBS1 complex from double strand breaks and activates the DNA damage checkpoint, and several non-canonical modes of activation, which are activated by other forms of cellular stress (Cremona et al., Oncogene 2013, 3351-3360).

ATM kinase is rapidly and robustly activated in response to double strand breaks and is reportedly able to phosphorylate in excess of 800 substrates (Matsuoka et al., Science 2007, 1160-1166), coordinating multiple stress response pathways (Kurz and Lees Miller, DNA Repair 2004, 889-900.). ATM kinase is present predominantly in the nucleus of the cell in an inactive homodimeric form but autophosphorylates itself on Ser1981 upon sensing a DNA double strand break (canonical pathway), leading to dissociation to a monomer with full kinase activity (Bakkenist et al., Nature 2003, 499-506). This is a critical activation event, and ATM phospho-Ser1981 is therefore both a direct pharmacodynamic and patient selection biomarker for tumour pathway dependency.

ATM kinase responds to direct double strand breaks caused by common anti-cancer treatments such as ionising radiation and topoisomerase-II inhibitors (doxorubicin, etoposide) but also to topoisomerase-I inhibitors (for example irinotecan and topotecan) via single strand break to double strand break conversion during replication. ATM kinase inhibition can potentiate the activity of any these agents, and as a result ATM kinase inhibitors are expected to be of use in the treatment of cancer.

CN102372711A reports certain imidazo[4,5-c]quinolin-2-one compounds which are mentioned to be dual inhibitors of PI 3-kinase α and mammalian target of rapamycin (“mTOR”) kinase. Among the compounds reported in CN102372711A are the following:

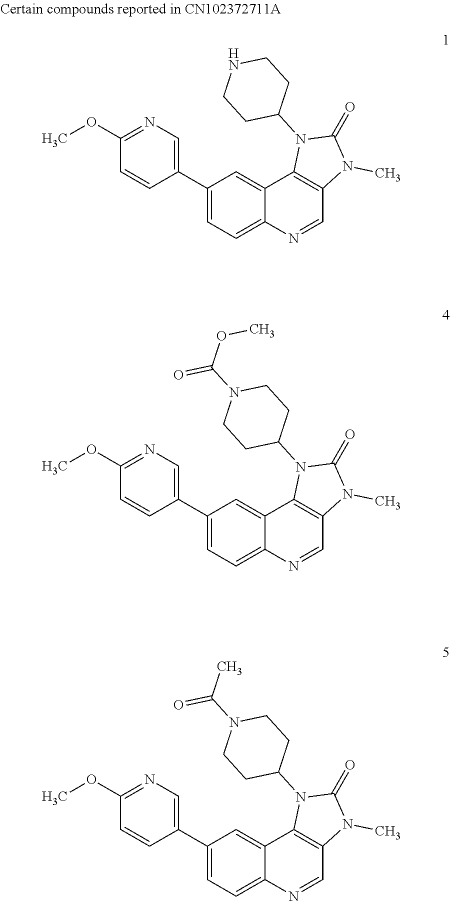

CN 102399218A reports certain imidazo[4,5-c]quinolin-2-one compounds which are mentioned to be PI 3-kinase α inhibitors. Among the compounds reported in CN102399218A are the following:

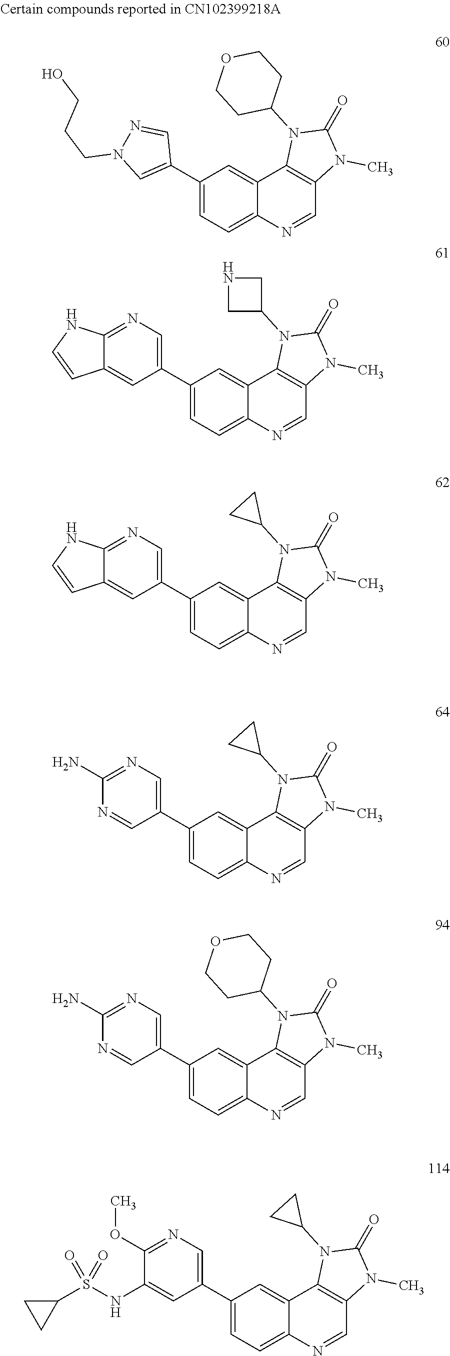

While the compounds or CN102372711A and CN102399218A are reported to possess activity against PI 3-kinase α and in some cases mTOR kinase, there remains a need to develop new compounds that are more effective against different kinase enzymes, such as ATM kinase. There further exists a need for new compounds which act against certain kinase enzymes, like ATM kinase, in a highly selective fashion (i.e. by modulating ATM more effectively than other biological targets).

As demonstrated elsewhere in the specification (for example in the cell based assays described in the experimental section), the compounds of the present specification generally possess very potent ATM kinase inhibitory activity, but much less potent activity against other tyrosine kinase enzymes, such as PI 3-kinase α, mTOR kinase and ataxia telangiectasia and Rad3-related protein (“ATR”) kinase. As such, the compounds of the present specification not only inhibit ATM kinase, but can be considered to be highly selective inhibitors of ATM kinase.

As a result of their highly selective nature, the compounds of the present specification are expected to be particularly useful in the treatment of diseases in which ATM kinase is implicated (for example, in the treatment of cancer), but where it is desirable to minimise off-target effects or toxicity that might arise due to the inhibition of other tyrosine kinase enzymes, such as class PI 3-kinase α, mTOR kinase and ATR kinase.

### SUMMARY OF INVENTION

Briefly, this specification describes, in part, a compound of Formula (I):

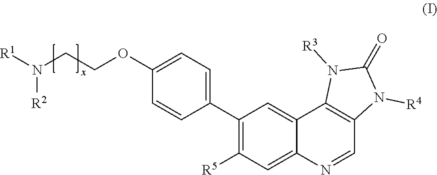

or a pharmaceutically acceptable salt thereof, where:

R¹ is methyl;

R² is hydro or methyl; or

R¹ and R² together with the nitrogen atom to which they are bonded form an azetidinyl, pyrrolidinyl or piperidinyl ring;

x is 1 or 2;

R³ is:
C₄-C₆ cycloalkyl optionally substituted with one methoxy group,
isopropyl,
tetrahydrofuranyl, or
tetrahydropyranyl;

R⁴ is hydro or methyl; and

R⁵ is hydro or fluoro.

This specification also describes, in part, a pharmaceutical composition which comprises a compound of Formula (I), or a pharmaceutically acceptable salt thereof, and at least one pharmaceutically acceptable excipient.

This specification also describes, in part, a compound of Formula (I), or a pharmaceutically acceptable salt thereof, for use in therapy.

This specification also describes, in part, a compound of Formula (I), or a pharmaceutically acceptable salt thereof, for use in the treatment of cancer.

This specification also describes, in part, the use of a compound of Formula (I), or a pharmaceutically acceptable salt thereof, in the manufacture of a medicament for the treatment of cancer.

This specification also describes, in part, a method for treating cancer in a warm blooded animal in need of such treatment, which comprises administering to said warm-blooded animal a therapeutically effective amount of a compound of Formula (I), or a pharmaceutically acceptable salt thereof.

### ILLUSTRATIVE EMBODIMENTS

Many embodiments of the invention are detailed throughout the specification and will be apparent to a reader skilled in the art. The invention is not to be interpreted as being limited to any particular embodiment(s) thereof.

In the first embodiment there is provided a compound of Formula (I):

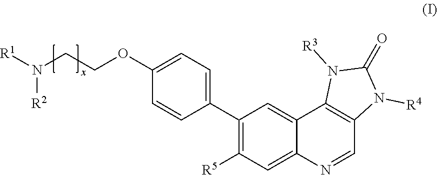

or a pharmaceutically acceptable salt thereof, where:

R¹ is methyl;

R² is hydro or methyl; or

R¹ and R² together with the nitrogen atom to which they are bonded form an azetidinyl, pyrrolidinyl or piperidinyl ring;

x is 1 or 2;

R³ is:
C₄-C₆ cycloalkyl optionally substituted with one methoxy group,
isopropyl,
tetrahydrofuranyl, or
tetrahydropyranyl;

R⁴ is hydro or methyl; and

R⁵ is hydro or fluoro.

A “hydro” group is equivalent to a hydrogen atom. Atoms with a hydro group attached to them can be regarded as unsubstituted.

“C₄-C₆ cycloalkyl” means a non-aromatic carbocyclic ring comprising 4 to 6 ring carbon atoms and no ring heteroatoms. C₄-C₆ cycloalkyl includes cyclobutyl, cyclopentyl, and cyclohexyl groups.

Where the term “optionally” is used, it is intended that the subsequent feature may or may not occur. As such, use of the term “optionally” includes instances where the feature is present, and also instances where the feature is not present. For example, a “C₄-C₆ cycloalkyl optionally substituted with one methoxy group” includes cyclobutyl, cyclopentyl and cyclohexyl groups with or without the specified substituents.

Where it is mentioned that “R¹ and R² together with the nitrogen atom to which they are bonded form an azetidinyl, pyrrolidinyl or piperidinyl ring”, this means the R¹ and R² groups are joined via a carbon-carbon covalent bond to form an unsubstituted alkylene chain of the appropriate length to form the corresponding ring. For example, when R¹ and R² together with the nitrogen atom to which they are bonded form a pyrrolidinyl ring, R¹ and R² together represent an unsubstituted butylene chain which is attached to the relevant nitrogen atom in Formula (I) at both terminal carbons.

The term “pharmaceutically acceptable” is used to specify that an object (for example a salt, dosage form or excipient) is suitable for use in patients. An example list of pharmaceutically acceptable salts can be found in the Handbook of Pharmaceutical Salts: Properties, Selection and Use, P. H. Stahl and C. G. Wermuth, editors, Weinheim/zürich:Wiley-VCH/VHCA, 2002. A suitable pharmaceutically acceptable salt of a compound of Formula (I) is, for example, an acid-addition salt. An acid addition salt of a compound of Formula (I) may be formed by bringing the compound into contact with a suitable inorganic or organic acid under conditions known to the skilled person. An acid addition salt may for example be formed using an inorganic acid selected from hydrochloric acid, hydrobromic acid, sulphuric acid and phosphoric acid. An acid addition salt may also be formed using an organic acid selected from trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, ethanesulfonic acid, ethanedisulfonic acid, benzenesulfonic acid, adipic acid, cinnamic acid, napadisylic acid and para-toluenesulfonic acid.

Therefore, in one embodiment there is provided a compound of Formula (I) or a pharmaceutically acceptable salt thereof, where the pharmaceutically acceptable salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, ethanesulfonic acid, ethanedisulfonic acid, benzenesulfonic acid, adipic acid, cinnamic acid, napadisylic acid or para-toluenesulfonic acid salt. In one embodiment there is provided a compound of Formula (I) or a pharmaceutically acceptable salt thereof, where the pharmaceutically acceptable salt is a methanesulfonic acid salt. In one embodiment there is provided a compound of Formula (I) or a pharmaceutically acceptable salt thereof, where the pharmaceutically acceptable salt is a mono-methanesulfonic acid salt, i.e. the stoichiometry of the compound of the compound of Formula (I) to methanesulfonic acid is 1:1.

A further embodiment provides any of the embodiments defined herein (for example the embodiment of claim 1) with the proviso that one or more specific Examples (for instance one, two or three specific Examples) selected from the group consisting of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, and 56 is individually disclaimed.

Some values of variable groups in Formula (I) are as follows. Such values may be used in combination with any of the definitions, claims (for example claim 1), or embodiments defined herein to provide further embodiments.
a) R² is methyl.
b) R² is hydro.
c) R¹ is methyl and R² is hydro or methyl.
d) R¹ and R² are both methyl.
e) R¹ and R² are both methyl; or R¹ and R² together with the nitrogen atom to which they are bonded form an azetidinyl, pyrrolidinyl or piperidinyl ring.
f) R¹ and R² are both methyl; or R¹ and R² together with the nitrogen atom to which they are bonded form an azetidinyl ring.
g) R¹ and R² are both methyl; or R¹ and R² together with the nitrogen atom to which they are bonded form a pyrrolidinyl ring.
h) R¹ and R² are both methyl; or R¹ and R² together with the nitrogen atom to which they are bonded form a piperidinyl ring.
i) R¹ and R² together with the nitrogen atom to which they are bonded form an azetidinyl, pyrrolidinyl or piperidinyl ring.
j) R¹ and R² together with the nitrogen atom to which they are bonded form an azetidinyl ring.
k) R¹ and R² together with the nitrogen atom to which they are bonded form a pyrrolidinyl ring.
l) R¹ and R² together with the nitrogen atom to which they are bonded form a piperidinyl ring.
m) R³ is isopropyl, cyclobutyl, 3-methoxycyclobut-1-yl, 3-methoxycyclopent-1-yl, 3-methoxycyclohex-1-yl, 4-methoxycyclohex-1-yl, tetrahydrofuran-3-yl, tetrahydropyran-3-yl or tetrahydropyran-4-yl.
n) R³ is isopropyl, cyclobutyl, cis-3-methoxycyclobut-1-yl, trans-3-methoxycyclobut-1-yl, cis-3-methoxycyclopent-1-yl, trans-3-methoxycyclopent-1-yl, cis-3-methoxycyclohex-1-yl, trans-3-methoxycyclohex-1-yl, cis-4-methoxycyclohex-1-yl, trans-4-methoxycyclohex-1-yl, (3S)-tetrahydrofuran-3-yl, (3S)-tetrahydropyran-3-yl, (3R)-tetrahydropyran-3-yl or tetrahydropyran-4-yl.
o) R³ is isopropyl, cyclobutyl, cis-3-methoxycyclobut-1-yl, trans-3-methoxycyclobut-1-yl, (1S, 3R)-3-methoxycyclopent-1-yl, (1R, 3S)-3-methoxycyclopent-1-yl-3-methoxycyclopent-1-yl, (1S, 3S)-3-methoxycyclopent-1-yl, (1R, 3R)-3-methoxycyclopent-1-yl, (1S, 3R)-3-methoxycyclohex-1-yl, (1R, 3S)-3-methoxycyclohex-1-yl, (1S, 3S)-3-methoxycyclohex-1-yl, (1R, 3R)-3-methoxycyclohex-1-yl, cis-4-methoxycyclohex-1-yl, trans-4-methoxycyclohex-1-yl, (3S)-tetrahydrofuran-3-yl, (3S)-tetrahydropyran-3-yl, (3R)-tetrahydropyran-3-yl or tetrahydropyran-4-yl.
p) R³ is isopropyl.
q) R³ is C₄-C₆ cycloalkyl optionally substituted with one methoxy group.
r) R³ is cyclobutyl, 3-methoxycyclobut-1-yl, 3-methoxycyclopent-1-yl, 3-methoxycyclohex-1-yl or 4-methoxycyclohex-1-yl.
s) R³ is cyclobutyl, cis-3-methoxycyclobut-1-yl, trans-3-methoxycyclobut-1-yl, cis-3-methoxycyclopent-1-yl, trans-3-methoxycyclopent-1-yl, cis-3-methoxycyclohex-1-yl, trans-3-methoxycyclohex-1-yl, cis-4-methoxycyclohex-1-yl or trans-4-methoxycyclohex-1l-yl.
t) R³ is cyclobutyl, cis-3-methoxycyclobut-1-yl, trans-3-methoxycyclobut-1-yl, (1S, 3R)-3-methoxycyclopent-1-yl, (1R, 3S)-3-methoxycyclopent-1-yl-3-methoxycyclopent-1-yl, (1S, 3S)-3-methoxycyclopent-1-yl, (1R, 3R)-3-methoxycyclopent-1-yl, (1S, 3R)-3-methoxycyclohex-1-yl, (1R, 3S)-3-methoxycyclohex-1-yl, (1S, 3S)-3-methoxycyclohex-1-yl, (1R, 3R)-3-methoxycyclohex-1-yl, cis-4-methoxycyclohex-1-yl or trans-4-methoxycyclohex-1-yl.
u) R³ is tetrahydropyranyl or tetrahydrofuranyl.
v) R³ is (3S)-tetrahydrofuran-3-yl, (3S)-tetrahydropyran-3-yl, (3R)-tetrahydropyran-3-yl or tetrahydropyran-4-yl.
w) R⁴ is hydro.
x) R⁴ is methyl.
y) R⁵ is hydro.
z) R⁵ is fluoro.
aa) x is 1.
bb) x is 2.

In one embodiment there is provided a compound of Formula (I), or a pharmaceutically acceptable salt thereof, where:

R¹ and R² are both methyl; or R¹ and R² together with the nitrogen atom to which they are bonded form an azetidinyl, pyrrolidinyl or piperidinyl ring;

x is 1 or 2;

R³ is isopropyl, cyclobutyl, 3-methoxycyclobut-1-yl, 3-methoxycyclopent-1-yl, 3-methoxycyclohex-1-yl, 4-methoxycyclohex-1-yl, tetrahydrofuran-3-yl, tetrahydropyran-3-yl or tetrahydropyran-4-yl;

R⁴ is methyl; and

R⁵ is hydro or fluoro.

In one embodiment there is provided a compound of Formula (I), or a pharmaceutically acceptable salt thereof, where:

R¹ and R² are both methyl;

x is 1 or 2;

R³ is isopropyl, cyclobutyl, 3-methoxycyclobut-1-yl, 3-methoxycyclopent-1-yl, 3-methoxycyclohex-1-yl, 4-methoxycyclohex-1-yl, tetrahydrofuran-3-yl, tetrahydropyran-3-yl or tetrahydropyran-4-yl;

R⁴ is methyl; and

R⁵ is hydro or fluoro.

In one embodiment there is provided a compound of Formula (I), or a pharmaceutically acceptable salt thereof, where:

R¹ and R² together with the nitrogen atom to which they are bonded form an azetidinyl, pyrrolidinyl or piperidinyl ring;

x is 1 or 2;

R³ is isopropyl, cyclobutyl, 3-methoxycyclobut-1-yl, 3-methoxycyclopent-1-yl, 3-methoxycyclohex-1-yl, 4-methoxycyclohex-1-yl, tetrahydrofuran-3-yl, tetrahydropyran-3-yl or tetrahydropyran-4-yl;

R⁴ is methyl; and

R⁵ is hydro or fluoro.

In one embodiment there is provided a compound of Formula (I), or a pharmaceutically acceptable salt thereof, where:

R¹ and R² are both methyl; or R¹ and R² together with the nitrogen atom to which they are bonded form an azetidinyl, pyrrolidinyl or piperidinyl ring;

x is 1 or 2;

R³ is isopropyl;

R⁴ is methyl; and

R⁵ is hydro or fluoro.

In one embodiment there is provided a compound of Formula (I), or a pharmaceutically acceptable salt thereof, where:

R¹ and R² are both methyl; or R¹ and R² together with the nitrogen atom to which they are bonded form an azetidinyl, pyrrolidinyl or piperidinyl ring;

x is 1 or 2;

R³ is cyclobutyl, 3-methoxycyclobut-1-yl, 3-methoxycyclopent-1-yl, 3-methoxycyclohex-1-yl or 4-methoxycyclohex-1-yl;

R⁴ is methyl; and

R⁵ is hydro or fluoro.

In one embodiment there is provided a compound of Formula (I), or a pharmaceutically acceptable salt thereof, where:

R¹ and R² are both methyl; or R¹ and R² together with the nitrogen atom to which they are bonded form an azetidinyl, pyrrolidinyl or piperidinyl ring;

x is 1 or 2;

R³ is tetrahydropyranyl or tetrahydrofuranyl;

R⁴ is methyl; and

R⁵ is hydro or fluoro.

In one embodiment there is provided a compound of Formula (I), or a pharmaceutically acceptable salt thereof, wherein the compound is selected from the group consisting of:
8-[4-[3-(Dimethylamino)propoxy]phenyl]-1-isopropyl-3-methyl-imidazo[4,5-c]quinolin-2-one;
1-Isopropyl-3-methyl-8-[4-(3-pyrrolidin-1-ylpropoxy)phenyl]imidazo[4,5-c]quinolin-2-one;
8-[4-[3-(Azetidin-1-yl)propoxy]phenyl]-1-isopropyl-3-methyl-imidazo[4,5-c]quinolin-2-one;
8-[4-[3-(Azetidin-1-yl)propoxy]phenyl]-7-fluoro-1-isopropyl-3-methyl-imidazo[4,5-c]quinolin-2-one;
8-[4-[2-(Dimethylamino)ethoxy]phenyl]-1-isopropyl-3-methyl-imidazo[4,5-c]quinolin-2-one;
8-[4-[3-(Dimethylamino)propoxy]phenyl]-1-[(1S,3S)-3-methoxycyclopentyl]-3-methyl-imidazo[4,5-c]quinolin-2-one;
8-[4-[3-(Dimethylamino)propoxy]phenyl]-1-[(1R,3R)-3-methoxycyclopentyl]-3-methyl-imidazo[4,5-c]quinolin-2-one;
8-[4-[3-(Dimethylamino)propoxy]phenyl]-3-methyl-1-[(3R)-tetrahydropyran-3-yl]imidazo[4,5-c]quinolin-2-one;
8-[4-[3-(Dimethylamino)propoxy]phenyl]-3-methyl-1-[(3S)-tetrahydropyran-3-yl]imidazo[4,5-c]quinolin-2-one;
8-[4-[3-(Dimethylamino)propoxy]phenyl]-7-fluoro-3-methyl-1-[(3S)-tetrahydropyran-3-yl]imidazo[4,5-c]quinolin-2-one;
8-[4-[3-(Dimethylamino)propoxy]phenyl]-1-(cis-3-methoxycyclobutyl)-3-methyl-imidazo[4,5-c]quinolin-2-one;
8-[4-[3-(Dimethylamino)propoxy]phenyl]-7-fluoro-1-(cis-3-methoxycyclobutyl)-3-methyl-imidazo[4,5-c]quinolin-2-one;
8-[4-[3-(Dimethylamino)propoxy]phenyl]-7-fluoro-1-[trans-3-methoxycyclopentyl]-3-methyl-imidazo[4,5-c]quinolin-2-one;
3-Methyl-8-[4-(3-pyrrolidin-1-ylpropoxy)phenyl]-1-[(3S)-tetrahydropyran-3-yl]imidazo[4,5-c]quinolin-2-one;
3-Methyl-8-[4-(3-pyrrolidin-1-ylpropoxy)phenyl]-1-[(3R)-tetrahydropyran-3-yl]imidazo[4,5-c]quinolin-2-one;
3-Methyl-8-[4-[3-(1-piperidyl)propoxy]phenyl]-1-[(3S)-tetrahydrofuran-3-yl]imidazo[4,5-c]quinolin-2-one;
3-Methyl-8-[4-[3-(1-piperidyl)propoxy]phenyl]-1-tetrahydropyran-4-yl-imidazo[4,5-c]quinolin-2-one;
1-[trans-3-Methoxycyclopentyl]-3-methyl-8-[4-(3-pyrrolidin-1-ylpropoxy)phenyl]imidazo[4,5-c]quinolin-2-one;
8-[4-[3-(Dimethylamino)propoxy]phenyl]-1-(trans-3-methoxycyclobutyl)-3-methyl-imidazo[4,5-c]quinolin-2-one;
1-(trans-4-Methoxycyclohexyl)-3-methyl-8-[4-(3-pyrrolidin-1-ylpropoxy)phenyl]imidazo[4,5-c]quinolin-2-one;
8-[4-[3-(Azetidin-1-yl)propoxy]phenyl]-7-fluoro-1-[trans-3-methoxycyclopentyl]-3-methyl-imidazo[4,5-c]quinolin-2-one;
8-[4-[3-(Dimethylamino)propoxy]phenyl]-1-(cis-4-methoxycyclohexyl)-3-methyl-imidazo[4,5-c]quinolin-2-one;
8-[4-[3-(Dimethylamino)propoxy]phenyl]-1-(cis-4-methoxycyclohexyl)-3-methyl-imidazo[4,5-c]quinolin-2-one;
1-(cis-4-Methoxycyclohexyl)-3-methyl-8-[4-(3-pyrrolidin-1-ylpropoxy)phenyl]imidazo[4,5-c]quinolin-2-one;
8-[4-[3-(Dimethylamino)propoxy]phenyl]-1-[trans-3-methoxycyclohexyl]-3-methyl-imidazo[4,5-c]quinolin-2-one;
8-[4-[3-(Dimethylamino)propoxy]phenyl]-1-[trans-3-methoxycyclohexyl]-3-methyl-imidazo[4,5-c]quinolin-2-one;
1-[trans-3-Methoxycyclohexyl]-3-methyl-8-[4-(3-pyrrolidin-1-ylpropoxy)phenyl]imidazo[4,5-c]quinolin-2-one;
8-[4-[3-(Dimethylamino)propoxy]phenyl]-1-[cis-3-methoxycyclohexyl]-3-methyl-imidazo[4,5-c]quinolin-2-one;
8-[4-[3-(Dimethylamino)propoxy]phenyl]-1-[cis-3-methoxycyclohexyl]-3-methyl-imidazo[4,5-c]quinolin-2-one;
8-[4-[3-(Dimethylamino)propoxy]phenyl]-1-[cis-3-methoxycyclopentyl]-3-methyl-imidazo[4,5-c]quinolin-2-one;
8-[4-[3-(Dimethylamino)propoxy]phenyl]-7-fluoro-1-[cis-3-methoxycyclopentyl]-3-methyl-imidazo[4,5-c]quinolin-2-one;
1-[cis-3-Methoxycyclohexyl]-3-methyl-8-[4-(3-pyrrolidin-1-ylpropoxy)phenyl]imidazo[4,5-c]quinolin-2-one;
1-[cis-3-Methoxycyclohexyl]-3-methyl-8-[4-(3-pyrrolidin-1-ylpropoxy)phenyl]imidazo[4,5-c]quinolin-2-one;
1-[(1S,3S)-3-Methoxycyclopentyl]-3-methyl-8-[4-[3-(1-piperidyl)propoxy]phenyl]imidazo[4,5-c]quinolin-2-one;
1-(cis-3-Methoxycyclobutyl)-3-methyl-8-[4-(3-pyrrolidin-1-ylpropoxy)phenyl]imidazo[4,5-c]quinolin-2-one;
1-(trans-3-Methoxycyclobutyl)-3-methyl-8-[4-(3-pyrrolidin-1-ylpropoxy)phenyl]imidazo[4,5-c]quinolin-2-one;
1-(cis-3-Methoxycyclobutyl)-3-methyl-8-[4-[3-(1-piperidyl)propoxy]phenyl]imidazo[4,5-c]quinolin-2-one;
8-[4-[3-(Dimethylamino)propoxy]phenyl]-3-methyl-1-tetrahydropyran-4-yl-imidazo[4,5-c]quinolin-2-one;
8-[4-[3-(Dimethylamino)propoxy]phenyl]-7-fluoro-3-methyl-1-tetrahydropyran-4-yl-imidazo[4,5-c]quinolin-2-one;
7-Fluoro-3-methyl-8-[4-(3-pyrrolidin-1-ylpropoxy)phenyl]-1-tetrahydropyran-4-yl-imidazo[4,5-c]quinolin-2-one;
8-[4-[2-(Dimethylamino)ethoxy]phenyl]-3-methyl-1-[(3S)-tetrahydropyran-3-yl]imidazo[4,5-c]quinolin-2-one;
8-[4-[2-(Dimethylamino)ethoxy]phenyl]-3-methyl-1-[(3R)-tetrahydropyran-3-yl]imidazo[4,5-c]quinolin-2-one;
1-(3-(cis)Methoxycyclobutyl)-3-methyl-8-[4-(2-pyrrolidin-1-ylethoxy)phenyl]imidazo[4,5-c]quinolin-2-one;
3-Methyl-8-[4-(2-pyrrolidin-1-ylethoxy)phenyl]-1-[(3R)-tetrahydropyran-3-yl]imidazo[4,5-c]quinolin-2-one;
3-Methyl-8-[4-(2-pyrrolidin-1-ylethoxy)phenyl]-1-[(3S)-tetrahydropyran-3-yl]imidazo[4,5-c]quinolin-2-one;
8-[4-[2-(Dimethylamino)ethoxy]phenyl]-1-[(1S,3S)-3-methoxycyclopentyl]-3-methyl-imidazo[4,5-c]quinolin-2-one;
1-Cyclobutyl-8-[4-[2-(dimethylamino)ethoxy]phenyl]-3-methyl-imidazo[4,5-c]quinolin-2-one;
8-[4-[2-(Dimethylamino)ethoxy]phenyl]-3-methyl-1-tetrahydropyran-4-yl-imidazo[4,5-c]quinolin-2-one;
8-[4-[2-(Dimethylamino)ethoxy]phenyl]-1-(3-(cis)methoxycyclobutyl)-3-methyl-imidazo[4,5-c]quinolin-2-one; and
8-[4-[2-(Dimethylamino)ethoxy]phenyl]-7-fluoro-3-methyl-1-tetrahydropyran-4-yl-imidazo[4,5-c]quinolin-2-one.

In one embodiment there is provided a compound of Formula (I), or a pharmaceutically acceptable salt thereof, wherein the compound is selected from the group consisting of:
8-[4-[3-(Dimethylamino)propoxy]phenyl]-1-isopropyl-3-methyl-imidazo[4,5-c]quinolin-2-one;
1-Isopropyl-3-methyl-8-[4-(3-pyrrolidin-1-ylpropoxy)phenyl]imidazo[4,5-c]quinolin-2-one;
8-[4-[3-(Azetidin-1-yl)propoxy]phenyl]-1-isopropyl-3-methyl-imidazo[4,5-c]quinolin-2-one;
8-[4-[3-(Azetidin-1-yl)propoxy]phenyl]-7-fluoro-1-isopropyl-3-methyl-imidazo[4,5-c]quinolin-2-one;
8-[4-[2-(Dimethylamino)ethoxy]phenyl]-1-isopropyl-3-methyl-imidazo[4,5-c]quinolin-2-one;
8-[4-[3-(Dimethylamino)propoxy]phenyl]-1-[(1S,3S)-3-methoxycyclopentyl]-3-methyl-imidazo[4,5-c]quinolin-2-one;
8-[4-[3-(Dimethylamino)propoxy]phenyl]-1-[(1R,3R)-3-methoxycyclopentyl]-3-methyl-imidazo[4,5-c]quinolin-2-one;
8-[4-[3-(Dimethylamino)propoxy]phenyl]-3-methyl-1-[(3R)-tetrahydropyran-3-yl]imidazo[4,5-c]quinolin-2-one;
8-[4-[3-(Dimethylamino)propoxy]phenyl]-3-methyl-1-[(3S)-tetrahydropyran-3-yl]imidazo[4,5-c]quinolin-2-one;
8-[4-[3-(Dimethylamino)propoxy]phenyl]-7-fluoro-3-methyl-1-[(3S)-tetrahydropyran-3-yl]imidazo[4,5-c]quinolin-2-one;
8-[4-[3-(Dimethylamino)propoxy]phenyl]-1-(cis-3-methoxycyclobutyl)-3-methyl-imidazo[4,5-c]quinolin-2-one;
8-[4-[3-(Dimethylamino)propoxy]phenyl]-7-fluoro-1-(cis-3-methoxycyclobutyl)-3-methyl-imidazo[4,5-c]quinolin-2-one;
8-[4-[3-(Dimethylamino)propoxy]phenyl]-7-fluoro-1-[(1S, 3R)-3-methoxycyclopentyl]-3-methyl-imidazo[4,5-c]quinolin-2-one;
8-[4-[3-(Dimethylamino)propoxy]phenyl]-7-fluoro-1-[(1R, 3S)-3-methoxycyclopentyl]-3-methyl-imidazo[4,5-c]quinolin-2-one;
3-Methyl-8-[4-(3-pyrrolidin-1-ylpropoxy)phenyl]-1-[(3S)-tetrahydropyran-3-yl]imidazo[4,5-c]quinolin-2-one;
3-Methyl-8-[4-(3-pyrrolidin-1-ylpropoxy)phenyl]-1-[(3R)-tetrahydropyran-3-yl]imidazo[4,5-c]quinolin-2-one;
3-Methyl-8-[4-[3-(1-piperidyl)propoxy]phenyl]-1-[(3S)-tetrahydrofuran-3-yl]imidazo[4,5-c]quinolin-2-one;
3-Methyl-8-[4-[3-(1-piperidyl)propoxy]phenyl]-1-tetrahydropyran-4-yl-imidazo[4,5-c]quinolin-2-one;
1-[(1S,3S)-3-Methoxycyclopentyl]-3-methyl-8-[4-(3-pyrrolidin-1-ylpropoxy)phenyl]imidazo[4,5-c]quinolin-2-one;
1-[(1R,3R)-3-Methoxycyclopentyl]-3-methyl-8-[4-(3-pyrrolidin-1-ylpropoxy)phenyl]imidazo[4,5-c]quinolin-2-one;
8-[4-[3-(Dimethylamino)propoxy]phenyl]-1-(trans-3-methoxycyclobutyl)-3-methyl-imidazo[4,5-c]quinolin-2-one;
1-((1S,3S)-4-Methoxycyclohexyl)-3-methyl-8-[4-(3-pyrrolidin-1-ylpropoxy)phenyl]imidazo[4,5-c]quinolin-2-one;
1-((1R,3R)-4-Methoxycyclohexyl)-3-methyl-8-[4-(3-pyrrolidin-1-ylpropoxy)phenyl]imidazo[4,5-c]quinolin-2-one;
8-[4-[3-(Azetidin-1-yl)propoxy]phenyl]-7-fluoro-1-[(1S, 3S)-3-methoxycyclopentyl]-3-methyl-imidazo[4,5-c]quinolin-2-one;
8-[4-[3-(Azetidin-1-yl)propoxy]phenyl]-7-fluoro-1-[(1R, 3R)-3-methoxycyclopentyl]-3-methyl-imidazo[4,5-c]quinolin-2-one;
8-[4-[3-(Dimethylamino)propoxy]phenyl]-1-(cis)-4-methoxycyclohexyl)-3-methyl-imidazo[4,5-c]quinolin-2-one;
8-[4-[3-(Dimethylamino)propoxy]phenyl]-1-(trans)-4-methoxycyclohexyl)-3-methyl-imidazo[4,5-c]quinolin-2-one;
1-(cis)-4-Methoxycyclohexyl)-3-methyl-8-[4-(3-pyrrolidin-1-ylpropoxy)phenyl]imidazo[4,5-c]quinolin-2-one;
1-(trans)-4-Methoxycyclohexyl)-3-methyl-8-[4-(3-pyrrolidin-1-ylpropoxy)phenyl]imidazo[4,5-c]quinolin-2-one;
8-[4-[3-(Dimethylamino)propoxy]phenyl]-1-[(1S, 3S)-3-methoxycyclohexyl]-3-methyl-imidazo[4,5-c]quinolin-2-one;
8-[4-[3-(Dimethylamino)propoxy]phenyl]-1-[(1R, 3R)-3-methoxycyclohexyl]-3-methyl-imidazo[4,5-c]quinolin-2-one;
8-[4-[3-(Dimethylamino)propoxy]phenyl]-1-[(1S, 3S)-3-methoxycyclohexyl]-3-methyl-imidazo[4,5-c]quinolin-2-one;
8-[4-[3-(Dimethylamino)propoxy]phenyl]-1-[(1R, 3R)-3-methoxycyclohexyl]-3-methyl-imidazo[4,5-c]quinolin-2-one;
1-[(1S, 3S)-3-Methoxycyclohexyl]-3-methyl-8-[4-(3-pyrrolidin-1-ylpropoxy)phenyl]imidazo[4,5-c]quinolin-2-one;
1-[(1R, 3R)-3-Methoxycyclohexyl]-3-methyl-8-[4-(3-pyrrolidin-1-ylpropoxy)phenyl]imidazo[4,5-c]quinolin-2-one;
8-[4-[3-(Dimethylamino)propoxy]phenyl]-1-[(1S, 3R)-3-methoxycyclohexyl]-3-methyl-imidazo[4,5-c]quinolin-2-one;
8-[4-[3-(Dimethylamino)propoxy]phenyl]-1-[(1R, 3S)-3-methoxycyclohexyl]-3-methyl-imidazo[4,5-c]quinolin-2-one;
8-[4-[3-(Dimethylamino)propoxy]phenyl]-1-[(1S, 3R)-3-methoxycyclohexyl]-3-methyl-imidazo[4,5-c]quinolin-2-one;
8-[4-[3-(Dimethylamino)propoxy]phenyl]-1-[(1R, 3S)-3-methoxycyclohexyl]-3-methyl-imidazo[4,5-c]quinolin-2-one;
8-[4-[3-(Dimethylamino)propoxy]phenyl]-1-[(1S, 3R)-3-methoxycyclopentyl]-3-methyl-imidazo[4,5-c]quinolin-2-one;
8-[4-[3-(Dimethylamino)propoxy]phenyl]-1-[(1R, 3S)-3-methoxycyclopentyl]-3-methyl-imidazo[4,5-c]quinolin-2-one;
8-[4-[3-(Dimethylamino)propoxy]phenyl]-7-fluoro-1-[(1S, 3R)-3-methoxycyclopentyl]-3-methyl-imidazo[4,5-c]quinolin-2-one;
8-[4-[3-(Dimethylamino)propoxy]phenyl]-7-fluoro-1-[(1R, 3S)-3-methoxycyclopentyl]-3-methyl-imidazo[4,5-c]quinolin-2-one;
1-[(1S, 3R)-3-Methoxycyclohexyl]-3-methyl-8-[4-(3-pyrrolidin-1-ylpropoxy)phenyl]imidazo[4,5-c]quinolin-2-one;
1-[(1R, 3S)-3-Methoxycyclohexyl]-3-methyl-8-[4-(3-pyrrolidin-1-ylpropoxy)phenyl]imidazo[4,5-c]quinolin-2-one;
1-[(1S, 3R)-3-Methoxycyclohexyl]-3-methyl-8-[4-(3-pyrrolidin-1-ylpropoxy)phenyl]imidazo[4,5-c]quinolin-2-one;
1-[(1R, 3S)-3-Methoxycyclohexyl]-3-methyl-8-[4-(3-pyrrolidin-1-ylpropoxy)phenyl]imidazo[4,5-c]quinolin-2-one;
1-[(1S,3S)-3-Methoxycyclopentyl]-3-methyl-8-[4-[3-(1-piperidyl)propoxy]phenyl]imidazo[4,5-c]quinolin-2-one;
1-(cis-3-Methoxycyclobutyl)-3-methyl-8-[4-(3-pyrrolidin-1-ylpropoxy)phenyl]imidazo[4,5-c]quinolin-2-one;
1-(trans-3-Methoxycyclobutyl)-3-methyl-8-[4-(3-pyrrolidin-1-ylpropoxy)phenyl]imidazo[4,5-c]quinolin-2-one;
1-(cis-3-Methoxycyclobutyl)-3-methyl-8-[4-[3-(1-piperidyl)propoxy]phenyl]imidazo[4,5-c]quinolin-2-one;
8-[4-[3-(Dimethylamino)propoxy]phenyl]-3-methyl-1-tetrahydropyran-4-yl-imidazo[4,5-c]quinolin-2-one;
8-[4-[3-(Dimethylamino)propoxy]phenyl]-7-fluoro-3-methyl-1-tetrahydropyran-4-yl-imidazo[4,5-c]quinolin-2-one;
7-Fluoro-3-methyl-8-[4-(3-pyrrolidin-1-ylpropoxy)phenyl]-1-tetrahydropyran-4-yl-imidazo[4,5-c]quinolin-2-one;
8-[4-[2-(Dimethylamino)ethoxy]phenyl]-3-methyl-1-[(3S)-tetrahydropyran-3-yl]imidazo[4,5-c]quinolin-2-one;
8-[4-[2-(Dimethylamino)ethoxy]phenyl]-3-methyl-1-[(3R)-tetrahydropyran-3-yl]imidazo[4,5-c]quinolin-2-one;
1-(3-(cis)-Methoxycyclobutyl)-3-methyl-8-[4-(2-pyrrolidin-1-ylethoxy)phenyl]imidazo[4,5-c]quinolin-2-one;
3-Methyl-8-[4-(2-pyrrolidin-1-ylethoxy)phenyl]-1-[(3R)-tetrahydropyran-3-yl]imidazo[4,5-c]quinolin-2-one;
3-Methyl-8-[4-(2-pyrrolidin-1-ylethoxy)phenyl]-1-[(3S)-tetrahydropyran-3-yl]imidazo[4,5-c]quinolin-2-one;
8-[4-[2-(Dimethylamino)ethoxy]phenyl]-1-[(1S,3S)-3-methoxycyclopentyl]-3-methyl-imidazo[4,5-c]quinolin-2-one;
1-Cyclobutyl-8-[4-[2-(dimethylamino)ethoxy]phenyl]-3-methyl-imidazo[4,5-c]quinolin-2-one;
8-[4-[2-(Dimethylamino)ethoxy]phenyl]-3-methyl-1-tetrahydropyran-4-yl-imidazo[4,5-c]quinolin-2-one;
8-[4-[2-(Dimethylamino)ethoxy]phenyl]-1-(3-(cis)methoxycyclobutyl)-3-methyl-imidazo[4,5-c]quinolin-2-one; and
8-[4-[2-(Dimethylamino)ethoxy]phenyl]-7-fluoro-3-methyl-1-tetrahydropyran-4-yl-imidazo[4,5-c]quinolin-2-one.

In one embodiment there is provided 8-[4-[2-(dimethylamino)ethoxy]phenyl]-3-methyl-1-[(3S)-tetrahydropyran-3-yl]imidazo[4,5-c]quinolin-2-one, or a pharmaceutically acceptable salt thereof.

In one embodiment there is provided 8-[4-[2-(dimethylamino)ethoxy]phenyl]-3-methyl-1-[(3S)-tetrahydropyran-3-yl]imidazo[4,5-c]quinolin-2-one.

In one embodiment there is provided a pharmaceutically acceptable salt of 8-[4-[2-(dimethylamino)ethoxy]phenyl]-3-methyl-1-[(3S)-tetrahydropyran-3-yl]imidazo[4,5-c]quinolin-2-one.

In one embodiment there is provided 8-[4-[3-(dimethylamino)propoxy]phenyl]-1-[(1S,3S)-3-methoxycyclopentyl]-3-methyl-imidazo[4,5-c]quinolin-2-one, or a pharmaceutically acceptable salt thereof.

In one embodiment there is provided 8-[4-[3-(dimethylamino)propoxy]phenyl]-1-[(1S,3S)-3-methoxycyclopentyl]-3-methyl-imidazo[4,5-c]quinolin-2-one.

In one embodiment there is provided a pharmaceutically acceptable salt of 8-[4-[3-(dimethylamino)propoxy]phenyl]-1-[(1S,3S)-3-methoxycyclopentyl]-3-methyl-imidazo[4,5-c]quinolin-2-one.

In one embodiment there is provided 8-[4-[3-(dimethylamino)propoxy]phenyl]-1-[(1R,3R)-3-methoxycyclopentyl]-3-methyl-imidazo[4,5-c]quinolin-2-one, or a pharmaceutically acceptable salt thereof.

In one embodiment there is provided 8-[4-[3-(dimethylamino)propoxy]phenyl]-1-[(1R,3R)-3-methoxycyclopentyl]-3-methyl-imidazo[4,5-c]quinolin-2-one.

In one embodiment there is provided a pharmaceutically acceptable salt of 8-[4-[3-(dimethylamino)propoxy]phenyl]-1-[(1R,3R)-3-methoxycyclopentyl]-3-methyl-imidazo[4,5-c]quinolin-2-one.

Compounds and salts described in this specification may exist in solvated forms and unsolvated forms. For example, a solvated form may be a hydrated form, such as a hemi-hydrate, a mono-hydrate, a di-hydrate, a tri-hydrate or an alternative quantity thereof. The invention encompasses all such solvated and unsolvated forms of compounds of Formula (I), particularly to the extent that such forms possess ATM kinase inhibitory activity, as for example measured using the tests described herein.

Atoms of the compounds and salts described in this specification may exist as their isotopes. The invention encompasses all compounds of Formula (I) where an atom is replaced by one or more of its isotopes (for example a compound of Formula (I) where one or more carbon atom is an ¹¹C or ¹³C carbon isotope, or where one or more hydrogen atoms is a ²H or ³H isotope).

Compounds and salts described in this specification may exist as a mixture of tautomers. “Tautomers” are structural isomers that exist in equilibrium resulting from the migration of a hydrogen atom. The invention includes all tautomers of compounds of Formula (I) particularly to the extent that such tautomers possess ATM kinase inhibitory activity.

Compounds of Formula (I) may for example be prepared by the reaction of a compound of Formula (II):

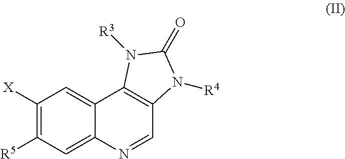

Or a salt thereof, where R³, R⁴ and R⁵ are as defined in any of the embodiments herein and X is a leaving group (for example a halogen atom, or alternatively a fluorine atom) with a compound of formula (III):

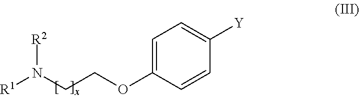

or a salt thereof, where x, R¹ and R² are as defined in any of the embodiments herein and Y is a boronic acid, boronic ester or potassium trifluoroborate group (for example boronic acid, boronic acid pinacol ester, or potassium trifluoroborate). The reaction may be performed under standard conditions well known to those skilled in the art, for example in the presence of a palladium source (for example tetrakis triphenylphosphine palladium or palladium(II) acetate), optionally a phosphine ligand (for example Xantphos or S-phos), and a suitable base (for example cesium carbonate or triethylamine).

Compounds of Formula (II) are therefore useful as intermediates in the preparation of the compounds of Formula (I) and provide a further embodiment.

In one embodiment there is provided a compound of Formula (II), or a salt thereof, where:

R³ is isopropyl, C₄-C₆cycloalkyl optionally substituted with one methoxy group, tetrahydrofuranyl or tetrahydropyranyl;

R⁴ is hydro or methyl;

R⁵ is hydro or fluoro; and

X is a leaving group. In one embodiment X is an iodine, bromine, or chlorine atom or a triflate group. In one embodiment X is a bromine atom.

In one embodiment there is provided a compound of Formula (II), or a salt thereof, where:

R³ is isopropyl, cyclobutyl, 3-methoxycyclobut-1-yl, 3-methoxycyclopent-1-yl, 3-methoxycyclohex-1-yl, 4-methoxycyclohex-1-yl, tetrahydrofuran-3-yl, tetrahydropyran-3-yl or tetrahydropyran-4-yl;

R⁴ is methyl;

R⁵ is hydro or fluoro; and

X is a leaving group. In one embodiment X is an iodine, bromine, or chlorine atom or a triflate group. In one embodiment X is a bromine atom.

In any of the embodiments where a compound of Formula (II) or a salt thereof is mentioned it is to be understood that such salts do not need to be pharmaceutically acceptable salts. A suitable salt of a compound of Formula (II) is, for example, an acid-addition salt. An acid addition salt of a compound of Formula (II) may be formed by bringing the compound into contact with a suitable inorganic or organic acid under conditions known to the skilled person. An acid addition salt may for example be formed using an inorganic acid selected from hydrochloric acid, hydrobromic acid, sulphuric acid and phosphoric acid. An acid addition salt may also be formed using an organic acid selected from trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, ethanesulfonic acid, ethanedisulfonic acid, benzenesulfonic acid, adipic acid, cinnamic acid, napadisylic acid and para-toluenesulfonic acid.

Therefore, in one embodiment there is provided a compound of Formula (II) or a salt thereof, where the salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, ethanesulfonic acid, ethanedisulfonic acid, benzenesulfonic acid, adipic acid, cinnamic acid, napadisylic acid or para-toluenesulfonic acid salt.

In one embodiment there is provided a compound of Formula (II), or a salt thereof, wherein the compound is selected from the group consisting of:
8-Bromo-7-fluoro-1-isopropyl-3-methyl-imidazo[4,5-c]quinolin-2-one;
8-Bromo-1-isopropyl-3-methyl-imidazo[4,5-c]quinolin-2-one;
8-Bromo-1-[(1S,3S)-3-methoxycyclopentyl]-3-methyl-imidazo[4,5-c]quinolin-2-one;
8-Bromo-3-methyl-1-(oxan-4-yl)imidazo[5,4-c]quinolin-2-one;
8-Bromo-1-(cis-3-methoxycyclobutyl)-3-methylimidazo[4,5-c]quinolin-2-one;
8-Bromo-7-fluoro-1-(cis-3-methoxycyclobutyl)-3-methylimidazo[4,5-c]quinolin-2-one;
8-bromo-3-methyl-1-[(3S)-oxan-3-yl]imidazo[5,4-c]quinolin-2-one;
8-bromo-3-methyl-1-[(3R)-oxan-3-yl]imidazo[5,4-c]quinolin-2-one;
8-bromo-7-fluoro-3-methyl-1-(oxan-4-yl)imidazo[5,4-c]quinolin-2-one;
8-bromo-7-fluoro-3-methyl-1-[(3S)-oxan-3-yl]imidazo[5,4-c]quinolin-2-one;
8-bromo-7-fluoro-3-methyl-1-[(3R)-oxan-3-yl]imidazo[5,4-c]quinolin-2-one;
8-bromo-3-methyl-1-[(3S)-tetrahydrofuran-3-yl]imidazo[4,5-c]quinolin-2-one;
8-bromo-1-cyclobutyl-3-methyl-imidazo[4,5-c]quinolin-2-one;
8-Bromo-1-(trans-3-methoxycyclobutyl)-3-methyl-imidazo[4,5-c]quinolin-2-one;
8-bromo-1-(trans-4-methoxycyclohexyl)-3-methyl-imidazo[4,5-c]quinolin-2-one;
8-bromo-1-(cis-4-methoxycyclohexyl)-3-methyl-imidazo[4,5-c]quinolin-2-one;
8-bromo-1-[(3-methoxycyclohexyl]-3-methyl-imidazo[4,5-c]quinolin-2-one;
8-bromo-1-[(trans-3-methoxycyclohexyl]-3-methyl-imidazo[4,5-c]quinolin-2-one;
8-bromo-1-[(cis-3-methoxycyclohexyl]-3-methyl-imidazo[4,5-c]quinolin-2-one;
and
8-bromo-1-[(cis-3-methoxycyclopentyl]-3-methyl-imidazo[4,5-c]quinolin-2-one.

Compounds of formula (III) and (IV) can be prepared by methods similar to those shown in the Examples section.

In one embodiment there is provided any one of the novel intermediates described in the experimental section.

As a result of their ATM kinase inhibitory activity, the compounds of Formula (I), and pharmaceutically acceptable salts thereof are expected to be useful in therapy, for example in the treatment of diseases or medical conditions mediated at least in part by ATM kinase, including cancer.

Where “cancer” is mentioned, this includes both non-metastatic cancer and also metastatic cancer, such that treating cancer involves treatment of both primary tumours and also tumour metastases.

“ATM kinase inhibitory activity” refers to a decrease in the activity of ATM kinase as a direct or indirect response to the presence of a compound of Formula (I), or to pharmaceutically acceptable salt thereof, relative to the activity of ATM kinase in the absence of compound of Formula (I), or pharmaceutically acceptable salt thereof. Such a decrease in activity may be due to the direct interaction of the compound of Formula (I), or pharmaceutically acceptable salt thereof with ATM kinase, or due to the interaction of the compound of Formula (I), or pharmaceutically acceptable salt thereof with one or more other factors that in turn affect ATM kinase activity. For example, the compound of Formula (I), or pharmaceutically acceptable salt thereof may decrease ATM kinase by directly binding to the ATM kinase, by causing (directly or indirectly) another factor to decrease ATM kinase activity, or by (directly or indirectly) decreasing the amount of ATM kinase present in the cell or organism.

The term “therapy” is intended to have its normal meaning of dealing with a disease in order to entirely or partially relieve one, some or all of its symptoms, or to correct or compensate for the underlying pathology. The term “therapy” also includes “prophylaxis” unless there are specific indications to the contrary. The terms “therapeutic” and “therapeutically” should be interpreted in a corresponding manner.

The term “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly the term “treat” can be regarded as “applying therapy” where “therapy” is as defined herein.

In one embodiment there is provided a compound of Formula (I), or a pharmaceutically acceptable salt thereof, for use in therapy.

In one embodiment there is provided the use of the compound of Formula (I), or a pharmaceutically acceptable salt thereof, in the manufacture of a medicament.

In one embodiment there is provided a compound of Formula (I), or a pharmaceutically acceptable salt thereof, for use in the treatment of a disease mediated by ATM kinase.

In one embodiment there is provided a compound of Formula (I), or a pharmaceutically acceptable salt thereof, for use in the treatment of a disease mediated by ATM kinase, where the disease mediated by ATM kinase is cancer.

In one embodiment there is provided a compound of Formula (I), or a pharmaceutically acceptable salt thereof, for use in the treatment of a disease mediated by ATM kinase, where the disease mediated by ATM kinase is colorectal cancer, glioblastoma, gastric cancer, ovarian cancer, diffuse large B-cell lymphoma, chronic lymphocytic leukaemia, acute myeloid leukaemia, head and neck squamous cell carcinoma, breast cancer, hepatocellular carcinoma, small cell lung cancer or non-small cell lung cancer.

In one embodiment there is provided a compound of Formula (I), or a pharmaceutically acceptable salt thereof, for use in the treatment of a disease mediated by ATM kinase, where the disease mediated by ATM kinase is colorectal cancer.

In one embodiment there is provided a compound of Formula (I), or a pharmaceutically acceptable salt thereof, for use in the treatment of cancer.

In one embodiment there is provided a compound of Formula (I), or a pharmaceutically acceptable salt thereof, for use in the treatment of colorectal cancer, glioblastoma, gastric cancer, ovarian cancer, diffuse large B-cell lymphoma, chronic lymphocytic leukaemia, acute myeloid leukaemia, head and neck squamous cell carcinoma, breast cancer, hepatocellular carcinoma, small cell lung cancer or non-small cell lung cancer.

In one embodiment there is provided a compound of Formula (I), or a pharmaceutically acceptable salt thereof, for use in the treatment of colorectal cancer.

In one embodiment there is provided a compound of Formula (I), or a pharmaceutically acceptable salt thereof, for use in the treatment of Huntingdon's disease.

In one embodiment there is provided a compound of Formula (I), or a pharmaceutically acceptable salt thereof, for use as a neuroprotective agent.

A “neuroprotective agent” is an agent that preserves neuronal structure and/or function.

In one embodiment there is provided the use of the compound of Formula (I), or a pharmaceutically acceptable salt thereof, in the manufacture of a medicament for the treatment of a disease mediated by ATM kinase.

In one embodiment there is provided the use of the compound of Formula (I), or a pharmaceutically acceptable salt thereof, in the manufacture of a medicament for the treatment of a disease mediated by ATM kinase, where the disease mediated by ATM kinase is cancer.

In one embodiment there is provided the use of the compound of Formula (I), or a pharmaceutically acceptable salt thereof, in the manufacture of a medicament for the treatment of a disease mediated by ATM kinase, where the disease mediated by ATM kinase is colorectal cancer, glioblastoma, gastric cancer, ovarian cancer, diffuse large B-cell lymphoma, chronic lymphocytic leukaemia, acute myeloid leukaemia, head and neck squamous cell carcinoma, breast cancer, hepatocellular carcinoma, small cell lung cancer and non-small cell lung cancer.

In one embodiment there is provided the use of the compound of Formula (I), or a pharmaceutically acceptable salt thereof, in the manufacture of a medicament for the treatment of a disease mediated by ATM kinase, where the disease mediated by ATM kinase is colorectal cancer.

In one embodiment there is provided the use of the compound of Formula (I), or a pharmaceutically acceptable salt thereof, in the manufacture of a medicament for the treatment of cancer.

In one embodiment there is provided the use of the compound of Formula (I), or a pharmaceutically acceptable salt thereof, in the manufacture of a medicament for the treatment of colorectal cancer, glioblastoma, gastric cancer, ovarian cancer, diffuse large B-cell lymphoma, chronic lymphocytic leukaemia, acute myeloid leukaemia, head and neck squamous cell carcinoma, breast cancer, hepatocellular carcinoma, small cell lung cancer or non-small cell lung cancer.

In one embodiment there is provided the use of the compound of Formula (I), or a pharmaceutically acceptable salt thereof, in the manufacture of a medicament for the treatment of colorectal cancer.

In one embodiment there is provided the use of the compound of Formula (I), or a pharmaceutically acceptable salt thereof, in the manufacture of a medicament for the treatment of Huntingdon's disease.

In one embodiment there is provided the use of the compound of Formula (I), or a pharmaceutically acceptable salt thereof, in the manufacture of a medicament for use as a neuroprotective agent.

In one embodiment there is provided a method for treating a disease in which inhibition of ATM kinase is beneficial in a warm-blooded animal in need of such treatment, which comprises administering to said warm-blooded animal a therapeutically effective amount of a compound of Formula (I), or a pharmaceutically acceptable salt thereof.

The term “therapeutically effective amount” refers to an amount of a compound of Formula (I) as described in any of the embodiments herein which is effective to provide “therapy” in a subject, or to “treat” a disease or disorder in a subject. In the case of cancer, the therapeutically effective amount may cause any of the changes observable or measurable in a subject as described in the definition of“therapy”, “treatment” and “prophylaxis” above. For example, the effective amount can reduce the number of cancer or tumour cells; reduce the overall tumour size; inhibit or stop tumour cell infiltration into peripheral organs including, for example, the soft tissue and bone; inhibit and stop tumour metastasis; inhibit and stop tumour growth; relieve to some extent one or more of the symptoms associated with the cancer; reduce morbidity and mortality; improve quality of life; or a combination of such effects. An effective amount may be an amount sufficient to decrease the symptoms of a disease responsive to inhibition of ATM kinase activity. For cancer therapy, efficacy in-vivo can, for example, be measured by assessing the duration of survival, time to disease progression (TTP), the response rates (RR), duration of response, and/or quality of life. As recognized by those skilled in the art, effective amounts may vary depending on route of administration, excipient usage, and co-usage with other agents. For example, where a combination therapy is used, the amount of the compound of formula (I) or pharmaceutcially acceptable salt described in this specification and the amount of the other pharmaceutically active agent(s) are, when combined, jointly effective to treat a targeted disorder in the animal patient. In this context, the combined amounts are in a “therapeutically effective amount” if they are, when combined, sufficient to decrease the symptoms of a disease responsive to inhibition of ATM activity as described above. Typically, such amounts may be determined by one skilled in the art by, for example, starting with the dosage range described in this specification for the compound of formula (I) or pharmaceutcially acceptable salt thereof and an approved or otherwise published dosage range(s) of the other pharmaceutically active compound(s).

“Warm-blooded animals” include, for example, humans.

In one embodiment there is provided a method for treating a disease in which inhibition of ATM kinase is beneficial in a warm-blooded animal in need of such treatment, which comprises administering to said warm-blooded animal a therapeutically effective amount of a compound of Formula (I), or a pharmaceutically acceptable salt thereof, and where the disease in which inhibition of ATM kinase is beneficial is cancer.

In one embodiment there is provided a method for treating a disease in which inhibition of ATM kinase is beneficial in a warm-blooded animal in need of such treatment, which comprises administering to said warm-blooded animal a therapeutically effective amount of a compound of Formula (I), or a pharmaceutically acceptable salt thereof, and where the disease in which inhibition of ATM kinase is beneficial is colorectal cancer, glioblastoma, gastric cancer, ovarian cancer, diffuse large B-cell lymphoma, chronic lymphocytic leukaemia, acute myeloid leukaemia, head and neck squamous cell carcinoma, breast cancer, hepatocellular carcinoma, small cell lung cancer or non-small cell lung cancer.

In one embodiment there is provided a method for treating a disease in which inhibition of ATM kinase is beneficial in a warm-blooded animal in need of such treatment, which comprises administering to said warm-blooded animal a therapeutically effective amount of a compound of Formula (I), or a pharmaceutically acceptable salt thereof, and where the disease in which inhibition of ATM kinase is beneficial is colorectal cancer.

In one embodiment there is provided a method for treating a disease in which inhibition of ATM kinase is beneficial in a warm-blooded animal in need of such treatment, which comprises administering to said warm-blooded animal a therapeutically effective amount of a compound of Formula (I), or a pharmaceutically acceptable salt thereof, and where the disease in which inhibition of ATM kinase is beneficial is Huntingdon's disease.

In one embodiment there is provided a method for treating cancer in a warm-blooded animal in need of such treatment, which comprises administering to said warm-blooded animal a therapeutically effective amount of a compound of Formula (I), or a pharmaceutically acceptable salt thereof.

In one embodiment there is provided a method for treating colorectal cancer, glioblastoma, gastric cancer, ovarian cancer, diffuse large B-cell lymphoma, chronic lymphocytic leukaemia, acute myeloid leukaemia, head and neck squamous cell carcinoma, breast cancer, hepatocellular carcinoma, small cell lung cancer or non-small cell lung cancer in a warm-blooded animal in need of such treatment, which comprises administering to said warm-blooded animal a therapeutically effective amount of a compound of Formula (I), or a pharmaceutically acceptable salt thereof.

In one embodiment there is provided a method for treating colorectal cancer in a warm-blooded animal in need of such treatment, which comprises administering to said warm-blooded animal a therapeutically effective amount of a compound of Formula (I), or a pharmaceutically acceptable salt thereof.

In one embodiment there is provided a method for treating Huntingdon's disease in a warm-blooded animal in need of such treatment, which comprises administering to said warm-blooded animal a therapeutically effective amount of a compound of Formula (I), or a pharmaceutically acceptable salt thereof.

In one embodiment there is provided a method for effecting neuroprotection in a warm-blooded animal in need of such treatment, which comprises administering to said warm-blooded animal a therapeutically effective amount of a compound of Formula (I), or a pharmaceutically acceptable salt thereof.

In one embodiment there is provided a method for treating cancer in a warm-blooded animal in need of such treatment, which comprises administering to said warm-blooded animal a therapeutically effective amount of a compound of Formula (I), or a pharmaceutically acceptable salt thereof. In one embodiment, said cancer is selected from colorectal cancer, glioblastoma, gastric cancer, ovarian cancer, diffuse large B-cell lymphoma, chronic lymphocytic leukaemia, acute myeloid leukaemia, head and neck squamous cell carcinoma, breast cancer, hepatocellular carcinoma, small cell lung cancer and non-small cell lung cancer. In one embodiment, said cancer is selected from colorectal cancer, glioblastoma, gastric cancer, ovarian cancer, diffuse large B-cell lymphoma, chronic lymphocytic leukaemia, head and neck squamous cell carcinoma and lung cancer. In one embodiment, said cancer is colorectal cancer.

In any embodiment where cancer is mentioned in a general sense, said cancer may be selected from colorectal cancer, glioblastoma, gastric cancer, ovarian cancer, diffuse large B-cell lymphoma, chronic lymphocytic leukaemia, acute myeloid leukaemia, head and neck squamous cell carcinoma, breast cancer, hepatocellular carcinoma, small cell lung cancer and non-small cell lung cancer.

In any embodiment where cancer is mentioned in a general sense the following embodiments may apply:

In one embodiment the cancer is colorectal cancer.

In one embodiment the cancer is glioblastoma.

In one embodiment the cancer is gastric cancer.

In one embodiment the cancer is oesophageal cancer.

In one embodiment the cancer is ovarian cancer.

In one embodiment the cancer is endometrial cancer.

In one embodiment the cancer is cervical cancer.

In one embodiment the cancer is diffuse large B-cell lymphoma.

In one embodiment the cancer is chronic lymphocytic leukaemia.

In one embodiment the cancer is acute myeloid leukaemia.

In one embodiment the cancer is head and neck squamous cell carcinoma.

In one embodiment the cancer is breast cancer. In one embodiment the cancer is triple negative breast cancer.

“Triple negative breast cancer” is any breast cancer that does not express the genes for the oestrogen receptor, progesterone receptor and Her2/neu.

In one embodiment the cancer is hepatocellular carcinoma.

In one embodiment the cancer is lung cancer. In one embodiment the lung cancer is small cell lung cancer. In one embodiment the lung cancer is non-small cell lung cancer.

In one embodiment the cancer is non-metastatic cancer. In one embodiment the cancer is metastatic cancer. In one embodiment the metastatic cancer comprises metastases of the central nervous system. In one embodiment the metastases of the central nervous system comprise brain metastases. In one embodiment the metastases of the central nervous system comprise leptomeningeal metastases.

“Leptomeningeal metastases” occur when cancer spreads to the meninges, the layers of tissue that cover the brain and the spinal cord. Metastases can spread to the meninges through the blood or they can travel from brain metastases, carried by the cerebrospinal fluid (CSF) that flows through the meninges.

The anti-cancer treatment described in this specification may be useful as a sole therapy, or may involve, in addition to administration of the compound of Formula (I), conventional surgery, radiotherapy or chemotherapy; or a combination of such additional therapies. Such conventional surgery, radiotherapy or chemotherapy may be administered simultaneously, sequentially or separately to treatment with the compound of Formula (I).

Radiotherapy may include one or more of the following categories of therapy:
i. External radiation therapy using electromagnetic radiation, and intraoperative radiation therapy using electromagnetic radiation;
ii. Internal radiation therapy or brachytherapy; including interstitial radiation therapy or intraluminal radiation therapy; or
iii. Systemic radiation therapy, including but not limited to iodine 131 and strontium 89.

In one embodiment there is provided a compound of Formula (I), or a pharmaceutically acceptable salt thereof, for use in the treatment of cancer, where the compound of Formula (I), or a pharmaceutically acceptable salt thereof, is administered in combination with radiotherapy. In one embodiment the radiotherapy is selected from one or more of the categories of radiotherapy listed under points (i)-(iii) above.

In one embodiment there is provided a compound of Formula (I), or a pharmaceutically acceptable salt thereof, for use in the treatment of glioblastoma, lung cancer (for example small cell lung cancer or non-small cell lung cancer), breast cancer (for example triple negative breast cancer), head and neck squamous cell carcinoma, oesophageal cancer, cervical cancer or endometrial cancer, where the compound of Formula (I), or a pharmaceutically acceptable salt thereof, is administered in combination with radiotherapy. In one embodiment the radiotherapy is selected from one or more of the categories of radiotherapy listed under points (i)-(iii) above.

In one embodiment there is provided a compound of Formula (I), or a pharmaceutically acceptable salt thereof, for use in the treatment of glioblastoma, where the compound of Formula (I), or a pharmaceutically acceptable salt thereof, is administered in combination with radiotherapy. In one embodiment the radiotherapy is selected from one or more of the categories of radiotherapy listed under points (i)-(iii) above.

In one embodiment there is provided a compound of Formula (I), or a pharmaceutically acceptable salt thereof, for use in the treatment of metastatic cancer, where the compound of Formula (I), or a pharmaceutically acceptable salt thereof, is administered in combination with radiotherapy. In one embodiment the radiotherapy is selected from one or more of the categories of radiotherapy listed under points (i)-(iii) above.

In one embodiment there is provided a compound of Formula (I), or a pharmaceutically acceptable salt thereof, for use in the treatment of metastases of the central nervous system, where the compound of Formula (I), or a pharmaceutically acceptable salt thereof, is administered in combination with radiotherapy. In one embodiment the radiotherapy is selected from one or more of the categories of radiotherapy listed under points (i)-(iii) above.

In one embodiment there is provided a compound of Formula (I), or a pharmaceutically acceptable salt thereof, for use in the treatment of leptomeningeal metastases, where the compound of Formula (I), or a pharmaceutically acceptable salt thereof, is administered in combination with radiotherapy. In one embodiment the radiotherapy is selected from one or more of the categories of radiotherapy listed under points (i)-(iii) above.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with radiotherapy. In one embodiment the radiotherapy is selected from one or more of the categories of radiotherapy listed under points (i)-(iii) above.

In one embodiment there is provided a method of treating cancer in a warm-blooded animal who is in need of such treatment, which comprises administering to said warm-blooded animal a compound of Formula (I), or a pharmaceutically acceptable salt thereof and radiotherapy, wherein the compound of Formula (I), or a pharmaceutically acceptable salt thereof, and radiotherapy are jointly effective in producing an anti-cancer effect. In one embodiment the cancer is selected from glioblastoma, lung cancer (for example small cell lung cancer or non-small cell lung cancer), breast cancer (for example triple negative breast cancer), head and neck squamous cell carcinoma, oesophageal cancer, cervical cancer and endometrial cancer. In one embodiment the cancer is glioblastoma. In one embodiment, the cancer is metastatic cancer. In one embodiment the metastatic cancer comprises metastases of the central nervous system. In one embodiment the metastases of the central nervous system comprise brain metastases. In one embodiment the metastases of the central nervous system comprise leptomeningeal metastases. In any embodiment the radiotherapy is selected from one or more of the categories of radiotherapy listed under points (i)-(iii) above.

In one embodiment there is provided a method of treating cancer in a warm-blooded animal who is in need of such treatment, which comprises administering to said warm-blooded animal a compound of Formula (I), or a pharmaceutically acceptable salt thereof and simultaneously, separately or sequentially administering radiotherapy, wherein the compound of Formula (I), or a pharmaceutically acceptable salt thereof, and radiotherapy are jointly effective in producing an anti-cancer effect. In one embodiment the cancer is glioblastoma. In one embodiment, the cancer is metastatic cancer. In one embodiment the metastatic cancer comprises metastases of the central nervous system. In one embodiment the metastases of the central nervous system comprise brain metastases.

In one embodiment the metastases of the central nervous system comprise leptomeningeal metastases. In any embodiment the radiotherapy is selected from one or more of the categories of radiotherapy listed under points (i)-(iii) above.

Chemotherapy may include one or more of the following categories of anti-tumour substance:
i. Antineoplastic agents and combinations thereof, such as DNA alkylating agents (for example cisplatin, oxaliplatin, carboplatin, cyclophosphamide, nitrogen mustards like ifosfamide, bendamustine, melphalan, chlorambucil, busulphan, temozolamide and nitrosoureas like carmustine); antimetabolites (for example gemcitabine and antifolates such as fluoropyrimidines like 5-fluorouracil and tegafur, raltitrexed, methotrexate, cytosine arabinoside, and hydroxyurea); anti-tumour antibiotics (for example anthracyclines like adriamycin, bleomycin, doxorubicin, liposomal doxorubicin, pirarubicin, daunomycin, valrubicin, epirubicin, idarubicin, mitomycin-C, dactinomycin, amrubicin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, irinotecan, topotecan and camptothecin); inhibitors of DNA repair mechanisms such as CHK kinase; DNA-dependent protein kinase inhibitors; inhibitors of poly (ADP-ribose) polymerase (PARP inhibitors, including olaparib); and Hsp90 inhibitors such as tanespimycin and retaspimycin, inhibitors of ATR kinase (such as AZD6738); and inhibitors of WEE1 kinase (such as AZD1775/MK-1775);
ii. Antiangiogenic agents such as those that inhibit the effects of vascular endothelial growth factor, for example the anti-vascular endothelial cell growth factor antibody bevacizumab and for example, a VEGF receptor tyrosine kinase inhibitor such as vandetanib (ZD6474), sorafenib, vatalanib (PTK787), sunitinib (SU11248), axitinib (AG-013736), pazopanib (GW 786034) and cediranib (AZD2171); compounds such as those disclosed in International Patent Applications WO97/22596, WO 97/30035, WO 97/32856 and WO 98/13354; and compounds that work by other mechanisms (for example linomide, inhibitors of integrin αvβ3 function and angiostatin), or inhibitors of angiopoietins and their receptors (Tie-1 and Tie-2), inhibitors of PLGF, inhibitors of delta-like ligand (DLL-4);
iii. Immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor; approaches to decrease T-cell anergy or regulatory T-cell function; approaches that enhance T-cell responses to tumours, such as blocking antibodies to CTLA4 (for example ipilimumab and tremelimumab), B7H1, PD-1 (for example BMS-936558 or AMP-514), PD-L1 (for example MEDI4736) and agonist antibodies to CD137; approaches using transfected immune cells such as cytokine-transfected dendritic cells; approaches using cytokine-transfected tumour cell lines, approaches using antibodies to tumour associated antigens, and antibodies that deplete target cell types (e.g., unconjugated anti-CD20 antibodies such as Rituximab, radiolabeled anti-CD20 antibodies Bexxar and Zevalin, and anti-CD54 antibody Campath); approaches using anti-idiotypic antibodies; approaches that enhance Natural Killer cell function; and approaches that utilize antibody-toxin conjugates (e.g. anti-CD33 antibody Mylotarg); immunotoxins such as moxetumumab pasudotox; agonists of toll-like receptor 7 or toll-like receptor 9;
iv. Efficacy enhancers, such as leucovorin.

In one embodiment there is provided a compound of Formula (I), or a pharmaceutically acceptable salt thereof, for use in the treatment of cancer, where the compound of Formula (I), or a pharmaceutically acceptable salt thereof, is administered in combination with at least one additional anti-tumour substance. In one embodiment there is one additional anti-tumour substance. In one embodiment there are two additional anti-tumour substances. In one embodiment there are three or more additional anti-tumour substances. In any embodiment the additional anti-tumour substance is selected from one or more of the anti-tumour substances listed under points (i)-(iv) above.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with at least one additional anti-tumour substance. In one embodiment there is one additional anti-tumour substance. In one embodiment there are two additional anti-tumour substances. In one embodiment there are three or more additional anti-tumour substances. In any embodiment the additional anti-tumour substance is selected from one or more of the anti-tumour substances listed under points (i)-(iv) above.

In one embodiment there is provided a method of treating cancer in a warm-blooded animal who is in need of such treatment, which comprises administering to said warm-blooded animal a compound of Formula (I), or a pharmaceutically acceptable salt thereof and at least one additional anti-tumour substance, wherein the amounts of the compound of Formula (I), or a pharmaceutically acceptable salt thereof, and the additional anti-tumour substance are jointly effective in producing an anti-cancer effect. In any embodiment the additional anti-tumour substance is selected from one or more of the anti-tumour substances listed under points (i)-(iv) above.

In one embodiment there is provided a method of treating cancer in a warm-blooded animal who is in need of such treatment, which comprises administering to said warm-blooded animal a compound of Formula (I), or a pharmaceutically acceptable salt thereof, and simultaneously, separately or sequentially administering at least one additional anti-tumour substance to said warm-blooded animal, wherein the amounts of the compound of Formula (I), or pharmaceutically acceptable salt thereof, and the additional anti-tumour substance are jointly effective in producing an anti-cancer effect. In any embodiment the additional anti-tumour substance is selected from one or more of the anti-tumour substances listed under points (i)-(iv) above.

In one embodiment there is provided a compound of Formula (I), or a pharmaceutically acceptable salt thereof, and at least one anti-neoplastic agent for use in the treatment of cancer. In one embodiment there is provided a compound of Formula (I), or a pharmaceutically acceptable salt thereof, for use in the treatment of cancer, where the compound of Formula (I), or a pharmaceutically acceptable salt thereof, is administered in combination with at least one anti-neoplastic agent. In one embodiment the anti-neoplastic agent is selected from the list of antineoplastic agents in point (i) above.

In one embodiment there is provided a compound of Formula (I), or a pharmaceutically acceptable salt thereof, and at least one anti-neoplastic agent for use in the simultaneous, separate or sequential treatment of cancer. In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with at least one anti-neoplastic agent. In one embodiment the antineoplastic agent is selected from the list of antineoplastic agents in point (i) above.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with at least one additional anti-tumour substance selected from cisplatin, oxaliplatin, carboplatin, valrubicin, idarubicin, doxorubicin, pirarubicin, irinotecan, topotecan, amrubicin, epirubicin, etoposide, mitomycin, bendamustine, chlorambucil, cyclophosphamide, ifosfamide, carmustine, melphalan, bleomycin, olaparib, MEDI4736, AZD1775 and AZD6738.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with at least one additional anti-tumour substance selected from cisplatin, oxaliplatin, carboplatin, doxorubicin, pirarubicin, irinotecan, topotecan, amrubicin, epirubicin, etoposide, mitomycin, bendamustine, chlorambucil, cyclophosphamide, ifosfamide, carmustine, melphalan, bleomycin, olaparib, AZD1775 and AZD6738.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with at least one additional anti-tumour substance selected from doxorubicin, irinotecan, topotecan, etoposide, mitomycin, bendamustine, chlorambucil, cyclophosphamide, ifosfamide, carmustine, melphalan, bleomycin and olaparib.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with at least one additional anti-tumour substance selected from doxorubicin, irinotecan, topotecan, etoposide, mitomycin, bendamustine, chlorambucil, cyclophosphamide, ifosfamide, carmustine, melphalan and bleomycin.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with at least one additional anti-tumour substance selected from doxorubicin, pirarubicin, amrubicin and epirubicin.

In one embodiment there is provided a compound of Formula (I), or a pharmaceutically acceptable salt thereof, for use in the treatment of acute myeloid leukaemia, where the compound of Formula (I), or a pharmaceutically acceptable salt thereof, is administered simultaneously, separately or sequentially with at least one additional anti-tumour substance selected from doxorubicin, pirarubicin, amrubicin and epirubicin.

In one embodiment there is provided a compound of Formula (I), or a pharmaceutically acceptable salt thereof, for use in the treatment of breast cancer, where the compound of Formula (I), or a pharmaceutically acceptable salt thereof, is administered simultaneously, separately or sequentially with at least one additional anti-tumour substance selected from doxorubicin, pirarubicin, amrubicin and epirubicin.

In one embodiment there is provided a compound of Formula (I), or a pharmaceutically acceptable salt thereof, for use in the treatment of triple negative breast cancer, where the compound of Formula (I), or a pharmaceutically acceptable salt thereof, is administered simultaneously, separately or sequentially with at least one additional anti-tumour substance selected from doxorubicin, pirarubicin, amrubicin and epirubicin.

In one embodiment there is provided a compound of Formula (I), or a pharmaceutically acceptable salt thereof, for use in the treatment of hepatocellular carcinoma, where the compound of Formula (I), or a pharmaceutically acceptable salt thereof, is administered simultaneously, separately or sequentially with at least one additional anti-tumour substance selected from doxorubicin, pirarubicin, amrubicin and epirubicin.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with irinotecan.

In one embodiment there is provided a compound of Formula (I), or a pharmaceutically acceptable salt thereof, for use in the treatment of colorectal cancer, where the compound of Formula (I), or a pharmaceutically acceptable salt thereof, is administered simultaneously, separately or sequentially with irinotecan.

In one embodiment there is provided a compound of Formula (I), or a pharmaceutically acceptable salt thereof, for use in the treatment of colorectal cancer, where the compound of Formula (I), or a pharmaceutically acceptable salt thereof, is administered simultaneously, separately or sequentially with FOLFIRI.

FOLFIRI is a dosage regime involving a combination of leucovorin, 5-fluorouracil and irinotecan.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with olaparib.

In one embodiment there is provided a compound of Formula (I), or a pharmaceutically acceptable salt thereof, for use in the treatment of gastric cancer, where the compound of Formula (I), or a pharmaceutically acceptable salt thereof, is administered simultaneously, separately or sequentially with olaparib.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with topotecan.

In one embodiment there is provided a compound of Formula (I), or a pharmaceutically acceptable salt thereof, for use in the treatment of lung cancer, where the compound of Formula (I), or a pharmaceutically acceptable salt thereof, is administered simultaneously, separately or sequentially with topotecan.

In one embodiment there is provided a compound of Formula (I), or a pharmaceutically acceptable salt thereof, for use in the treatment of small cell lung cancer, where the compound of Formula (I), or a pharmaceutically acceptable salt thereof, is administered simultaneously, separately or sequentially with topotecan.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with immunotherapy. In one embodiment the immunotherapy is one or more of the agents listed under point (iii) above.

In one embodiment there is provided a compound of Formula (I), or a pharmaceutically acceptable salt thereof, for use in the treatment of cancer, where the compound of Formula (I), or a pharmaceutically acceptable salt thereof, is administered simultaneously, separately or sequentially with an anti-PD-L1 antibody (for example MEDI4736).

According to a further embodiment there is provided a kit comprising:

a) A compound of formula (I), or a pharmaceutically acceptable salt thereof, in a first unit dosage form;

b) A further additional anti-tumour substance in a further unit dosage form;

c) Container means for containing said first and further unit dosage forms; and optionally

d) Instructions for use. In one embodiment the anti-tumour substance comprises an anti-neoplastic agent.

In any embodiment where an anti-neoplastic agent is mentioned, the anti-neoplastic agent is one or more of the agents listed under point (i) above.

The compounds of Formula (I), and pharmaceutically acceptable salts thereof, may be administered as pharmaceutical compositions, comprising one or more pharmaceutically acceptable excipients.

Therefore, in one embodiment there is provided a pharmaceutical composition comprising a compound of Formula (I), or a pharmaceutically acceptable salt thereof, and at least one pharmaceutically acceptable excipient.

The pharmaceutically acceptable excipient(s) selected for inclusion in a particular composition will depend on factors such as the mode of administration and the form of the composition provided. Suitable pharmaceutically acceptable excipients are well known to persons skilled in the art and are described, for example, in the Handbook of Pharmaceutical Excipients, Sixth edition, Pharmaceutical Press, edited by Rowe, Ray C; Sheskey, Paul J; Quinn, Marian. Pharmaceutically acceptable excipients may function as, for example, adjuvants, diluents, carriers, stabilisers, flavourings, colorants, fillers, binders, disintegrants, lubricants, glidants, thickening agents and coating agents. As persons skilled in the art will appreciate, certain pharmaceutically acceptable excipients may serve more than one function and may serve alternative functions depending on how much of the excipient is present in the composition and what other excipients are present in the composition.

The pharmaceutical compositions may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing), or as a suppository for rectal dosing. The compositions may be obtained by conventional procedures well known in the art. Compositions intended for oral use may contain additional components, for example, one or more colouring, sweetening, flavouring and/or preservative agents.

The compound of Formula (I) will normally be administered to a warm-blooded animal at a unit dose within the range 2.5-5000 mg/m² body area of the animal, or to approximately 0.05-100 mg/kg, and this normally provides a therapeutically-effective dose. A unit dose form such as a tablet or capsule will usually contain, for example 0.1-250 mg of active ingredient. The daily dose will necessarily be varied depending upon the host treated, the particular route of administration, any therapies being co-administered, and the severity of the illness being treated. Accordingly the practitioner who is treating any particular patient may determine the optimum dosage.

The pharmaceutical compositions described herein comprise compounds of Formula (I), or a pharmaceutically acceptable salt thereof, and are therefore expected to be useful in therapy.

As such, in one embodiment there is provided a pharmaceutical composition for use in therapy, comprising a compound of Formula (I), or a pharmaceutically acceptable salt thereof, and at least one pharmaceutically acceptable excipient.

In one embodiment there is provided a pharmaceutical composition for use in the treatment of a disease in which inhibition of ATM kinase is beneficial, comprising a compound of Formula (I), or a pharmaceutically acceptable salt thereof, and at least one pharmaceutically acceptable excipient.

In one embodiment there is provided a pharmaceutical composition for use in the treatment of cancer, comprising a compound of Formula (I), or a pharmaceutically acceptable salt thereof, and at least one pharmaceutically acceptable excipient.

In one embodiment there is provided a pharmaceutical composition for use in the treatment of a cancer in which inhibition of ATM kinase is beneficial, comprising a compound of Formula (I), or a pharmaceutically acceptable salt thereof, and at least one pharmaceutically acceptable excipient.

In one embodiment there is provided a pharmaceutical composition for use in the treatment of colorectal cancer, glioblastoma, gastric cancer, ovarian cancer, diffuse large B-cell lymphoma, chronic lymphocytic leukaemia, acute myeloid leukaemia, head and neck squamous cell carcinoma, breast cancer, hepatocellular carcinoma, small cell lung cancer or non-small cell lung cancer, comprising a compound of Formula (I), or a pharmaceutically acceptable salt thereof, and at least one pharmaceutically acceptable excipient.

### EXAMPLES

The various embodiments of the invention are illustrated by the following Examples. The invention is not to be interpreted as being limited to the Examples. During the preparation of the Examples, generally:
i. Operations were carried out at ambient temperature, i.e. in the range of about 17 to 30° C. and under an atmosphere of an inert gas such as nitrogen unless otherwise stated;
ii. Evaporations were carried out by rotary evaporation or utilising Genevac equipment in vacuo and work-up procedures were carried out after removal of residual solids by filtration;
iii. Flash chromatography purifications were performed on an automated Armen Glider Flash: Spot II Ultimate (Armen Instrument, Saint-Ave, France) or automated Presearch combiflash companions using prepacked Merck normal phase Si60 silica cartridges (granulometry: 15-40 or 40-63 μm) obtained from Merck, Darmstad, Germany, silicycle silica cartridges or graceresolv silica cartridges;
iv. Preparative chromatography was performed on a Waters instrument (600/2700 or 2525) fitted with a ZMD or ZQ ESCi mass spectrometers and a Waters X-Terra or a Waters X-Bridge or a Waters SunFire reverse-phase column (C-18, 5 microns silica, 19 mm or 50 mm diameter, 100 mm length, flow rate of 40 mL/minute) using decreasingly polar mixtures of water (containing 1% NH₃) and acetonitrile or decreasingly polar mixtures of water (containing 0.1% formic acid) and acetonitrile as eluents;
v. Yields, where present, are not necessarily the maximum attainable;
vi. Structures of end-products of Formula (I) were confirmed by nuclear magnetic resonance (NMR) spectroscopy, with NMR chemical shift values measured on the delta scale. Proton magnetic resonance spectra were determined using a Bruker advance 700 (700 MHz), Bruker Avance 500 (500 MHz), Bruker 400 (400 MHz) or Bruker 300 (300 MHz) instrument; 19F NMR were determined at 282 MHz or 376 MHz; 13C NMR were determined at 75 MHz or 100 MHz; measurements were taken at around 20-30° C. unless otherwise specified; the following abbreviations have been used: s, singlet; d, doublet; t, triplet; q, quartet; m, multiplet; dd, doublet of doublets; ddd, doublet of doublet of doublet; dt, doublet of triplets; bs, broad signal;
vii. End-products of Formula (I) were also characterised by mass spectroscopy following liquid chromatography (LCMS); LCMS was carried out using an Waters Alliance HT (2790 & 2795) fitted with a Waters ZQ ESCi or ZMD ESCi mass spectrometer and an X Bridge 5 μm C-18 column (2.1×50 mm) at a flow rate of 2.4 mL/min, using a solvent system of 95% A+5% C to 95% B+5% C over 4 minutes, where A=water, B=methanol, C=1:1 methanol:water (containing 0.2% ammonium carbonate); or by using a Shimadzu UFLC or UHPLC coupled with DAD detector, ELSD detector and 2020 EV mass spectrometer (or equivalent) fitted with a Phenomenex Gemini-NX C18 3.0×50 mm, 3.0 μM column or equivalent (basic conditions) or a Shim pack XR-ODS 3.0×50 mm, 2.2 μM column or Waters BEH C18 2.1×50 mm, 1.7 μM column or equivalent using a solvent system of 95% D+5% E to 95% E+5% D over 4 minutes, where D=water (containing 0.05% TFA), E=Acetonitrile (containing 0.05% TFA) (acidic conditions) or a solvent system of 90% F+10% G to 95% G+5% F over 4 minutes, where F=water (containing 6.5 mM ammonium hydrogen carbonate and adjusted to pH10 by addition of NH₃), G=Acetonitrile (basic conditions);
viii. Intermediates were not generally fully characterised and purity was assessed by thin layer chromatographic, mass spectral, HPLC and/or NMR analysis;
ix. X-ray powder diffraction spectra were determined (using a Bruker D4 Analytical Instrument) by mounting a sample of the crystalline material on a Bruker single silicon crystal (SSC) wafer mount and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5418 angstroms. The collimated X-ray source was passed through an automatic variable divergence slit set at V20 and the reflected radiation directed through a 5.89 mm antiscatter slit and a 9.55 mm detector slit. The sample was exposed for 0.03 seconds per 0.00570° 2-theta increment (continuous scan mode) over the range 2 degrees to 40 degrees 2-theta in theta-theta mode. The running time was 3 minutes and 36 seconds. The instrument was equipped with a Position sensitive detector (Lynxeye). Control and data capture was by means of a Dell Optiplex 686 NT 4.0 Workstation operating with Diffrac+software;
x. Differential Scanning Calorimetry was performed on a TA Instruments Q1000 DSC. Typically, less than 5 mg of material contained in a standard aluminium pan fitted with a lid was heated over the temperature range 25° C. to 300° C. at a constant heating rate of 10° C. per minute. A purge gas using nitrogen was used at a flow rate 50 ml per minute
xi. The following abbreviations have been used: h=hour(s); r.t.=room temperature (˜18-25° C.); conc.=concentrated; FCC=flash column chromatography using silica; DCM=dichloromethane; DIPEA=diisopropylethylamine; DMA=N,N-dimethylacetamide; DMF=N,N-dimethylformamide; DMSO=dimethylsulfoxide; Et₂O=diethyl ether; EtOAc=ethyl acetate; EtOH=ethanol; K₂CO₃=potassium carbonate; MeOH=methanol; MeCN=acetonitrile; MTBE=Methyltertbutylether; MgSO₄=anhydrous magnesium sulphate; Na₂SO₄=anhydrous sodium sulphate; NH₃=ammonia; THF=tetrahydrofuran; sat.=saturated aqueous solution; and
xii. IUPAC names were generated using either “Canvas” or “IBIS”, AstraZeneca proprietary programs. As stated in the introduction, the compounds of the invention comprise an imidazo[4,5-c]quinolin-2-one core. However, in certain Examples the IUPAC name describes the core as an imidazo[5,4-c]quinolin-2-one. The imidazo[4,5-c]quinolin-2-one and imidazo[5,4-c]quinolin-2-one cores are nevertheless the same, with the naming convention different because of the peripheral groups.

### Example 1

### 8-[4-[3-(Dimethylamino)propoxy]phenyl]-1-isopropyl-3-methyl-imidazo[4,5-c]quinolin-2-one

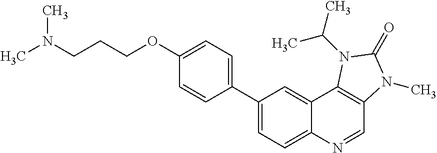

N,N-Dimethyl-3-[4-(4,4,5,5-tetramethyl-1,3,2-dioxaborolan-2-yl)phenoxy]propan-1-amine (60.6 mg, 0.20 mmol) and 8-bromo-1-isopropyl-3-methyl-imidazo[4,5-c]quinolin-2-one (53 mg, 0.17 mmol) were dissolved in dioxane (1.5 mL) then 2M K₂CO₃ (0.248 mL, 0.50 mmol) added and the solvent degassed. Dichloro[1,1′-bis(di-tert-butylphosphino)ferrocene]palladium(II) (5.39 mg, 0.0083 mmol) was added and the reaction heated to 90° C. for 30 minutes in a sealed vessel using the microwave reactor. The reaction was allowed to cool to ambient temperature, concentrated under reduced pressure and diluted with EtOAc (50 mL), washed sequentially with water (2×25 mL), and saturated brine (25 mL). The organic layer was dried with a phase separating cartridge and evaporated to afford crude product which was purified by FCC, elution gradient 0 to 10% MeOH in DCM followed by 10% MeOH:NH3 in DCM, to afford the desired material as a brown dry film (60.0 mg, 87%). NMR Spectrum: ¹H NMR (500 MHz, CDCl₃) δ 1.79 (6H, d), 2.01 (2H, dt), 2.28 (6H, s), 2.49 (2H, t), 3.58 (3H, s), 4.11 (2H, t), 5.27-5.38 (1H, m), 7.03-7.1 (2H, m), 7.59-7.66 (2H, m), 7.83 (1H, dd), 8.18 (1H, d), 8.32 (1H, s), 8.68 (1H, s). Mass Spectrum: m/z (ES+)[M+H]+=419.

The material could also be isolated as a methanesulfonic acid salt using the following procedure:

The isolated material (60 mg, 0.14 mmol) was dissolved in DCM (2 mL) and 1M methanesulfonic acid in DCM (0.135 mL, 0.14 mmol) was added. The solution was evaporated to dryness and dried in a vacuum oven for 4 h to afford the desired material as a methaesulfonic acid salt. NMR Spectrum: ¹H NMR (500 MHz, DMSO-d6) δ 1.70 (6H, d), 2.11-2.2 (2H, m), 2.32 (3H, s), 2.86 (6H, s), 3.27 (2H, s), 3.52 (3H, s), 4.15 (2H, t), 5.25-5.45 (1H, m), 7.14 (2H, d), 7.82 (2H, d), 7.97 (1H, d), 8.15 (1H, d), 8.39 (1H, d), 8.93 (1H, s), 9.35 (1H, s). Mass Spectrum: m/z (ES+)[M+H]+=419.

The following compounds could be prepared in an analogous fashion from the appropriate boronic ester and bromo intermediates.

| Example | Structure | Name |
|---|---|---|
| 2* | 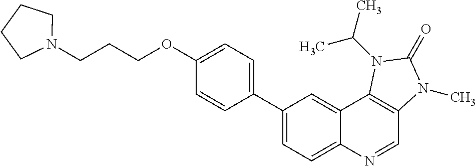 [SMILES: CC(C)n1c(=O)n(C)c2cnc3ccc(-c4ccc(OCCCN5CCCC5)cc4)cc3c21] | 1-isopropyl-3-methyl-8-[4-(3- pyrrolidin-1- ylpropoxy)phenyl]imidazo[4,5- c]quinolin-2-one |
| 3** | 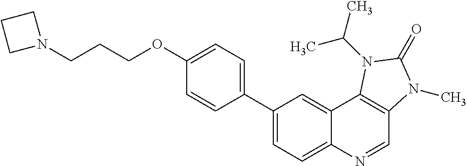 [SMILES: CC(C)n1c(=O)n(C)c2cnc3ccc(-c4ccc(OCCCN5CCC5)cc4)cc3c21] | 8-[4-[3-(azetidin-1- yl)propoxy]phenyl]-1- isopropyl-3-methyl- imidazo[4,5-c]quinolin-2-one |
| 4** | 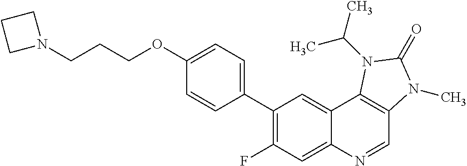 [SMILES: CC(C)n1c(=O)n(C)c2cnc3cc(F)c(-c4ccc(OCCCN5CCC5)cc4)cc3c21] | 8-[4-[3-(azetidin-1- yl)propoxy]phenyl]-7-fluoro-1- isopropyl-3-methyl- imidazo[4,5-c]quinolin-2-one |

*The reaction was performed with a 1:2 mixture of sodium tetrachloropalladate and 3-(di-tert-butylphosphino)propane-1-sulfonic acid (0.05M in water) as the catalyst and ligand and K₂CO₃ as the base and the reaction was stirred at 80° C. for 1 h.
**The catalyst used was chloro(2-dicyclohexylphosphino-2′,4′,6′-triisopropyl-1,1′-biphenyl)[2-(2′-amino-1,1′-biphenyl)]palladium(II) and the base used was Cs₂CO₃ and the reaction was heated at 80° C. for 4 h not using a microwave reactor. The material was purified using flash chromatography on a C18 column and the material was isolated as the free base.

### Example 2

(Free base) NMR Spectrum: ¹H NMR (500 MHz, CDCl₃) δ 1.59 (2H, s), 1.77-1.86 (10H, m), 2.06 (2H, dt), 2.55 (4H, s), 2.63-2.7 (2H, m), 3.59 (3H, s), 4.12 (2H, t), 5.30 (1H, s), 7.03-7.11 (2H, m), 7.59-7.66 (2H, m), 7.83 (1H, dd), 8.18 (1H, d), 8.32 (1H, s), 8.68 (1H, s). (Methane sulfonic acid salt) NMR Spectrum: ¹H NMR (500 MHz, DMSO-d6) δ 1.68 (6H, d), 1.89 (2H, dd), 2.04 (2H, t), 2.1-2.19 (2H, m), 2.31 (3H, s), 2.99-3.13 (2H, m), 3.35 (3H, s), 3.50 (3H, s), 3.60 (2H, d), 4.14 (2H, t), 5.33 (1H, p), 7.09-7.16 (2H, m), 7.77-7.83 (2H, m), 7.96 (1H, dd), 8.13 (1H, d), 8.37 (1H, d), 8.92 (1H, s), 9.50 (1H, s). Mass Spectrum: m/z (ES+)[M+H]+=445.

### Example 3

NMR Spectrum: ¹H NMR (400 MHz, MeOH-d4) δ 1.80 (6H, d), 1.97-2.07 (2H, m), 2.33-2.41 (2H, m), 3.09-3.14 (2H, m), 3.61 (3H, s), 3.79-3.84 (4H, m), 4.15 (2H, t), 5.36-5.48 (1H, m), 7.13 (2H, d), 7.75 (2H, d), 7.95 (1H, d), 8.15 (1H, d), 8.44 (1H, s), 8.80 (1H, s). Mass Spectrum: m/z (ES+)[M+H]+=431.

### Example 4

NMR Spectrum: ¹H NMR (400 MHz, MeOH-d4) δ 1.74 (6H, d), 1.92-2.04 (2H, m), 2.26-2.38 (2H, m), 3.01 (2H, t), 3.58 (3H, s), 3.70 (4H, t), 4.13 (2H, t), 5.24-5.38 (1H, m), 7.06-7.14 (2H, m), 7.61 (2H, d), 7.77 (1H, d), 8.29 (1H, d), 8.78 (1H, s). Mass Spectrum: m/z (ES+)[M+H]+=449.

The boronic acids described above were prepared as follows:

### N,N-Dimethyl-3-[4-(4,4,5,5-tetramethyl-1,3,2-dioxaborolan-2-yl)phenoxy]propan-1-amine

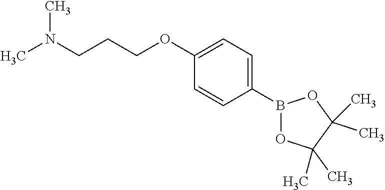

N,N-Dimethyl-3-[4-(4,4,5,5-tetramethyl-1,3,2-dioxaborolan-2-yl)phenoxy]propan-1-amine is available commercially from several suppliers including Apollo Scientific Ltd., Whitefield Rd, Bredbury, Stockport, Cheshire, SK6 2QR, UK. CAS number [627899-90-5], catalogue number OR12268. Alternatively, it can be prepared as follows:

A 1:1 complex of [1,1′-bis(diphenylphosphino)ferrocene]dichloropalladium(II) with dichloromethane (8.64 mg, 10.58 μmol) was added to 3-(4-bromophenoxy)-N,N-dimethylpropan-1-amine (546 mg, 2.12 mmol), 4,4,4′,4′,5,5,5′,5′-octamethyl-2,2′-bi(1,3,2-dioxaborolane) (644 mg, 2.54 mmol) and potassium acetate (830 mg, 8.46 mmol) in 1,4-dioxane (6 mL) warmed to 90° C. under nitrogen. The resulting suspension was stirred at 90° C. for 16 h. The reaction mixture was evaporated to dryness and re-dissolved in DCM (25 mL), and washed with water (20 mL). The organic layer was dried with a phase separating cartridge, filtered and evaporated to afford crude product. The crude product was purified by FCC, elution gradient 0 to 10% MeOH in DCM. Pure fractions were evaporated to dryness to afford the desired material as a brown waxy solid (274 mg, 42.4%). NMR Spectrum: ¹H NMR (500 MHz, CDCl₃) δ 1.33 (12H, s), 1.89-2.08 (2H, m), 2.32 (6H, s), 2.53 (2H, dt), 4.05 (2H, t), 6.86-6.91 (2H, m), 7.71-7.76 (2H, m). Mass Spectrum: m/z (ES+)[M+H]+=258.

### 3-(4-Bromophenoxy)-N,N-dimethyl-propan-1-amine

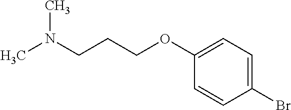

Di-tert-butyl azodicarboxylate (639 mg, 2.77 mmol) was added dropwise to a suspension of 4-bromophenol (400 mg, 2.31 mmol), 3-(dimethylamino)propan-1-ol (0.328 mL, 2.77 mmol) and triphenylphosphine (728 mg, 2.77 mmol) in DCM (3 mL) at 0° C. then the mixture was allowed to warm to ambient temperature and stirred for 3 h. The reaction mixture was purified by ion exchange chromatography, using an SCX column and eluting with 1M NH3/MeOH. The desired material was further purified by FCC, elution gradient 0 to 10% MeOH in DCM, to afford the desired material as a colourless oil (336 mg, 56.3%). NMR Spectrum: ¹H NMR (500 MHz, CDCl₃) δ 1.94 (2H, dq), 2.25 (6H, s), 2.4-2.47 (2H, m), 3.98 (2H, t), 6.74-6.82 (2H, m), 7.31-7.39 (2H, m). Mass Spectrum: m/z (ES+)[M+H]+=258.

### 1-[3-[4-(4,4,5,5-Tetramethyl-1,3,2-dioxaborolan-2-yl)phenoxy]propyl]pyrrolidine

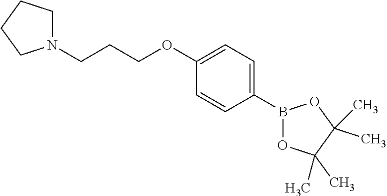 [SMILES: CC1(C)OB(c2ccc(OCCCN3CCCC3)cc2)OC1(C)C]

Potassium acetate (1.036 g, 10.56 mmol) was added to 1-(3-(4-bromophenoxy)propyl)pyrrolidine (1 g, 3.52 mmol), 4,4,4′,4′,5,5,5′,5′-octamethyl-2,2′-bi(1,3,2-dioxaborolane) (1.072 g, 4.22 mmol) and [1,1′-bis(diphenylphosphino)ferrocene]dichloropalladium(II) (0.129 g, 0.18 mmol) in 1,4-dioxane (1 mL) at 25° C. under nitrogen. The resulting mixture was stirred at 100° C. for 3 h. The solvent was removed under reduced pressure. The crude product was purified by FCC, elution gradient 0 to 10% MeOH in DCM. Pure fractions were evaporated to dryness to afford the desired material as a brown oil (1.100 g, 94%). Mass Spectrum: m/z (ES+)[M+H]+ 332.

1-[3-[4-(4,4,5,5-Tetramethyl-1,3,2-dioxaborolan-2-yl)phenoxy]propyl]pyrrolidine can also be prepared as follows:

Diisopropylazodicarboxylate (6.71 mL, 34.08 mmol) was added dropwise to 4-(4,4,5,5-tetramethyl-1,3,2-dioxaborolan-2-yl)phenol (5.00 g, 22.72 mmol), triphenylphosphine (8.94 g, 34.08 mmol) and 3-(pyrrolidin-1-yl)propan-1-ol (4.40 g, 34.08 mmol) in THF (50 mL) at 0° C. under nitrogen. The resulting mixture was allowed to warm up to room temperature and stirred for 18 h. The reaction mixture was evaporated to afford yellow oil. The yellow oil was triturated from heptane/EtOAc (80/20), and the white solid was filtered off. The filtrate was concentrated and the crude product was purified by FCC, elution gradient 0 to 100% EtOAc in heptane. Pure fractions were evaporated to dryness to afford the desired material as a pale yellow gum (2.05 g, 27%).

¹H NMR (500 MHz, DMSO-d6) δ 1.13 (2H, t), 1.28 (12H, s), 1.68 (4H, dq), 1.79-1.96 (2H, m), 2.46-2.56 (4H, m), 3.94-4.11 (2H, m), 6.83-6.97 (2H, m), 7.58-7.66 (2H, m). Mass Spectrum: m/z (ES+)[M+H]+ not observed.

### 1-[3-(4-Bromophenoxy)propyl]pyrrolidine

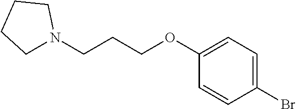 [SMILES: Brc1ccc(OCCCN2CCCC2)cc1]

A mixture of 1-(3-chloropropyl)pyrrolidine, hydrochloride salt (1.5 g, 8.15 mmol), 4-bromophenol (1.410 g, 8.15 mmol) and K₂CO₃ (4.50 g, 32.59 mmol) in DMF (15 mL) was heated to 90° C. for 18 h. The reaction mixture was cooled to ambient temperature, diluted with EtOAc (300 mL), washed with water (200 mL), saturated brine (200 mL), dried over a phase separator and the solvent was removed under reduced pressure to afford crude product. The crude product was purified by ion exchange chromatography, using an SCX column and eluting with 1M NH3/MeOH, to afford the desired material as a brown oil (1.97 g, 85%). NMR Spectrum: 1H NMR (500 MHz, CDCl₃) δ 1.73-1.85 (4H, m), 1.94-2.04 (2H, m), 2.49-2.56 (4H, m), 2.57-2.64 (2H, m), 3.99 (2H, t), 6.75-6.81 (2H, m), 7.31-7.39 (2H, m). Mass Spectrum: m/z (ES+)[M+H]+=286.

### 1-[3-[4-(4,4,5,5-Tetramethyl-1,3,2-dioxaborolan-2-yl)phenoxy]propyl]azetidine

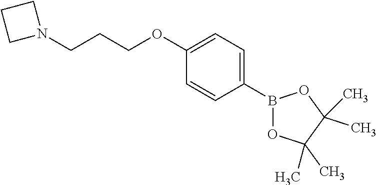 [SMILES: CC1(C)OB(c2ccc(OCCCN3CCC3)cc2)OC1(C)C]

1,1′-Bis(diphenylphosphino)ferrocenedichloropalladium(II) (0.387 g, 0.53 mmol) was added to 1-[3-(4-bromophenoxy)propyl]azetidine (1.43 g, 5.29 mmol), 4,4,4′,4′,5,5,5′,5′-octamethyl-2,2′-bi(1,3,2-dioxaborolane) (1.613 g, 6.35 mmol) and potassium acetate (1.039 g, 10.59 mmol) in 1,4-dioxane (40 mL) under nitrogen. The resulting mixture was stirred at 90° C. overnight. The solution was cooled to room temperature then used directly in the next step without further work-up of purification.

### 1-[3-(4-Bromophenoxy)propyl]azetidine

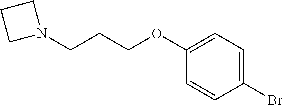 [SMILES: Brc1ccc(OCCCN2CCC2)cc1]

Di-tert-butyl azodicarboxylate (1.996 g, 8.67 mmol) was added to a stirred mixture of 4-bromophenol (1 g, 5.78 mmol), 3-(azetidin-1-yl)propan-1-ol (0.999 g, 8.67 mmol) and triphenylphosphine (2.274 g, 8.67 mmol) in DCM (20 mL) under nitrogen and the resulting mixture stirred at ambient temperature for 4 h. The solvent was removed under reduced pressure and the crude product purified by FCC, elution gradient 2 to 10% MeOH in DCM, to afford the desired material as a yellow oil (1.43 g, 92%). Mass Spectrum: m/z (ES+)[M+H]+=270

### 3-(Azetidin-1-yl)propan-1-ol

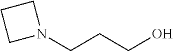

A solution of lithium aluminium hydride (2.0 M in THF) (8.38 mL, 16.76 mmol) diluted in further THF (20 mL) was added to a mixture of methyl 3-(azetidin-1-yl)propanoate (2 g, 13.97 mmol) in THF (5 mL) dropwise at 0° C. under an inert atmosphere. The resulting solution was stirred at 0° C. for 1 h then the reaction mixture treated with sodium sulphate decahydrate and stirred for 30 minutes. The solid was removed by filtration and discarded and the filtrate evaporated to afford the desired material (1.240 g, 77%) as a colourless oil. NMR Spectrum: ¹H NMR (400 MHz, CDCl₃) δ 1.51-1.57 (2H, m), 2-2.07 (2H, m), 2.6-2.66 (2H, m), 3.20 (4H, t), 3.7-3.76 (2H, m).

### Methyl 3-(azetidin-1-yl)propanoate

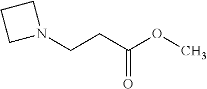

Methyl acrylate (2.082 ml, 23.12 mmol) was added to a solution of azetidine (1.2 g, 21.02 mmol) in DCM and the resulting solution stirred at ambient temperature, under an inert atmosphere for 16 h. The reaction mixture was evaporated and the crude product purified by FCC, eluted with 25% EtOAc in DCM, to afford the desired material (2.0 g, 66.5%) as a colourless oil. NMR Spectrum: ¹H NMR (400 MHz, CDCl₃) δ 1.97-2.1 (2H, m), 2.33 (2H, d), 2.67 (2H, d), 3.18 (4H, t), 3.67 (3H, s).

The bromo intermediates described above were prepared as follows:

### Intermediate A1: 8-Bromo-7-fluoro-1-isopropyl-3-methyl-imidazo[4,5-c]quinolin-2-one

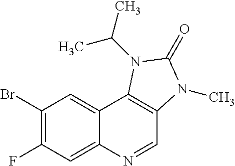 [SMILES: CC(C)n1c(=O)n(C)c2cnc3cc(F)c(Br)cc3c21]

A solution of sodium hydroxide (11.29 g, 282.28 mmol) in water (600 mL) was added to a stirred mixture of 8-bromo-7-fluoro-1-isopropyl-3H-imidazo[4,5-c]quinolin-2-one (61 g, 188.19 mmol), tetrabutylammonium bromide (6.07 g, 18.82 mmol) and methyl iodide (23.53 mL, 376.37 mmol) in DCM (1300 mL) and the mixture stirred at ambient temperature for 17 h. The same process was repeated on an identical scale and the reaction mixtures combined, concentrated and diluted with MeOH (750 mL). The precipitate was collected by filtration, washed with MeOH (500 mL) and the solid dried under vacuum to afford the desired material as a white solid (108 g, 85%). NMR Spectrum: ¹H NMR (400 MHz, CDCl₃) δ 1.76 (6H, d), 3.57 (3H, s), 5.13 (1H, t), 7.83 (1H, d), 8.41 (1H, d), 8.69 (1H, s). Mass Spectrum: m/z (ES+)[M+H]+=380.

### Intermediate A2: 8-Bromo-7-fluoro-1-isopropyl-3H-imidazo[4,5-c]quinolin-2-one

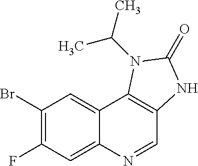 [SMILES: CC(C)n1c(=O)[nH]c2cnc3cc(F)c(Br)cc3c21]

Triethylamine (164 mL, 1173.78 mmol) was added in one portion to 6-bromo-7-fluoro-4-(isopropylamino)quinoline-3-carboxylic acid (128 g, 391.26 mmol) in DMF (1500 mL) and the mixture stirred at ambient temperature under an inert atmosphere for 30 minutes. Diphenylphosphoryl azide (101 mL, 469.51 mmol) was added and the solution stirred for a further 30 minutes at ambient temperature then 3 h at 60° C. The reaction mixture was poured into ice water, the precipitate collected by filtration, washed with water (1 L) and dried under vacuum to afford the desired material as a yellow solid (122 g, 96%). NMR Spectrum: ¹H NMR (400 MHz, DMSO-d6) δ 1.62 (6H, d), 5.12-5.19 (1H, m), 7.92 (1H, d), 8.57 (1H, d), 8.68 (1H, s), 11.58 (1H, s). Mass Spectrum: m/z (ES+)[M+H]+=324.

### Intermediate A3: 6-Bromo-7-fluoro-4-(isopropylamino)quinoline-3-carboxylic acid

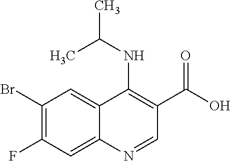 [SMILES: CC(C)Nc1c(C(=O)O)cnc2cc(F)c(Br)cc12]

2N Sodium hydroxide solution (833 mL, 1666.66 mmol) was added portionwise to ethyl 6-bromo-7-fluoro-4-(isopropylamino)quinoline-3-carboxylate (148 g, 416.66 mmol) in THF (1500 mL) at 15° C. and the resulting mixture stirred at 60° C. for 5 h. The reaction mixture was concentrated, diluted with water (2 L) and the mixture acidified with 2M hydrochloric acid. The precipitate was collected by filtration, washed with water (1 L) and dried under vacuum to afford the desired material as a white solid (128 g, 94%). NMR Spectrum: ¹H NMR (400 MHz, DMSO-d6) δ 1.24-1.36 (6H, m), 4.37 (1H, s), 7.78 (1H, t), 8.55 (1H, s), 8.90 (1H, s). Mass Spectrum: m/z (ES+)[M+H]+=327.

### Intermediate A4: Ethyl 6-bromo-7-fluoro-4-(isopropylamino)quinoline-3-carboxylate

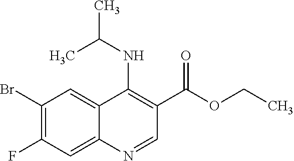 [SMILES: CCOC(=O)c1cnc2cc(F)c(Br)cc2c1NC(C)C]

DIPEA (154 mL, 884.07 mmol) was added portionwise to propan-2-amine (39.2 g, 663.05 mmol) and ethyl 6-bromo-4-chloro-7-fluoroquinoline-3-carboxylate (147 g, 442.04 mmol) in DMA (600 mL) at ambient temperature and the resulting mixture stirred at 100° C. for 4 h. The reaction mixture was poured into ice water, the precipitate collected by filtration, washed with water (1 L) and dried under vacuum to afford the desired material as a light brown solid (148 g, 94%). NMR Spectrum: ¹H NMR (400 MHz, DMSO-d6) δ 1.26-1.33 (9H, m), 4.17-4.25 (1H, m), 4.32-4.37 (2H, m), 7.28 (1H, d), 8.50 (1H, d), 8.59 (1H, d), 8.86 (1H, s). Mass Spectrum: m/z (ES+)[M+H]+=355.

### Intermediate A5: Ethyl 6-bromo-4-chloro-7-fluoroquinoline-3-carboxylate

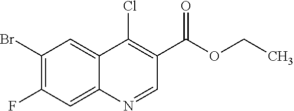 [SMILES: CCOC(=O)c1cnc2cc(F)c(Br)cc2c1Cl]

DMF (0.535 mL, 6.91 mmol) was added to ethyl 6-bromo-7-fluoro-1-[(4-methoxyphenyl)methyl]-4-oxo-quinoline-3-carboxylate (200 g, 460.56 mmol) in thionyl chloride (600 mL) at 10° C. under an inert atmosphere and the resulting mixture stirred at 70° C. for 3 h. The mixture was evaporated to dryness and the residue azeotroped with toluene (300 mL) to afford crude product. The crude product was purified by crystallisation from hexane to afford the desired material as a white solid (147 g, 96%). NMR Spectrum: ¹H NMR (400 MHz, CDCl₃) δ 1.49 (3H, t), 4.51-4.56 (2H, m), 7.91 (1H, d), 8.71 (1H, d), 9.26 (1H, s). Mass Spectrum: m/z (ES+)[M+H]+=334.

### Intermediate A6: Ethyl 6-bromo-7-fluoro-1-[(4-methoxyphenyl)methyl]-4-oxo-quinoline-3-carboxylate

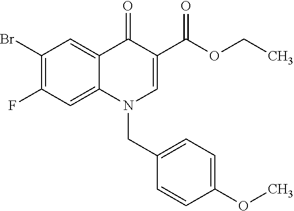 [SMILES: CCOC(=O)c1cn(Cc2ccc(OC)cc2)c2cc(F)c(Br)cc2c1=O]

DBU (76 mL, 506.32 mmol) was added slowly to ethyl-2-(5-bromo-2,4-difluoro-benzoyl)-3-[(4-methoxyphenyl)methylamino]prop-2-enoate (230 g, 506.32 mmol) in acetone (800 mL) at 10° C. over a period of 5 minutes under an inert atmosphere and the resulting mixture stirred at ambient temperature for 16 h. The precipitate was collected by filtration, washed with Et₂O (3×500 mL) and dried under vacuum to afford the desired material as a white solid (166 g, 75%). NMR Spectrum: ¹H NMR (400 MHz, DMSO-d6) δ 1.29 (3H, t), 3.72 (3H, s), 4.22-4.27 (21H, m), 5.57 (2H, s), 6.92-6.95 (2H, m), 7.24 (2H, d), 7.79 (1H, d), 8.40 (1H, d), 8.89 (1H, s). Mass Spectrum: m/z (ES+)[M+H]+=434.

### Intermediate A7: Ethyl-2-(5-bromo-2,4-difluoro-benzoyl)-3-[(4-methoxyphenyl)methylamino]prop-2-enoate

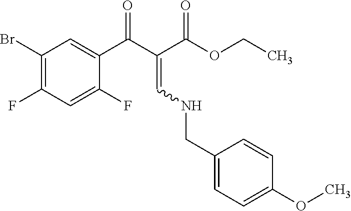 [SMILES: CCOC(=O)C(=CNCc1ccc(OC)cc1)C(=O)c1cc(Br)c(F)cc1F]

(E)-Ethyl 3-(dimethylamino)acrylate (80 mL, 555.50 mmol) was added dropwise to a mixture of DIPEA (132 mL, 757.50 mmol) and 5-bromo-2,4-difluoro-benzoyl chloride (129 g, 505.00 mmol) in toluene (600 mL) at ambient temperature under an inert atmosphere. The resulting solution was stirred at 70° C. for 17 h then allowed to cool. (4-Methoxyphenyl)methanamine (66.0 mL, 505.29 mmol) was added portionwise to the mixture and the reaction stirred for 3 h at ambient temperature. The reaction mixture was diluted with DCM (2 L), washed sequentially with water (4×200 mL), saturated brine (300 mL), the organic layer dried over Na₂SO₄, filtered and evaporated to afford the desired material as a light brown solid (230 g, 100%) which was used in the next step without further purification. NMR Spectrum: ¹H NMR (400 MHz, CDCl₃) δ 1.09 (3H, t), 3.82 (3H, s), 4.00-4.10 (2H, m), 4.55 (2H, t), 6.84-6.96 (3H, m), 7.20-7.29 (2H, m), 7.55 (1H, d), 8.18 (1H, t) Mass Spectrum: m/z (ES+)[M+H]+=454.

### Intermediate A8: 5-Bromo-2,4-difluoro-benzoyl chloride

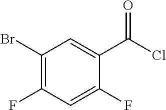 [SMILES: O=C(Cl)c1cc(Br)c(F)cc1F]

Thionyl chloride (55.4 mL, 759.50 mmol) was added portionwise to a mixture of DMF (7.84 mL, 101.27 mmol) and 5-bromo-2,4-difluorobenzoic acid (120 g, 506.33 mmol) in toluene (600 mL) at 15° C. over a period of 5 minutes under an inert atmosphere. The resulting mixture was stirred at 70° C. for 4 h then evaporated to dryness and the residue was azeotroped with toluene to afford the desired material as a brown oil (129 g, 100%) which was used directly in the next step without purification. NMR Spectrum: ¹H NMR (400 MHz, CDCl₃) δ 7.04-7.09 (1H, m), 8.34-8.42 (1H, m)

Intermediate A2 8-Bromo-7-fluoro-1-isopropyl-3H-imidazo[4,5-c]quinolin-2-one can also be prepared as described below:

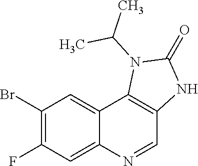 [SMILES: CC(C)n1c(=O)[nH]c2cnc3cc(F)c(Br)cc3c21]

1,3,5-Trichloro-1,3,5-triazinane-2,4,6-trione (5.91 g, 25.45 mmol) was added portionwise to a stirred suspension of 6-bromo-7-fluoro-4-(isopropylamino)quinoline-3-carboxamide (16.6 g, 50.89 mmol) and 1,8-diazabicyclo[5.4.0]undec-7-ene (15.22 mL, 101.79 mmol) in MeOH (200 mL) at 5° C. The resulting suspension was stirred at ambient temperature for 1 h. The reaction was filtered and the solid dried in a vacuum oven for 2 h to afford the desired material as a pale yellow solid (14.18 g, 86%). Additional material was obtained after leaving the filtrate to stand for 2 days and then filtering. The additional solid isolated was heated in EtOH (50 mL) for 30 minutes then allowed to cool and filtered to provide additional desired material as a white solid (2.6 mg). Analytical data was consistent with that obtained from alternative preparations described earlier.

### Intermediate A9: 6-Bromo-7-fluoro-4-(isopropylamino)quinoline-3-carboxamide

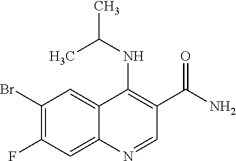 [SMILES: CC(C)Nc1c(C(N)=O)cnc2cc(F)c(Br)cc12]

Propan-2-amine (2.80 ml, 32.62 mmol) was added to a suspension of 6-bromo-4-chloro-7-fluoro-quinoline-3-carboxamide (10 g, 29.65 mmol) and K₂CO₃ (8.20 g, 59.31 mmol) in acetonitrile (250 mL) and the mixture stirred at 95° C. for 4 h. Further propan-2-amine (2 mL) was added and the mixture stirred at 95° C. for another 4 h then at ambient temperature overnight. Water was added to the mixture and the solid collected by filtration and dried under vacuum to afford the desired material (8.25 g, 85%). NMR Spectrum: ¹H NMR (500 MHz, DMSO-d6) δ 1.25 (6H, d), 4.17 (1H, d), 7.51 (1H, s), 7.69 (1H, d), 8.11 (2H, s), 8.61 (1H, s), 8.67 (1H, d). Mass Spectrum: m/z (ES+)[M+H]+=236.

### Intermediate A10: 6-Bromo-4-chloro-7-fluoro-quinoline-3-carboxamide

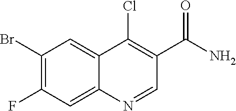 [SMILES: NC(=O)c1cnc2cc(F)c(Br)cc2c1Cl]

DMF (0.5 mL) was added to a stirred suspension of 6-bromo-7-fluoro-4-oxo-1H-quinoline-3-carboxylic acid (22.5 g, 78.66 mmol) in thionyl chloride (140 g, 1179.85 mmol) and the mixture heated to reflux for 2 h. The reaction was allowed to cool, concentrated in vacuo and the residue azeotroped twice with toluene to afford a yellow solid. This solid was added portionwise to a solution of ammonium hydroxide (147 mL, 1179.85 mmol) at 0° C. The white suspension was stirred for 15 minutes then the solid filtered, washed with water and dried under vacuum to afford the desired material (23.80 g, 100%) as a white powder. NMR Spectrum: ¹H NMR (400 MHz, DMSO-d6) δ 8.92 (1H, s), 8.59 (1H, d), 8.21 (1H, s), 8.09 (1H, d), 7.98 (1H, s). Mass Spectrum: m/z (ES+)[M+H]+=304.8.

### Intermediate A11: 6-Bromo-7-fluoro-4-oxo-1H-quinoline-3-carboxylic Acid

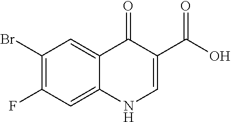 [SMILES: O=C(O)c1c[nH]c2cc(F)c(Br)cc2c1=O]

A solution of sodium hydroxide (18.34 g, 458.44 mmol) in water (100 mL) was added to a stirred suspension of ethyl 6-bromo-7-fluoro-4-oxo-1H-quinoline-3-carboxylate (28.8 g, 91.69 mmol) in EtOH (500 mL) at ambient temperature. The reaction mixture was then stirred at 75° C. for 2 h, allowed to cool and the pH adjusted to 4 using 2N hydrochloric acid. The precipitate was collected by filtration, washed with water and dried under vacuum to afford the desired material (23.30 g, 89%) as a white powder. NMR Spectrum: ¹H NMR (400 MHz, DMSO-d6) δ 14.78 (1H, s), 13.45 (1H, s), 8.93 (1H, s), 8.46 (1H, d), 7.70 (1H, d). Mass Spectrum: m/z (ES+)[M+H]+=287.8.

### Intermediate A12: Ethyl 6-bromo-7-fluoro-4-oxo-1H-quinoline-3-carboxylate

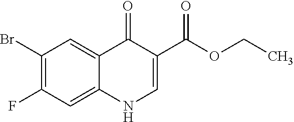 [SMILES: CCOC(=O)c1c[nH]c2cc(F)c(Br)cc2c1=O]

A solution of diethyl 2-[(4-bromo-3-fluoro-anilino)methylene]propanedioate (90 g, 249.88 mmol) in diphenyl ether (600 mL, 3.79 mol) was stirred at 240° C. for 2.5 h. The mixture was allowed to cool to 70° C., the solids collected by filtration and dried in a vacuum oven to afford the desired material (50 g, 64%) as a white solid which was used without further purification. NMR Spectrum: ¹H NMR (500 MHz, DMSO-d6, (100° C.)) δ 1.26-1.33 (3H, m), 4.25 (2H, q), 7.52 (1H, d), 8.37 (1H, d), 8.48 (1H, s), 12.05 (1H, s). Mass Spectrum: m/z (ES+)[M+H]+=314.

### Intermediate A13: Diethyl 2-[(4-bromo-3-fluoro-anilino)methylene]propanedioate

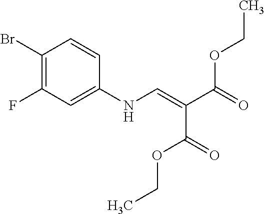 [SMILES: CCOC(=O)C(=CNc1ccc(Br)c(F)c1)C(=O)OCC]

A solution of 4-bromo-3-fluoroaniline (56.6 g, 297.87 mmol) and 1,3-diethyl 2-(ethoxymethylidene)propanedioate (72.45 g, 335.06 mmol) in EtOH (560 mL) was stirred at 80° C. for 4 h. The reaction mixture was allowed to cool, the solids collected by filtration and dried in an oven to afford the desired material (90 g, 84%) as an off-white solid which was used without further purification. NMR Spectrum: ¹H NMR (400 MHz, DMSO-d6) δ 1.26 (6H, q), 4.14 (2H, q), 4.22 (2H, q), 7.18-7.25 (1H, m), 7.57 (1H, dd), 7.64-7.7 (1H, m), 8.33 (1H, d), 10.62 (1H, d). Mass Spectrum: m/z (ES+)[M+H]+=360.

### Intermediate B1: 8-Bromo-1-isopropyl-3-methyl-imidazo[4,5-c]quinolin-2-one

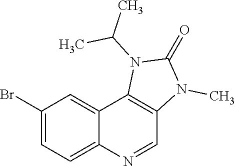 [SMILES: CC(C)n1c(=O)n(C)c2cnc3ccc(Br)cc3c21]

N,N-Dimethylformamide dimethyl acetal (54.2 mL, 408.29 mmol) was added to a solution of 8-bromo-1-isopropyl-3H-imidazo[4,5-c]quinolin-2-one (25.00 g, 81.66 mmol) in DMF (375 mL). The mixture was heated to 80° C. for 3 h then allowed to cool to ambient temperature and stirred for 16 h. The precipitate was collected by filtration, washed with water (4×300 mL) and dried under vacuum at 50° C. to afford the desired material as a white solid (23.82 g, 91%). NMR Spectrum: ¹H NMR (500 MHz, DMSO-d6) δ 1.63 (6H, d), 3.49 (3H, s), 5.15-5.23 (1H, m), 7.75 (1H, dd), 7.99 (1H, d), 8.44 (1H, d), 8.91 (1H, s). Mass Spectrum: m/z (ES+)[M+H]+=320.

### Intermediate B2: 8-Bromo-1-isopropyl-3H-imidazo[4,5-c]quinolin-2-one

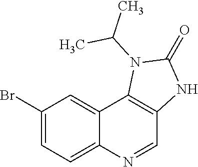 [SMILES: CC(C)n1c(=O)[nH]c2cnc3ccc(Br)cc3c21]

Triethylamine (45.3 mL, 332.06 mmol) was added to 6-bromo-4-(isopropylamino)quinoline-3-carboxylic acid (34.22 g, 110.69 mmol) in DMF (342 mL) at ambient temperature. After stirring at ambient temperature for 30 minutes, diphenyl phosphorazidate (26.2 mL, 121.76 mmol) was added and the resulting mixture stirred at 60° C. for 2 h. The reaction mixture was poured into water (1500 mL); the precipitate collected by filtration, washed with water (2×700 mL) and dried under vacuum at 50° C. to afford the desired material as a beige solid (29.6 g, 87%), which was used without further purification. NMR Spectrum: ¹H NMR (500 MHz, CDCl₃) δ 1.64 (6H, d), 5.06-5.21 (1H, m), 7.75 (1H, d), 7.98 (1H, d), 8.43 (1H, s), 8.69 (1H, s), 11.57 (1H, s). Mass Spectrum: m/z (ES+)[M+H]+=306.

### Intermediate B3: 6-Bromo-4-(isopropylamino)quinoline-3-carboxylic Acid

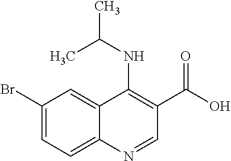 [SMILES: CC(C)Nc1c(C(=O)O)cnc2ccc(Br)cc12]

Ethyl 6-bromo-4-(isopropylamino)quinoline-3-carboxylate (38.0 g, 112.69 mmol) was suspended in MeOH (800 mL) and water (200 mL). 10M sodium hydroxide solution (33.8 mL, 338.07 mmol) was added and the mixture stirred at ambient temperature for 1 h. THF (200 mL) was added and the resultant mixture stirred for 16 h. Water (400 mL) was added and the organics removed under reduced pressure. The resulting aqueous solution was acidified to pH 4-5 with 2M HCl and the precipitate collected by filtration, washed with water and dried under vacuum to afford the desired material as a white solid (34.7 g, 100%). NMR Spectrum: 1H NMR (500 MHz, DMSO-d6) δ 1.33 (6H, d), 4.39 (1H, s), 7.78 (1H, d), 7.92 (1H, dd), 8.38 (1H, d), 8.88 (1H, s). Mass Spectrum: m/z (ES+)[M+H]+=309.

### Intermediate B4: Ethyl 6-bromo-4-(isopropylamino)quinoline-3-carboxylate

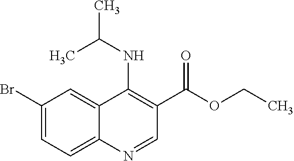 [SMILES: CCOC(=O)c1cnc2ccc(Br)cc2c1NC(C)C]

Propan-2-amine (11.00 ml, 128.02 mmol) was added to a suspension of ethyl 6-bromo-4-chloroquinoline-3-carboxylate (36.61 g, 116.38 mmol) and K₂CO₃ (32.2 g, 232.77 mmol) in acetonitrile (250 mL) at 0° C. The mixture was stirred at 54° C. under reflux for 3 h. Further K₂CO₃ (10.7 g, 77.6 mmol) and propan-2-amine (3.6 ml, 42.7 mmol) were added and stirring continued at 48° C. for a further 16 h. The solvents were removed in vacuo and the resulting residue partitioned between DCM (400 mL) and water (500 mL). The aqueous layer was re-extracted with DCM (2×200 mL); the combined organic layers were passed through a phase separating paper and concentrated under reduced pressure to afford the desired material as a beige solid (38.6 g, 98%). NMR Spectrum: ¹H NMR (500 MHz, CDCl₃) δ 1.40 (6H, d), 1.43 (3H, t), 4.32-4.37 (1H, m), 4.40 (2H, q), 7.72 (1H, dd), 7.81 (1H, d), 8.29 (1H, d), 8.95 (1H, d), 9.10 (1H, s). Mass Spectrum: m/z (ES+)[M+H]+=337.

### Intermediate B5: Ethyl 6-bromo-4-chloroquinoline-3-carboxylate

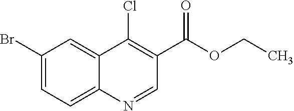 [SMILES: CCOC(=O)c1cnc2ccc(Br)cc2c1Cl]

DMF (0.119 mL, 1.54 mmol) was added to ethyl 6-bromo-1-[(4-methoxyphenyl)methyl]-4-oxoquinoline-3-carboxylate (160 g, 384.37 mmol) in thionyl chloride (800 mL) at ambient temperature under air. The resulting mixture was stirred at 75° C. for 16 h then the solvent removed under reduced pressure. The resulting mixture was azeotroped twice with toluene then n-hexane (500 mL) added. The precipitate was collected by filtration, washed with n-hexane (200 mL) and dried under vacuum to afford the desired material (100 g, 83%) as a brown solid. NMR Spectrum: ¹H NMR (400 MHz, CDCl₃) δ 1.47 (3H, t), 4.51 (2H, q), 7.95 (1H, dd), 8.11 (1H, d), 8.60 (1H, d), 9.24 (1H, s). Mass Spectrum: m/z (ES+)[M+H]+=314, 316.

On a larger scale, ethyl 6-bromo-1-[(4-methoxyphenyl)methyl]-4-oxoquinoline-3-carboxylate (5765 g, 13.85 mol) was charged to the vessel with thionyl chloride (28.8 L). An exotherm from 20-26° C. was observed. DMF (4.4 mL) was added with no observed exotherm and the batch heated to 75° C. and stirred for 17 h. HPLC showed 1.3% starting material remained with 98.0% product. The reaction was concentrated in vacuo and the residue azeotroped with toluene (25 L). The resulting solid was then slurried in heptane (18.5 L) for 2.5 h, filtered and washed with heptane (3×4 L). The solid was dried under vacuum at 35° C. to give 4077 g of the desired material (93% crude yield) which contained ˜5% of ethyl 6-bromo-1-[(4-methoxyphenyl)methyl]-4-oxoquinoline-3-carboxylate in addition to ˜4% hydrolysis product by HPLC (90% pure). The crude material (4077 g) was returned to the vessel and reprocessed with thionyl chloride (14.5 L) and DMF (2.2 mL). The mixture was heated to 75° C. for 40 h. The thionyl chloride was removed in vacuo and the residue azeotroped with toluene (10 L). The residue was slurried in heptane (18 L) for ˜16 h at 20° C. The solid was collected by filtration, one portion being filtered under nitrogen and washed with heptane (3 L) to yield 2196 g of desired material (90% NMR assay, 99% by HPLC). The remainder of the batch was filtered under air and washed with heptane (3 L) to yield 1905 g of the desired material (88% NMR assay, 99% by HPLC). The yellow solids were combined for further processing (4101 g, 3653 g active, 83% yield, 99% by HPLC).

### Intermediate B6: Ethyl 6-bromo-1-[(4-methoxyphenyl)methyl]-4-oxoquinoline-3-carboxylate

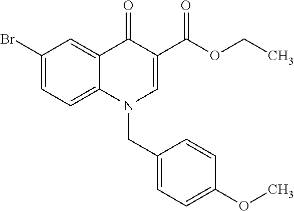 [SMILES: CCOC(=O)c1cn(Cc2ccc(OC)cc2)c2ccc(Br)cc2c1=O]

DBU (102 mL, 679.62 mmol) was added drop-wise to ethyl 2-(5-bromo-2-fluorobenzoyl)-3-[(4-methoxyphenyl)methylamino]prop-2-enoate (296.5 g, 679.62 mmol), in acetone (1.2 L) at ambient temperature over a period of 2 minutes. The resulting solution was stirred for 16 h then the solid removed by filtration and washed with MTBE to afford the desired material (180 g, 64%) as light yellow solid. NMR Spectrum: ¹H NMR (400 MHz, DMSO-d6) δ 1.30 (3H, t), 3.71 (3H, s), 4.25 (2H, q), 5.60 (2H, s), 6.90-6.95 (2H, m), 7.12-7.25 (2H, m), 7.67 (1H, d), 7.80-7.90 (1H, m), 8.30 (1H, d), 8.92 (1H, s). Mass Spectrum: m/z (ES+)[M+H]+=418.

On a larger scale, ethyl 2-(5-bromo-2-fluorobenzoyl)-3-[(4-methoxyphenyl)methylamino]prop-2-enoate (8434 g, (7730 g assumed active), 17.71 mol) was charged to the vessel with acetone (23.2 L) at 15° C. DBU (2.8 L, 18.72 mol) was added over 25 minutes with an observed exotherm from 18-23° C. over the addition. A precipitate formed after ˜25 minutes and the batch continued to exotherm reaching a maximum of 37° C. after 1 h. The reaction was stirred at 20° C. for 16.5 h at which point HPLC indicated consumption of starting material and 96.5% product. The resulting precipitate was collected by filtration washing with TBME (4×3.4 L). The solid was then dried under vacuum at 40° C. to give 6033 g of the desired material as a white solid (81.6% yield over 3 steps, 99.8% purity by HPLC). Analytical data was consistent with that obtained on previous batches.

### Intermediate B7: Ethyl 2-(5-bromo-2-fluorobenzoyl)-3-[(4-methoxyphenyl)methylamino]prop-2-enoate

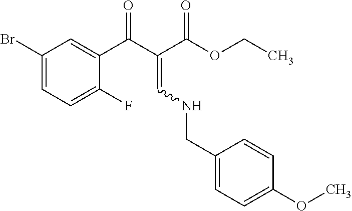 [SMILES: CCOC(=O)C(=CNCc1ccc(OC)cc1)C(=O)c1cc(Br)ccc1F]

(E)-Ethyl 3-(dimethylamino)acrylate (98 g, 685.00 mmol) was added portion-wise to 5-bromo-2-fluorobenzoyl chloride (163 g, 685 mmol) and DIPEA (120 mL, 685.00 mmol) in toluene (800 mL) at 10° C. over a period of 10 minutes. The resulting solution was stirred at 70° C. for 16 h then allowed to cool. (4-Methoxyphenyl)methanamine (94 g, 685 mmol) was added to the mixture over a period of 20 minutes at ambient temperature. The resulting solution was stirred for 3 h then the reaction mixture diluted with DCM (4 L), and washed with water (3×1 L). The organic phase was dried over Na₂SO₄, filtered and evaporated to give the desired material (300 g, 100%) as brown oil, which was used immediately in the subsequent reaction without further purification. Mass Spectrum: m/z (ES+)[M+H]+=436.

On a larger scale, 5-bromo-2-fluorobenzoyl chloride (4318 g, 4205 g active, 17.71 mol) was charged to the vessel as a solution in toluene (7.5 L). DIPEA (3150 mL, 18.08 mol) was added with no observed exotherm. Ethyl-3-(dimethylamino)acrylate (2532 g, 17.71 mol) was added portionwise over 30 minutes maintaining a batch temperature <40° C. An exotherm from 21-24° C. was noted over the 30 minute addition with a further slow rise to 38° C. over 1 h. The reaction was stirred at 20-30° C. for 16.5 h. 4-Methoxybenzylamine (2439 g, 17.78 mol) was added portionwise over 30 mins maintaining a batch temperature <40° C. An exotherm of 25-30° C. was observed over the addition with cooling provided by a reduced jacket temperature of 15° C. The reaction was stirred for 4 h at 20-30° C. after which HPLC indicated 93.2% of desired material. The batch was split for workup with each half of the mixture diluted with DCM (28.6 L) and washed with water (3×7.8 L). The organics were dried over MgSO₄ (˜550 g) and filtered, washing with DCM (4 L). The combined organics were then concentrated to give 8444 g of the desired material as an oil (8434 g, 106% yield, 94.7% purity by HPLC). Analytical data was consistent with that obtained from previous batches.

### Intermediate B8: 5-Bromo-2-fluorobenzoyl Chloride

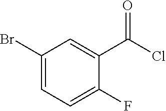 [SMILES: O=C(Cl)c1cc(Br)ccc1F]

Thionyl chloride (75.0 mL, 1027.36 mmol) was added drop-wise to 5-bromo-2-fluorobenzoic acid (150 g, 684.91 mmol), in toluene (1.2 L) and DMF (12 mL) at ambient temperature over a period of 1 h. The resulting mixture was stirred at 70° C. for 16 h then the mixture allowed to cool and concentrated in vacuo to afford the desired material (160 g, 98%) as light yellow oil, which was used without further purification. NMR Spectrum: ¹H NMR (400 MHz, DMSO-d6) δ 7.26-7.31 (1H, m), 7.83 (1H, dd), 8.02 (1H, d).

On a larger scale, 3-bromo-6-fluorobenzoic acid (3888 g, 17.75 mol) was charged to the vessel at 20° C. followed by toluene (29.2 L). Thionyl chloride (1950 ml, 26.88 mol) was added, followed by DMF (310 mL) with no observed exotherm. The mixture was heated to 65-75° C. (solution obtained above ˜45° C.) with no observed exotherm and slight gas evolution. The reaction was stirred for 40 h at this temperature at which point HPLC analysis showed 87.6% product, 3.4% starting material. The reaction was concentrated in vacuo and azeotroped with toluene (18 L) to give 4328 g of the desired material (103% yield, 87.3% by HPLC).

### Example 5

### 8-[4-[2-(Dimethylamino)ethoxy]phenyl]-1-isopropyl-3-methyl-imidazo[4,5-c]quinolin-2-one

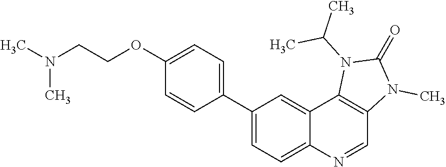

(4-(2-(Dimethylamino)ethoxy)phenyl)boronic acid (62.7 mg, 0.30 mmol), 8-bromo-1-isopropyl-3-methyl-imidazo[4,5-c]quinolin-2-one (80 mg, 0.25 mmol) and 2M K₂CO₃ solution (0.375 mL, 0.75 mmol) were dissolved in dioxane (1.8 mL) and the mixture degassed. Dichloro[1,1′-bis(di-tert-butylphosphino)ferrocene]palladium(II) (8.14 mg, 0.01 mmol) was added and the reaction heated to 80° C. for 30 minutes in a sealed vessel using a microwave reactor. The reaction mixture was allowed to cool to ambient temperature then diluted with EtOAc (50 mL), washed with water (2×10 mL), saturated brine (20 mL) and the organic layer dried with a phase separating cartridge and evaporated to afford crude product. The crude product was purified by FCC, elution gradient 0 to 10% MeOH in DCM. The desired material was further purified by passage through a PL-Thiol (metal scavenging) resin cartridge, eluting with MeOH, to afford the desired material as a beige dry film (35.0 mg, 34.6%). NMR Spectrum: ¹H NMR (500 MHz, CDCl₃) δ 1.79 (6H, s), 2.48 (6H, s), 2.93 (2H, s), 3.59 (3H, s), 4.24 (2H, s), 5.31 (1H, d), 7.06-7.11 (2H, m), 7.6-7.66 (2H, m), 7.82 (1H, dd), 8.19 (1H, d), 8.32 (1H, s), 8.68 (1H, s). Mass Spectrum: m/z (ES+)[M+H]+=405

The material could also be isolated as a methanesulfonic acid salt using the following procedure:

The isolated material (35 mg, 0.09 mmol) was dissolved in DCM (2 mL) and 1M methanesulfonic acid in DCM (0.092 mL, 0.09 mmol) was added. The solution was evaporated to dryness and dried in a vacuum oven for 4 h to afford the desired material as a methaesulfonic acid salt. NMR Spectrum: ¹H NMR (500 MHz, DMSO-d6) δ 1.70 (6H, d), 2.31 (3H, s), 2.91 (6H, s), 3.52 (3H, s), 3.58 (3H, s), 4.39-4.45 (2H, m), 5.16-5.49 (1H, m), 7.18-7.24 (2H, m), 7.82-7.87 (2H, m), 7.97 (1H, d), 8.16 (1H, d), 8.39 (1H, s), 8.94 (1H, s), 9.59 (1H, s). Mass Spectrum: m/z (ES+)[M+H]+=405

(4-(2-(Dimethylamino)ethoxy)phenyl)boronic acid is commercially available and the preparation of 8-bromo-1-isopropyl-3-methyl-imidazo[4,5-c]quinolin-2-one has been described previously.

### Example 6

### 8-[4-[3-(Dimethylamino)propoxy]phenyl]-1-[(1S,3S)-3-methoxycyclopentyl]-3-methyl-imidazo[4,5-c]quinolin-2-one

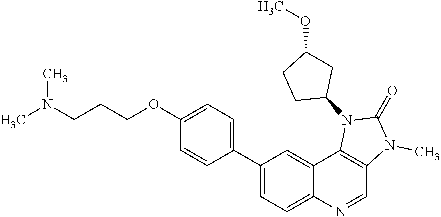

Dichlorobis(di-tert-butyl(3-sulfopropyl)phosphonio)palladate(II) (0.05M in water) (0.532 mL, 0.03 mmol) was added to a degassed mixture of N,N-dimethyl-3-[4-(4,4,5,5-tetramethyl-1,3,2-dioxaborolan-2-yl)phenoxy]propan-1-amine (162 mg, 0.53 mmol), 8-bromo-1-[(1S,3S)-3-methoxycyclopentyl]-3-methyl-imidazo[4,5-c]quinolin-2-one (200 mg, 0.53 mmol) and 2M K₂CO₃ solution (0.797 mL, 1.59 mmol) in 1,4-dioxane (1.772 mL) and water (0.443 mL) and the reaction heated to 80° C. for 4 h. The reaction mixture was evaporated to dryness, re-dissolved in DCM (50 mL), washed with water (50 mL) and the organic layer dried with a phase separating cartridge, filtered and evaporated to afford crude product. The crude product was purified by FCC, elution gradient 0 to 10% MeOH in DCM followed by 2M NH₃ in MeOH (10%) in DCM, to afford the desired material as a yellow solid (133 mg, 52.7%). NMR Spectrum: ¹H NMR (500 MHz, CDCl₃) δ 1.89-1.98 (1H, m), 1.97-2.05 (2H, m), 2.28 (6H, s), 2.30 (2H, s), 2.44-2.52 (2H, m), 2.52-2.64 (1H, m), 2.73 (1H, ddd), 3.37 (3H, s), 3.49 (1H, s), 3.58 (3H, s), 4.10 (2H, t), 4.17 (1H, dt), 5.62 (1H, p), 7.02-7.08 (2H, m), 7.61-7.67 (2H, m), 7.84 (1H, dd), 8.18 (1H, d), 8.33 (1H, d), 8.67 (1H, s). Mass Spectrum: m/z (ES+)[M+H]+=475.

The optical rotation of the sample was measured as −37° (measurement taken at 589 nm at 22.5° C. with a sample concentration approximately 2 mg/mL in EtOH)

This material can also be isolated as the methanesulfonic acid salt by dissolving in a small quantity of water and treating with an equivalent of methanesulfonic acid dissolved in a small quantity of water and then removing the water by lyophilisation.

NMR Spectrum: ¹H NMR (300 MHz, MeOH-d4) δ 1.90-2.03 (1H, m), 2.19-2.39 (5H, m), 2.45-2.71 (2H, m), 2.71 (3H, s), 2.95 (6H, s), 3.37 (3H, s), 3.31-3.43 (2H, m), 3.57 (3H, s), 4.11-4.26 (3H, m), 5.55-5.73 (1H, m), 7.12 (2H, d), 7.71 (2H, d), 7.90 (1H, dd), 8.10 (1H, d), 8.37 (1H, d), 8.75 (1H, s). Mass Spectrum: m/z (ES+)[M+H]+=475.

The preparation of 8-bromo-1-[(1S,3S)-3-methoxycyclopentyl]-3-methyl-imidazo[4,5-c]quinolin-2-one is described below:

### Intermediate C1: 8-Bromo-1-[(1S,3S)-3-methoxycyclopentyl]-3-methyl-imidazo[4,5-c]quinolin-2-one

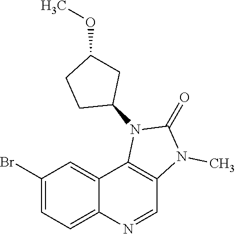 [SMILES: CO[C@H]1CC[C@H](n2c(=O)n(C)c3cnc4ccc(Br)cc4c32)C1]

NaH (60% in mineral oil) (0.444 g, 11.11 mmol) was added to a mixture of 8-bromo-1-[(1S,3S)-3-methoxycyclopentyl]-3H-imidazo[4,5-c]quinolin-2-one (1.15 g, 3.17 mmol) in DMF (15 mL) under nitrogen at 0° C. then the mixture stirred for 30 minutes. Methyl iodide (0.596 mL, 9.52 mmol) was added and the reaction mixture was stirred at ambient temperature for 16 h. Water was slowly added to the reaction and the solid filtered under vacuum and dried in a vacuum oven for 3 h to afford the desired material as a white solid (674 mg—slightly contaminated with residual DMF). NMR Spectrum: ¹H NMR (500 MHz, DMSO-d6) δ 1.22 (1H, s), 1.74-1.92 (1H, m), 2.11-2.24 (3H, m), 2.25-2.33 (1H, m), 3.27 (3H, s), 3.49 (3H, s), 4.07-4.15 (1H, m), 5.27-5.53 (1H, m), 7.74 (1H, dd), 7.98 (1H, dd), 8.36 (1H, s), 8.91 (1H, s). Mass Spectrum: m/z (ES+)[M+H]+=376.

### Intermediate C2: 8-Bromo-1-[(1S,3S)-3-methoxycyclopentyl]-3H-imidazo[4,5-c]quinolin-2-one

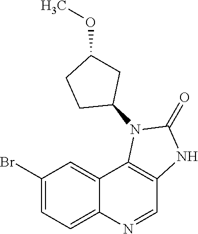 [SMILES: CO[C@H]1CC[C@H](n2c(=O)[nH]c3cnc4ccc(Br)cc4c32)C1]

Diphenyl phosphoryl azide (1.075 ml, 4.99 mmol) was added to a mixture of 6-bromo-4-[[(1S,3S)-3-methoxycyclopentyl]amino]quinoline-3-carboxylic acid (1.46 g, 4.16 mmol) and triethylamine (1.738 mL, 12.47 mmol) in DMF (9 mL) under nitrogen and the reaction heated at 60° C. for 4 h. The reaction was cooled to ambient temperature, the solid filtered under vacuum and washed with water. The solid was dried in a vacuum oven overnight to afford the desired material. An additional crop of material was isolated by repeating the filtration step and combined with the previous crop (1.15 g, 79%). NMR Spectrum: ¹H NMR (500 MHz, DMSO-d6) δ 1.56-1.82 (1H, m), 1.98 (1H, t), 2.08-2.31 (3H, m), 2.46 (1H, s), 4.43 (1H, s), 4.78 (1H, d), 5.26-5.64 (1H, m), 7.73 (1H, dd), 7.96 (1H, dd), 8.35 (1H, s), 8.67 (1H, s), 11.62 (1H, s). Mass Spectrum: m/z (ES+)[M+H]+=348.

### Intermediate C3: 6-Bromo-4-[[(1S,3S)-3-methoxycyclopentyl]amino]quinoline-3-carboxylic acid

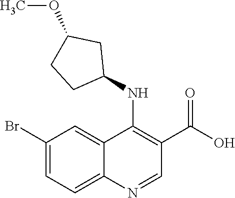 [SMILES: CO[C@H]1CC[C@H](Nc2c(C(=O)O)cnc3ccc(Br)cc23)C1]

NaOH (2M) (13.98 mL, 27.95 mmol) was added to a mixture of ethyl 6-bromo-4-[[(1S,3S)-3-methoxycyclopentyl]amino]quinoline-3-carboxylate (2.65 g, 6.99 mmol) in THF (15 mL) and the reaction heated at 60° C. for 5 h. The reaction was cooled to ambient temperature and the organic solvent removed under reduced pressure. The aqueous residue was adjusted to pH7 using hydrochloric acid (2M) and the solid was filtered under vacuum and dried in a vacuum oven for 24 h to afford, the desired material as a grey solid (1.46 g).

Mass Spectrum: m/z (ES+)[M+H]+=351.

### Intermediate C4: Ethyl 6-bromo-4-[[(1S,3S)-3-methoxycyclopentyl]amino]quinoline-3-carboxylate

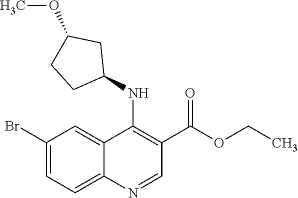 [SMILES: CCOC(=O)c1cnc2ccc(Br)cc2c1N[C@H]1CC[C@H](OC)C1]

Triethylamine (3.90 mL, 27.98 mmol) was added to (1S,3S)-3-aminocyclopentanol hydrochloride salt (1 g, 7.27 mmol) in acetonitrile (15.6 mL) and stirred for 5 minutes. ethyl 6-bromo-4-chloroquinoline-3-carboxylate (2.2 g, 6.99 mmol) was added and the reaction mixture was heated at 100° C. for 2 h. The solid was isolated by filtration, dissolved in DCM and washed with water. The filtrate was concentrated to dryness and the residue dissolved in DCM (25 mL) and washed with water (25 mL). The organics were combined and dried over a phase separating cartridge and the solvent was removed under reduced pressure to afford the desired material as an orange solid (2.65 g) and used directly without further purification. Mass Spectrum: m/z (ES+)[M+H]+=379.

The preparation of ethyl 6-bromo-4-chloroquinoline-3-carboxylate has been described previously.

### Example 7

### 8-[4-[3-(Dimethylamino)propoxy]phenyl]-1-[(1R,3R)-3-methoxycyclopentyl]-3-methyl-imidazo[4,5-c]quinolin-2-one

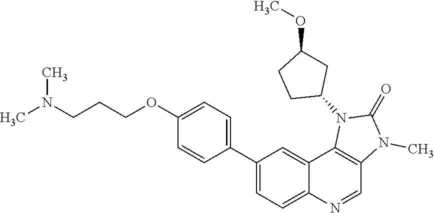

Pd(Ph₃P)₄ (0.369 g, 0.32 mmol) was added to N,N-dimethyl-3-[4-(4,4,5,5-tetramethyl-1,3,2-dioxaborolan-2-yl)phenoxy]propan-1-amine (0.973 g, 3.19 mmol), 8-bromo-1-[(1R,3R)-3-methoxycyclopentyl]-3-methylimidazo[4,5-c]quinolin-2-one: 8-bromo-1-[(1S,3S)-3-methoxycyclopentyl]-3-methylimidazo[4,5-c]quinolin-2-one (1:1 mixture) (1.2 g, 3.19 mmol) and Na₂CO₃ (0.676 g, 6.38 mmol) in 1,4-dioxane (30 mL) and water (6 mL) under nitrogen and the resulting mixture stirred at 80° C. for 16 h. The solvent was removed under reduced pressure and the crude product was purified by flash C18-flash chromatography, elution gradient 0 to 80% MeOH in water, to yield the desired material as a racemic mixture and a yellow solid (0.80 g, 52.9%).

The racemic mixture was separated by preparative chiral-HPLC on a AD column, eluting isocratically with 85% hexane in IPA (modified with diethylamine) as eluent, to afford the first eluting product as solid (330 mg, 47.1%), and the second eluting product as a pale yellow solid (290 mg, 41.4%). The isolated enantiomers were converted to the corresponding methanesulfonic acid salt by dissolving the material in a small quantity of water and treating with one equivalent of methanesulfonic acid in water and then removing the water by lyophilisation. Optical rotation was used to identify the chirality by comparison with the chirally prepared sample of 8-[4-[3-(dimethylamino)propoxy]phenyl]-1-[(1S,3S)-3-methoxycyclopentyl]-3-methyl-imidazo[4,5-c]quinolin-2-one (Example 6).

Isomer 1, 8-[4-[3-(dimethylamino)propoxy]phenyl]-1-[(1R,3R)-3-methoxycyclopentyl]-3-methyl-imidazo[4,5-c]quinolin-2-one (Example 7)—(352 mg, optical rotation+32°): (Free base) NMR Spectrum: ¹H NMR (300 MHz, MeOH-d4) δ 1.91-2.12 (3H, m), 2.21-2.56 (4H, m), 2.43 (6H, s), 2.63 (3H, dd), 3.37 (3H, d), 3.50-3.59 (3H, m), 4.05-4.19 (3H, m), 5.55-5.65 (1H, m), 7.06 (2H, dd), 7.66 (2H, d), 7.88 (1H, d), 8.07 (1H, d), 8.32 (1H, d), 8.70 (1H, s). (Methane sulfonic acid salt) NMR Spectrum: ¹H NMR (300 MHz, MeOH-d4) δ 1.90-2.02 (1H, m), 2.18-2.40 (5H, m), 2.44-2.56 (1H, m), 2.56-2.67 (1H, m), 2.71 (3H, s), 2.99 (6H, s), 3.34-3.48 (5H, m), 3.57 (3H, s), 4.11-4.26 (3H, m), 5.54-5.71 (1H, m), 7.12 (2H, d), 7.70 (2H, d), 7.93 (1H, dd), 8.10 (1H, d), 8.37 (1H, d), 8.77 (1H, s). Mass Spectrum: m/z (ES+)[M+H]+=475.

Isomer 2, 8-[4-[3-(dimethylamino)propoxy]phenyl]-1-[(1S,3S)-3-methoxycyclopentyl]-3-methyl-imidazo[4,5-c]quinolin-2-one (Example 6)—(322 mg, optical rotation −34.8°): (Free base) NMR Spectrum: ¹H NMR (300 MHz, MeOH-d4) δ 1.88-2.01 (1H, m), 2.09-2.37 (5H, m), 2.43-2.67 (2H, m), 2.69 (6H, s), 2.97-3.11 (2H, m), 3.37 (3H, s), 3.54 (3H, s), 4.15 (3H, t), 5.50-5.68 (1H, m), 7.08 (2H, d), 7.67 (2H, d), 7.86 (1H, dd), 8.05 (1H, d), 8.30 (1H, d), 8.56 (1H, s), 8.70 (1H, s). (Methane sulfonic acid salt) NMR Spectrum: ¹H NMR (300 MHz, MeOH-d4) δ 1.90-2.03 (1H, m), 2.19-2.39 (5H, m), 2.45-2.71 (2H, m), 2.71 (3H, s), 2.95 (6H, s), 3.37 (3H, s), 3.31-3.43 (2H, m), 3.57 (3H, s), 4.11-4.26 (3H, m), 5.55-5.73 (1H, m), 7.12 (2H, d), 7.71 (2H, d), 7.90 (1H, dd), 8.10 (1H, d), 8.37 (1H, d), 8.75 (1H, s). Mass Spectrum: m/z (ES+)[M+H]+=475.

The following compounds were prepared in an analogous fashion from the appropriate boronic acid and bromo intermediate, purified by appropriate chromatographic techniques and isolated as either the free base or methanesulfonic acid salt.

| Example | Structure | Name |
|---|---|---|
| 8* | 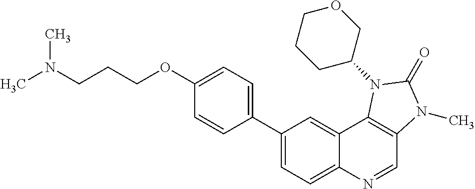 | 8-[4-[3- (dimethylamino)propoxy]phenyl]-3- methyl-1-[(3R)-tetrahydropyran-3- yl]imidazo[4,5-c]quinolin-2-one |
| 9* | 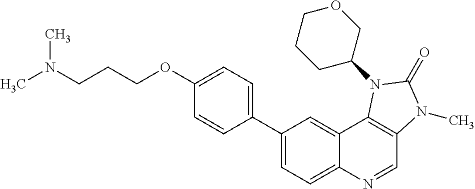 | 8-[4-[3- (dimethylamino)propoxy]phenyl]-3- methyl-1-[(3S)-tetrahydropyran-3- yl]imidazo[4,5-c]quinolin-2-one |
| 10** | 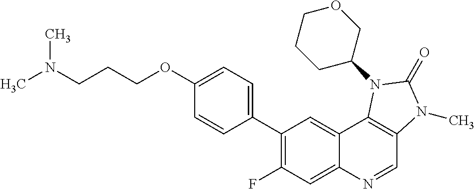 | 8-[4-[3- (dimethylamino)propoxy]phenyl]-7- fluoro-3-methyl-1-[(3S)- tetrahydropyran-3-yl]imidazo[4,5- c]quinolin-2-one |
| 11*** | 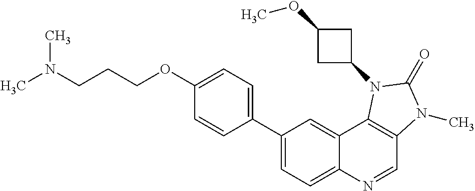 | 8-[4-[3- (dimethylamino)propoxy]phenyl]-1- (cis-3-methoxycyclobutyl)-3-methyl- imidazo[4,5-c]quinolin-2-one |
| 12**** | 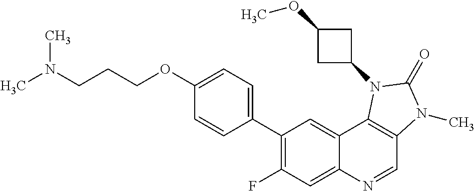 | 8-[4-[3- (dimethylamino)propoxy]phenyl]-7- fluoro-1-(cis3-methoxycyclobutyl)-3- methyl-imidazo[4,5-c]quinolin-2-one |
| 13*** | 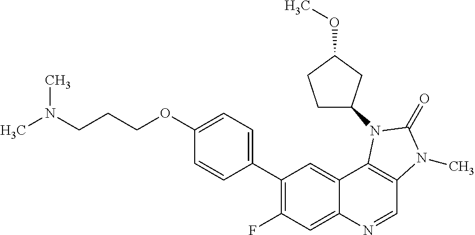 | 8-[4-[3- (dimethylamino)propoxy]phenyl]-7- fluoro-1-[trans-3- methoxycyclopentyl]-3-methyl- imidazo[4,5-c]quinolin-2-one—isomer 2 |
| 14*** | 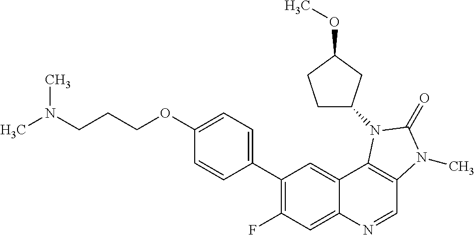 | 8-[4-[3- (dimethylamino)propoxy]phenyl]-7- fluoro-1-[trans-3- methoxycyclopentyl]-3-methyl- imidazo[4,5-c]quinolin-2-one—isomer 1 |
| 15** | 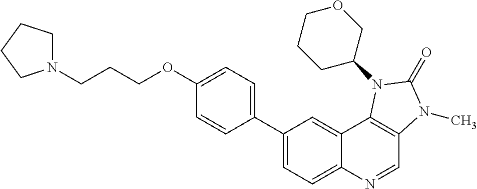 [SMILES: Cn1c(=O)n([C@H]2CCCOC2)c2c3cc(-c4ccc(OCCCN5CCCC5)cc4)ccc3ncc21] | 3-methyl-8-[4-(3-pyrrolidin-1- ylpropoxy)phenyl]-1-[(3S)- tetrahydropyran-3-yl]imidazo[4,5- c]quinolin-2-one |
| 16***** | 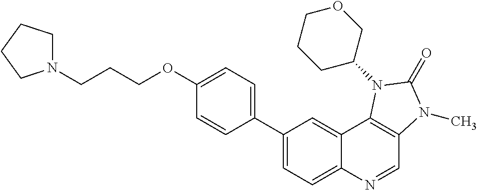 [SMILES: Cn1c(=O)n([C@@H]2CCCOC2)c2c3cc(-c4ccc(OCCCN5CCCC5)cc4)ccc3ncc21] | 3-methyl-8-[4-(3-pyrrolidin-1- ylpropoxy)phenyl]-1-[(3R)- tetrahydropyran-3-yl]imidazo[4,5- c]quinolin-2-one |
| 17** | 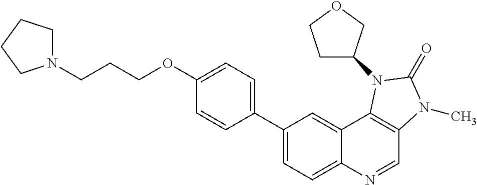 [SMILES: Cn1c(=O)n([C@H]2CCOC2)c2c3cc(-c4ccc(OCCCN5CCCC5)cc4)ccc3ncc21] | 3-methyl-8-[4-[3-(1- piperidyl)propoxy]phenyl]-1-[(3S)- tetrahydrofuran-3-yl]imidazo[4,5- c]quinolin-2-one |
| 18** | 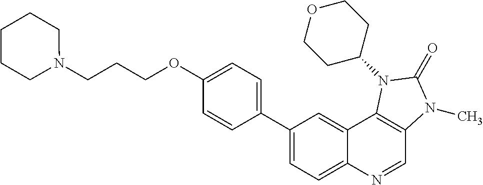 | 3-methyl-8-[4-[3-(1- piperidyl)propoxy]phenyl]-1- tetrahydropyran-4-yl-imidazo[4,5- c]quinolin-2-one |
| 19*** | 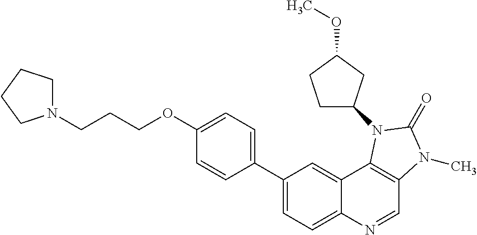 [SMILES: CO[C@H]1CC[C@H](n2c(=O)n(C)c3cnc4ccc(-c5ccc(OCCCN6CCCC6)cc5)cc4c32)C1] | 1-[trans-3-methoxycyclopentyl]-3- methyl-8-[4-(3-pyrrolidin-1- ylpropoxy)phenyl]imidazo[4,5- c]quinolin-2-one—Isomer 1 |
| 20*** | 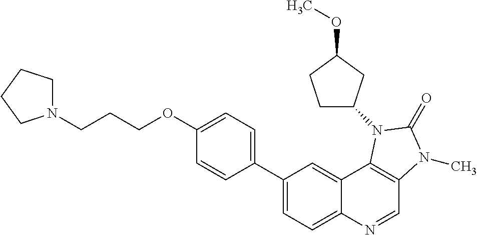 [SMILES: CO[C@@H]1CC[C@@H](n2c(=O)n(C)c3cnc4ccc(-c5ccc(OCCCN6CCCC6)cc5)cc4c32)C1] | 1-[trans-3-methoxycyclopentyl]-3- methyl-8-[4-(3-pyrrolidin-1- ylpropoxy)phenyl]imidazo[4,5- c]quinolin-2-one—Isomer 2 |
| 21** | 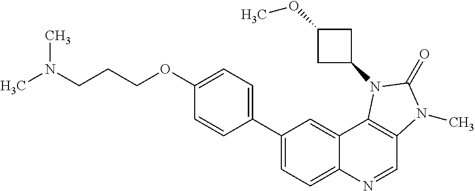 | 8-[4-[3- (dimethylamino)propoxy]phenyl]-1- (trans-3-methoxycyclobutyl)-3-methyl- imidazo[4,5-c]quinolin-2-one |
| 22**** | 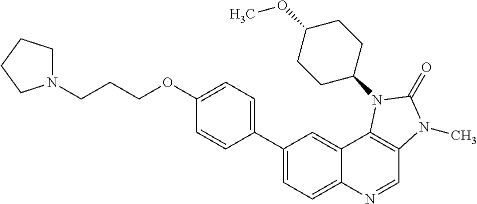 [SMILES: CO[C@H]1CC[C@H](n2c(=O)n(C)c3cnc4ccc(-c5ccc(OCCCN6CCCC6)cc5)cc4c32)CC1] | 1-(trans-4-methoxycyclohexyl)-3- methyl-8-[4-(3-pyrrolidin-1- ylpropoxy)phenyl]imidazo[4,5- c]quinolin-2-one |
| 23*** | 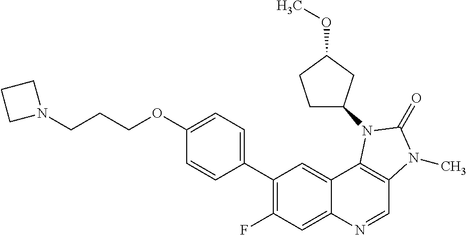 [SMILES: CO[C@H]1CC[C@H](n2c(=O)n(C)c3cnc4cc(F)c(-c5ccc(OCCCN6CCC6)cc5)cc4c32)C1] | 8-[4-[3-(azetidin-1- yl)propoxy]phenyl]-7-fluoro-1-[trans- 3-methoxycyclopentyl]-3-methyl- imidazo[4,5-c]quinolin-2-one—Isomer 2 |
| 24*** | 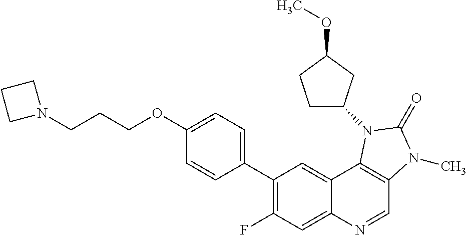 [SMILES: CO[C@@H]1CC[C@@H](n2c(=O)n(C)c3cnc4cc(F)c(-c5ccc(OCCCN6CCC6)cc5)cc4c32)C1] | 8-[4-[3-(azetidin-1- yl)propoxy]phenyl]-7-fluoro-1-[trans- 3-methoxycyclopentyl]-3-methyl- imidazo[4,5-c]quinolin-2-one—Isomer 1 |
| 25**** | 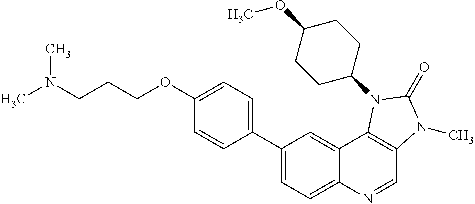 | 8-[4-[3- (dimethylamino)propoxy]phenyl]-1- (cis-4-methoxycyclohexyl)-3-methyl- imidazo[4,5-c]quinolin-2-one |
| 26**** | 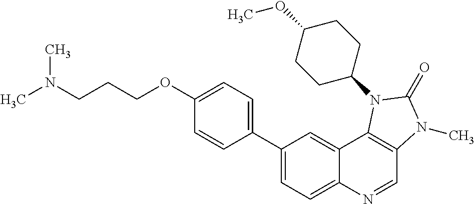 | 8-[4-[3- (dimethylamino)propoxy]phenyl]-1- (trans-4-methoxycyclohexyl)-3- methyl-imidazo[4,5-c]quinolin-2-one |
| 27**** | 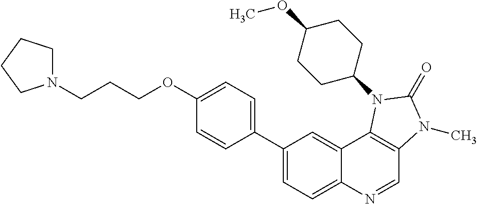 [SMILES: CO[C@H]1CC[C@@H](n2c(=O)n(C)c3cnc4ccc(-c5ccc(OCCCN6CCCC6)cc5)cc4c32)CC1] | 1-(cis-4-methoxycyclohexyl)-3- methyl-8-[4-(3-pyrrolidin-1- ylpropoxy)phenyl]imidazo[4,5- c]quinolin-2-one |
| 28**** | 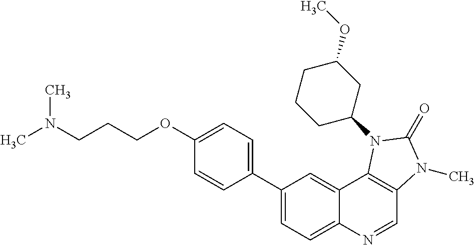 | 8-[4-[3- (dimethylamino)propoxy]phenyl]-1- [trans-3-methoxycyclohexyl]-3- methyl-imidazo[4,5-c]quinolin-2- one—Isomer 2 |
| 29**** | 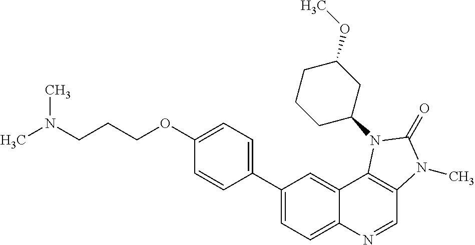 | 8-[4-[3- (dimethylamino)propoxy]phenyl]-1- [trans-3-methoxycyclohexyl]-3- methyl-imidazo[4,5-c]quinolin-2- one—Isomer 1 |
| 30**** | 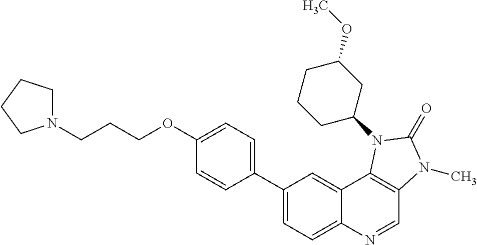 [SMILES: CO[C@H]1CCC[C@H](n2c(=O)n(C)c3cnc4ccc(-c5ccc(OCCCN6CCCC6)cc5)cc4c32)C1] | 1-[trans-3-methoxycyclohexyl]-3- methyl-8-[4-(3-pyrrolidin-1- ylpropoxy)phenyl]imidazo[4,5- c]quinolin-2-one—Isomer 2 |
| 31**** | 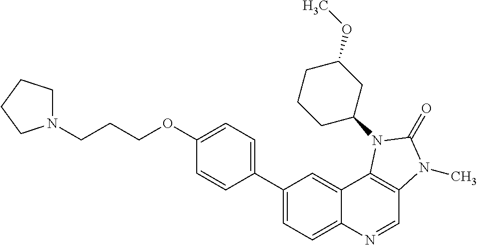 [SMILES: CO[C@H]1CCC[C@H](n2c(=O)n(C)c3cnc4ccc(-c5ccc(OCCCN6CCCC6)cc5)cc4c32)C1] | 1-[trans-3-methoxycyclohexyl]-3- methyl-8-[4-(3-pyrrolidin-1- ylpropoxy)phenyl]imidazo[4,5- c]quinolin-2-one—Isomer 1 |
| 32**** | 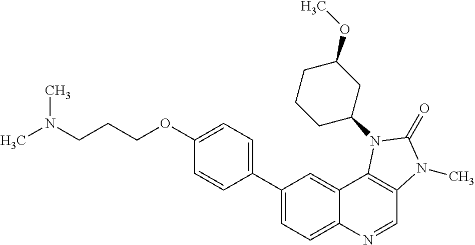 | 8-[4-[3- (dimethylamino)propoxy]phenyl]-1- [cis-3-methoxycyclohexyl]-3-methyl- imidazo[4,5-c]quinolin-2-one (prepared from Intermediate T1). |
| 33**** | 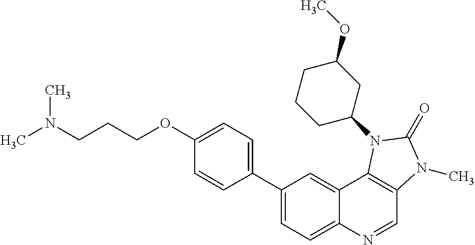 | 8-[4-[3- (dimethylamino)propoxy]phenyl]-1- [cis-3-methoxycyclohexyl]-3-methyl- imidazo[4,5-c]quinolin-2-one (prepared from Intermediate S1). |
| 34*** | 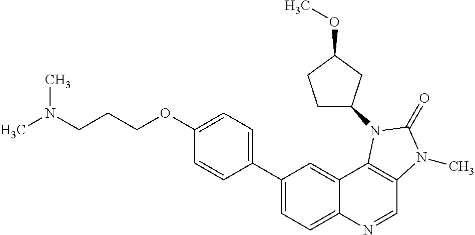 | 8-[4-[3- (dimethylamino)propoxy]phenyl]-1- [cis-3-methoxycyclopentyl]-3-methyl- imidazo[4,5-c]quinolin-2-one—Isomer 2 |
| 35*** | 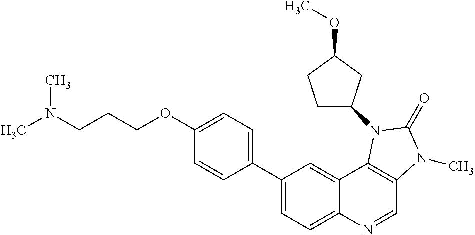 | 8-[4-[3- (dimethylamino)propoxy]phenyl]-1- [cis-3-methoxycyclopentyl]-3-methyl- imidazo[4,5-c]quinolin-2-one—Isomer 1 |
| 36**** | 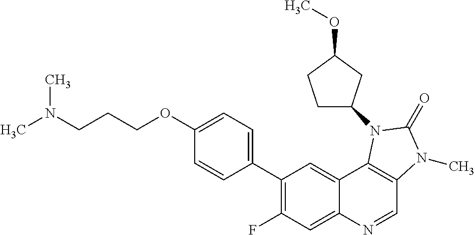 | 8-[4-[3- (dimethylamino)propoxy]phenyl]-7- fluoro-1-[cis-3-methoxycyclopentyl]- 3-methyl-imidazo[4,5-c]quinolin-2- one—Isomer 1 |
| 37**** | 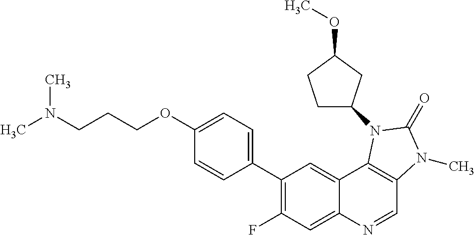 | 8-[4-[3- (dimethylamino)propoxy]phenyl]-7- fluoro-1-[cis-3-methoxycyclopentyl]- 3-methyl-imidazo[4,5-c]quinolin-2- one—Isomer 2 |
| 38**** | 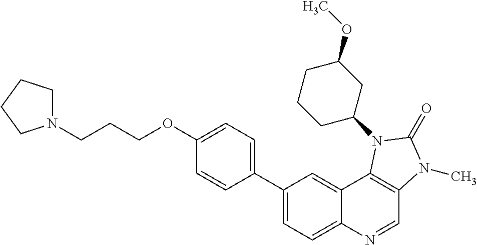 [SMILES: CO[C@@H]1CCC[C@H](n2c(=O)n(C)c3cnc4ccc(-c5ccc(OCCCN6CCCC6)cc5)cc4c32)C1] | 1-[cis-3-methoxycyclohexyl]-3- methyl-8-[4-(3-pyrrolidin-1- ylpropoxy)phenyl]imidazo[4,5- c]quinolin-2-one (prepared from Intermediate T1). |
| 39**** | 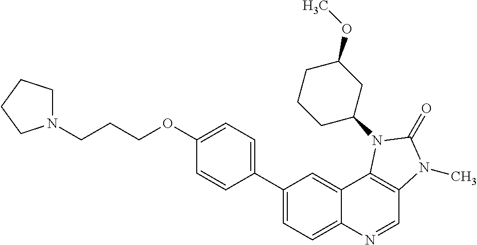 [SMILES: CO[C@@H]1CCC[C@H](n2c(=O)n(C)c3cnc4ccc(-c5ccc(OCCCN6CCCC6)cc5)cc4c32)C1] | 1-[cis-3-methoxycyclohexyl]-3- methyl-8-[4-(3-pyrrolidin-1- ylpropoxy)phenyl]imidazo[4,5- c]quinolin-2-one (prepared from Intermediate S1). |
| 40** | 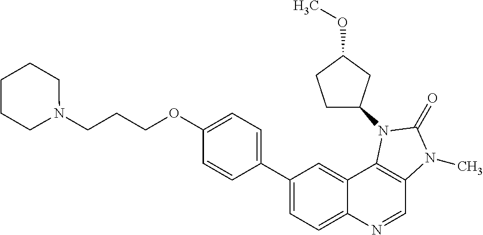 [SMILES: CO[C@H]1CC[C@H](n2c(=O)n(C)c3cnc4ccc(-c5ccc(OCCCN6CCCCC6)cc5)cc4c32)C1] | 1-[(1S,3S)-3-methoxycyclopentyl]-3- methyl-8-[4-[3-(1- piperidyl)propoxy]phenyl]imidazo[4,5- c]quinolin-2-one |
| 41**** | 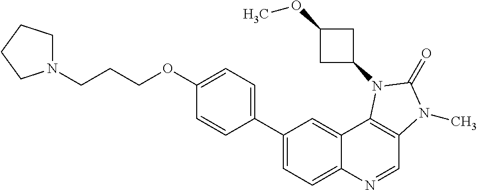 [SMILES: CO[C@H]1C[C@@H](n2c(=O)n(C)c3cnc4ccc(-c5ccc(OCCCN6CCCC6)cc5)cc4c32)C1] | 1-(cis-3-methoxycyclobutyl)-3-methyl- 8-[4-(3-pyrrolidin-1- ylpropoxy)phenyl]imidazo[4,5- c]quinolin-2-one |
| 42** | 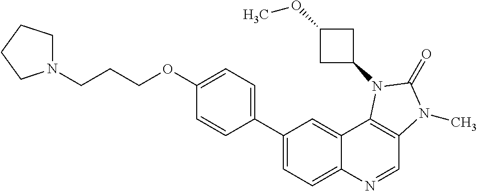 [SMILES: CO[C@H]1C[C@H](n2c(=O)n(C)c3cnc4ccc(-c5ccc(OCCCN6CCCC6)cc5)cc4c32)C1] | 1-(trans-3-methoxycyclobutyl)-3- methyl-8-[4-(3-pyrrolidin-1- ylpropoxy)phenyl]imidazo[4,5- c]quinolin-2-one |
| 43** | 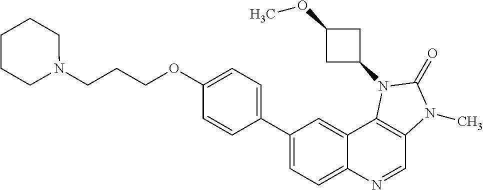 [SMILES: CO[C@H]1C[C@@H](n2c(=O)n(C)c3cnc4ccc(-c5ccc(OCCCN6CCCCC6)cc5)cc4c32)C1] | 1-(cis-3-methoxycyclobutyl)-3-methyl- 8-[4-[3-(1- piperidyl)propoxy]phenyl]imidazo[4,5- c]quinolin-2-one |
| 44** | 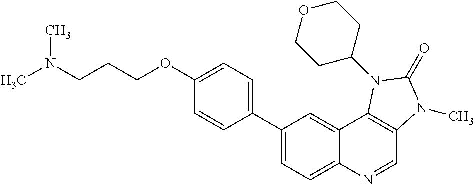 | 8-[4-[3- (dimethylamino)propoxy]phenyl]-3- methyl-1-tetrahydropyran-4-yl- imidazo[4,5-c]quinolin-2-one |

*The reaction used dichloro[1,1′-bis(di-tert-butylphosphino)ferrocene]palladium(II) as the catalyst with K₂CO₃ as the base and was heated at 90° C. for 30 mins.
**The reaction used dichlorobis(di-tert-butyl(3-sulfopropyl)phosphonio)palladate(II) (0.05M in water) as the catalyst with K₂CO₃ as the base and was heated at 80° C. for between 1 to 6 h.
***The reaction used chloro(2-dicyclohexylphosphino-2′,4′,6′-triisopropyl-1,1′-biphenyl)[2-(2′-amino-1,1′-biphenyl)]palladium(II) as the catalyst with Cs₂CO₃ as the base and was heated at 80° C. for 4 h.
****The reaction used tetrakis(triphenylphosphine)palladium(0) as the catalyst with either Na₂CO₃ or Cs₂CO₃ as the base and was heated at 80-90° C. for between 2-16 h.
*****The reaction used [1,1′-bis(di-tert-butylphosphino)ferrocene]dichloropalladium(II) as the catalyst with K₃PO₄ as the base and was heated at 80° C. for 4 h.

Examples 13 & 14 were separated from a racemic mixture by preparative chiral-HPLC, eluting isocratically with 85% hexane in IPA (modified with diethylamine) as eluent, to afford Example 14 as the first eluting product and Example 13 as the second eluting product.

Examples 19 & 20 were separated from a racemic mixture by preparative chiral-HPLC, eluting isocratically with 85% hexane in IPA (modified with diethylamine) as eluent, to afford Example 20 as the first eluting product and Example 19 as the second eluting product.

Examples 23 & 24 were separated from a racemic mixture by preparative chiral-HPLC, eluting isocratically with 85% hexane in IPA (modified with diethylamine) as eluent, to afford Example 24 as the first eluting product and Example 23 as the second eluting product.

Examples 28 & 29 were separated from a racemic mixture by preparative chiral-HPLC, eluting isocratically with 80% hexane in IPA (modified with diethylamine) as eluent, to afford Example 29 as the first eluting product and Example 28 as the second eluting product.

Examples 30 & 31 were separated from a racemic mixture by preparative chiral-HPLC, eluting isocratically with 80% hexane in EtOH (modified with diethylamine) as eluent, to afford Example 31 as the first eluting product and Example 30 as the second eluting product.

Examples 34 & 35 were separated from a racemic mixture by preparative chiral-HPLC, eluting isocratically with 70% hexane in EtOH (modified with diethylamine) as eluent, to afford Example 35 as the first eluting product and Example 34 as the second eluting product.

Examples 36 & 37 were separated from a racemic mixture by preparative chiral-HPLC, eluting isocratically with 80% hexane in IPA (modified with diethylamine) as eluent, to afford Example 36 as the first eluting product and Example 37 as the second eluting product.

### Example 8

(Free base) NMR Spectrum: ¹H NMR (500 MHz, CDCl₃) δ 1.91 (2H, d), 2.08 (2H, d), 2.19-2.29 (1H, m), 2.40 (5H, s), 2.55-2.71 (2H, m), 2.71-2.89 (2H, m), 3.56 (3H, s), 3.57-3.61 (1H, m), 4.04 (1H, d), 4.12 (2H, t), 4.19 (1H, d), 4.54 (1H, t), 4.92-5.12 (1H, m), 7.06 (2H, d), 7.64 (2H, d), 7.85 (1H, dd), 8.19 (1H, d), 8.32 (1H, s), 8.66 (1H, s). (Methanesulfonic acid salt) NMR Spectrum: ¹H NMR (500 MHz, DMSO-d6) δ 1.74-1.97 (2H, m), 2.11-2.21 (2H, m), 2.32 (3H, s), 2.64-2.73 (1H, m), 2.86 (6H, d), 3.23-3.32 (2H, m), 3.39-3.47 (2H, m), 3.52 (3H, s), 3.95 (1H, d), 4.16 (3H, t), 4.26 (1H, t), 4.92-5.12 (1H, m), 7.17 (2H, d), 7.80 (2H, d), 8.05 (1H, s), 8.18 (1H, d), 8.37 (1H, s), 9.00 (1H, s), 9.37 (1H, s). Mass Spectrum: m/z (ES+)[M+H]+=461.

### Example 9

(Free base) NMR Spectrum: ¹H NMR (500 MHz, CDCl₃) δ 1.26 (2H, t), 1.91 (2H, d), 2.13-2.29 (3H, m), 2.52 (5H, s), 2.75-2.82 (2H, m), 3.56 (4H, s), 4.04 (1H, d), 4.13 (2H, t), 4.16-4.22 (1H, m), 4.54 (1H, t), 4.92-5.12 (1H, m), 7.02-7.08 (2H, m), 7.6-7.66 (2H, m), 7.84 (1H, dd), 8.19 (1H, d), 8.31 (1H, s), 8.66 (1H, s). (Methanesulfonic acid salt) NMR Spectrum: ¹H NMR (500 MHz, DMSO-d6) δ 1.71-1.98 (2H, m), 2.11-2.19 (2H, m), 2.32 (3H, s), 2.61-2.78 (2H, m), 2.86 (6H, d), 3.23-3.31 (2H, m), 3.39-3.49 (1H, m), 3.53 (3H, s), 3.95 (1H, d), 4.16 (3H, t), 4.26 (1H, t), 4.92-5.12 (1H, m), 7.15-7.21 (2H, m), 7.81 (2H, d), 8.11 (1H, s), 8.21 (1H, d), 8.40 (1H, s), 9.07 (1H, s), 9.32 (1H, s). Mass Spectrum: m/z (ES+), [M+H]+=461.

### Example 10

(Free base) NMR Spectrum: ¹H NMR (500 MHz, CDCl₃) δ 1.82-1.93 (2H, m), 1.99-2.06 (2H, m), 2.20 (1H, d), 2.30 (6H, s), 2.52 (2H, s), 2.69-2.87 (1H, m), 3.56 (3H, s), 4.01 (1H, d), 4.08-4.19 (3H, m), 4.52 (1H, t), 4.82-5.01 (1H, m), 7.03-7.1 (2H, m), 7.58 (2H, dd), 7.87 (1H, d), 8.20 (1H, d), 8.66 (1H, s). (Methanesulfonic acid salt) NMR Spectrum: ¹H NMR (500 MHz, DMSO-d6) δ 2.09-2.2 (3H, m), 2.32 (6H, s), 2.56-2.73 (1H, m), 2.84 (6H, d), 3.21-3.29 (2H, m), 3.31-3.46 (1H, m), 3.51 (3H, s), 3.89 (1H, d), 4.1-4.25 (3H, m), 4.87-5.03 (1H, m), 7.14-7.19 (2H, m), 7.69 (2H, dd), 8.01 (1H, d), 8.31 (1H, d), 9.14 (1H, s), 9.35 (1H, s). Mass Spectrum: m/z (ES+), [M+H]+=479.

### Example 11

(Free base) NMR Spectrum: ¹H NMR (300 MHz, CDCl₃) δ 2.18-2.35 (2H, m), 2.65 (6H, s), 2.92-3.06 (4H, m), 3.08-3.25 (2H, m), 3.31 (3H, s), 3.61 (3H, s), 3.84-4.00 (1H, m), 4.17 (2H, t), 4.85-4.96 (1H, m), 7.02 (2H, d), 7.63 (2H, d), 7.85 (1H, d), 8.17 (1H, d), 8.30 (1H, s), 8.68 (1H, s). Mass Spectrum: m/z (ES+), [M+H]+=461.

### Example 12

(Free base) NMR Spectrum: ¹H NMR (400 MHz, DMSO-d6) δ 1.8-1.9 (2H, m), 2.1-2.2 (6H, m), 2.3-2.4 (2H, m), 2.7-2.8 (2H, m), 2.9-3.0 (2H, m), 3.15 (3H, s), 3.48 (3H, s), 3.7-3.8 (1H, m), 4.0-4.1 (2H, m), 4.9-5.1 (1H, m), 7.0-9.0 (7H, m). Mass Spectrum: m/z (ES+), [M+H]+=479.

### Example 13

(Free base) NMR Spectrum: ¹H NMR (300 MHz, MeOH-d4) δ 1.71-1.83 (1H, m), 1.83-2.00 (2H, m), 2.00-2.25 (3H, m), 2.25 (6H, s), 2.25-2.37 (1H, m), 2.38-2.50 (1H, m), 2.52-2.61 (2H, m), 3.21 (3H, s), 3.45 (3H, s), 4.03 (3H, t), 5.36-5.42 (1H, m), 6.94 (2H, d), 7.43 (2H, d), 7.46 (1H, d), 8.07 (1H, d), 8.60 (1H, s). Mass Spectrum: m/z (ES+), [M+H]+=493.

### Example 14

(Free base) NMR Spectrum: ¹H NMR (300 MHz, MeOH-d4) δ 1.70-1.85 (1H, m), 1.85-2.09 (2H, m), 2.09-2.27 (3H, m), 2.27 (6H, s), 2.31-2.43 (1H, m), 2.43-2.69 (3H, m), 3.22 (3H, s), 3.44 (3H, s), 3.98-4.12 (3H, m), 5.39-5.44 (1H, m), 6.94 (2H, d), 7.45 (2H, d), 7.59 (1H, d), 8.10 (1H, d), 8.62 (1H, s). Mass Spectrum: m/z (ES+), [M+H]+=493.

### Example 15

(Free base) NMR Spectrum: ¹H NMR (500 MHz, CDCl₃) δ 1.81 (5H, td), 1.93 (2H, t), 1.99-2.11 (3H, m), 2.23 (1H, d), 2.56 (3H, s), 2.65 (2H, dt), 2.72-2.9 (1H, m), 3.58 (3H, s), 3.99-4.08 (1H, m), 4.12 (2H, t), 4.20 (1H, d), 4.54 (1H, t), 4.84-5.04 (1H, m), 7.03-7.1 (2H, m), 7.6-7.67 (2H, m), 7.85 (1H, dd), 8.19 (1H, d), 8.32 (1H, s), 8.66 (1H, s). (Methanesulfonic acid salt) NMR Spectrum: ¹H NMR (500 MHz, DMSO-d6) δ 1.76-1.85 (6H, m), 2.00 (1H, s), 2.16 (1H, d), 2.29 (1H, s), 2.53-2.99 (9H, m), 3.39 (1H, dd), 3.48 (3H, s), 3.93 (1H, d), 3.97-4.05 (1H, m), 4.11 (3H, t), 4.25 (1H, t), 4.84-5.04 (1H, m), 7.09-7.15 (2H, m), 7.71-7.77 (2H, m), 7.91 (1H, dd), 8.11 (1H, d), 8.31 (1H, s), 8.85 (1H, s), 9.41 (1H, s). Mass Spectrum: m/z (ES+), [M+H]+=487.

### Example 16

(Free base) NMR Spectrum: ¹H NMR (300 MHz, MeOH-d4) δ 1.89-1.96 (6H, m), 2.08-2.18 (2H, m), 2.23 (1H, d), 2.77-2.97 (7H, m), 3.55-3.65 (4H, m), 4.02 (1H, d), 4.14-4.23 (3H, m), 4.43 (1H, t), 5.05-5.15 (1H, m), 7.10 (2H, d), 7.72 (2H, d), 7.94 (1H, dd), 8.12 (1H, d), 8.42 (1H, s), 8.77 (1H, s). Mass Spectrum: m/z (ES+), [M+H]+=487.

### Example 17

(Free base) NMR Spectrum: ¹H NMR (500 MHz, CDCl₃) δ 1.46 (2H, s), 1.62 (4H, d), 1.97-2.09 (2H, m), 2.43 (5H, dtt), 2.49-2.56 (2H, m), 2.63-2.75 (1H, m), 3.62 (3H, s), 3.91-4.04 (1H, m), 4.09 (2H, t), 4.24-4.35 (2H, m), 4.43 (1H, td), 5.76-5.95 (1H, m), 7.01-7.08 (2H, m), 7.7-7.77 (2H, m), 7.89 (1H, dd), 8.19 (1H, d), 8.53 (1H, d), 8.71 (1H, s). (Methanesulfonic acid salt) NMR Spectrum: ¹H NMR (500 MHz, DMSO-d6) δ 1.29-1.51 (1H, m), 1.61-1.71 (3H, m), 1.84 (2H, d), 2.11-2.21 (2H, m), 2.29 (3H, s), 2.37-2.44 (1H, m), 2.86-2.98 (1H, m), 3.23 (2H, dt), 3.50 (2H, d), 3.54 (3H, s), 3.86-3.95 (1H, m), 4.11-4.21 (4H, m), 4.27 (1H, td), 5.76-5.91 (1H, m), 7.08-7.14 (2H, m), 7.83-7.88 (2H, m), 8.02 (1H, d), 8.14 (1H, d), 8.60 (1H, s), 8.99 (2H, s). Mass Spectrum: m/z (ES+), [M+H]+=478.

### Example 18

(Free base) NMR Spectrum: ¹H NMR (500 MHz, CDCl₃) δ 1.62 (6H, s), 1.96 (2H, d), 2.01-2.11 (2H, m), 2.48 (6H, d), 2.95-3.04 (2H, m), 3.55-3.68 (5H, m), 4.10 (2H, t), 4.25 (2H, dd), 5.12 (1H, s), 7.03-7.1 (2H, m), 7.67 (2H, d), 7.87 (1H, dd), 8.20 (1H, d), 8.42 (1H, s), 8.69 (1H, s). (Methanesulfonic acid salt) NMR Spectrum: ¹H NMR (500 MHz, DMSO-d6) δ 1.62-1.71 (2H, m), 1.84 (2H, d), 1.96 (2H, d), 2.12-2.21 (2H, m), 2.30 (3H, s), 2.66-2.78 (1H, m), 2.87-2.98 (1H, m), 3.23 (2H, dt), 3.47-3.61 (7H, m), 4.05-4.12 (2H, m), 4.15 (2H, t), 5.16-5.26 (1H, m), 7.15 (2H, d), 7.82-7.87 (2H, m), 8.13 (1H, s), 8.20 (1H, d), 8.48 (1H, s), 9.00 (1H, s), 9.08 (1H, s). Mass Spectrum: m/z (ES+), [M+H]+=501.

### Example 19

(Free base) NMR Spectrum: ¹H NMR (300 MHz, MeOH-d4) δ 1.79-1.91 (4H, m), 1.91-2.01 (1H, m), 2.01-2.16 (2H, m), 2.23-2.41 (3H, m), 2.54-2.72 (6H, m), 2.72-2.83 (2H, m), 3.38 (3H, s), 3.58 (3H, s), 4.07-4.23 (3H, m), 5.57-5.75 (1H, m), 7.02-7.14 (2H, m), 7.64-7.76 (2H, m), 7.92 (1H, dd), 8.11 (1H, d), 8.39 (1H, d), 8.75 (1H, s). (Methanesulfonic acid salt) NMR Spectrum: ¹H NMR (300 MHz, MeOH-d4) δ 1.87-2.02 (1H, m), 2.06-2.18 (4H, m), 2.18-2.38 (5H, m), 2.42-2.67 (2H, m), 2.71 (3H, s), 3.37 (3H, s), 3.41-3.51 (6H, m), 3.54 (3H, s), 4.08-4.25 (3H, m), 5.49-5.66 (1H, m), 7.03-7.15 (2H, m), 7.61-7.73 (2H, m), 7.84 (1H, dd), 8.04 (1H, d), 8.29 (1H, d), 8.69 (1H, s). Mass Spectrum: m/z (ES+), [M+H]+=501.

### Example 20

(Free base) NMR Spectrum: ¹H NMR (300 MHz, MeOH-d4) δ1.79-1.91 (4H, m), 1.91-2.01 (1H, m), 2.01-2.16 (2H, m), 2.23-2.41 (3H, m), 2.54-2.72 (6H, m), 2.72-2.83 (2H, m), 3.38 (3H, s), 3.58 (3H, s), 4.07-4.23 (3H, m), 5.57-5.75 (1H, m), 7.02-7.14 (2H, m), 7.64-7.76 (2H, m), 7.92 (1H, dd), 8.11 (1H, d), 8.39 (1H, d), 8.75 (1H, s). (Methanesulfonic acid salt) NMR Spectrum: ¹H NMR (300 MHz, MeOH-d4) δ1.87-2.02 (1H, m), 2.06-2.18 (4H, m), 2.18-2.38 (5H, m), 2.42-2.67 (2H, m), 2.71 (3H, s), 3.37 (3H, s), 3.41-3.51 (6H, m), 3.54 (3H, s), 4.08-4.25 (3H, m), 5.49-5.66 (1H, m), 7.03-7.15 (2H, m), 7.61-7.73 (2H, m), 7.84 (1H, dd), 8.04 (1H, d), 8.29 (1H, d), 8.69 (1H, s). Mass Spectrum: m/z (ES+), [M+H]+=501.

### Example 21

(Free base) NMR Spectrum: ¹H NMR (500 MHz, DMSO-d6) δ 1.87 (2H, p), 2.15 (6H, s), 2.37 (2H, t), 2.51-2.61 (2H, m), 3.15-3.28 (5H, m), 3.48 (3H, s), 4.07 (2H, t), 4.21 (1H, s), 5.31-5.69 (1H, m), 7.09 (2H, d), 7.64-7.81 (2H, m), 7.88 (1H, dd), 8.06 (1H, d), 8.21 (1H, d), 8.83 (1H, s). (Methanesulfonic acid salt) NMR Spectrum: ¹H NMR (500 MHz, DMSO-d6) δ 2.07-2.22 (2H, m), 2.30 (3H, s), 2.56 (2H, ddd), 2.84 (6H, s), 3.17-3.28 (7H, m), 3.50 (3H, s), 4.14 (2H, t), 4.22 (1H, tt), 5.54 (1H, ddd), 7.04-7.27 (2H, m), 7.73-7.84 (2H, m), 7.90 (1H, dd), 8.09 (1H, d), 8.24 (1H, d), 8.86 (1H, s), 9.34 (1H, s). Mass Spectrum: m/z (ES+), [M+H]+=461.

### Example 22

(Free base) NMR Spectrum: ¹H NMR (300 MHz, MeOH-d4) δ 1.41-1.52 (2H, m), 1.87-1.99 (4H, m), 2.05-2.20 (4H, m), 2.35 (2H, d), 2.59-2.77 (2H, m), 2.79-2.89 (4H, m), 2.89-2.98 (2H, m), 3.35-3.47 (4H, m), 3.58 (3H, s), 4.15 (2H, t), 4.95 (1H, s), 7.12 (2H, d), 7.72 (2H, d), 7.94 (1H, dd), 8.13 (1H, d), 8.37 (1H, s), 8.77 (1H, s). (Methanesulfonic acid salt) NMR Spectrum: ¹H NMR (300 MHz, MeOH-d4) δ 1.39-1.53 (4H, m), 2.12-2.20 (6H, m), 2.24-2.40 (4H, m), 2.62-2.77 (5H, m), 3.17-3.22 (1H, m), 3.38-3.53 (6H, m), 3.60 (3H, s), 3.73-3.78 (1H, m), 4.23 (2H, t), 4.94-5.03 (1H, m), 7.17 (2H, d), 7.76 (2H, d), 7.97 (1H, dd), 8.15 (1H, d), 8.39 (1H, s), 8.81 (1H, s). Mass Spectrum: m/z (ES+), [M+H]+=515.

### Example 23

(Free base) NMR Spectrum: ¹H NMR (300 MHz, MeOH-d4) δ 1.79-1.99 (3H, m), 2.06-2.18 (2H, m), 2.18-2.35 (3H, m), 2.38-2.64 (2H, m), 2.64-2.74 (2H, m), 3.29 (1H, m), 3.30-3.33 (6H, m), 3.54 (3H, s), 4.02-4.18 (3H, m), 5.42-5.60 (1H, m), 6.99-7.11 (2H, m), 7.49-7.61 (2H, m), 7.70 (1H, d), 8.20 (1H, d), 8.72 (1H, s). (Methanesulfonic acid salt) NMR Spectrum: ¹H NMR (300 MHz, MeOH-d4) δ 1.87-1.99 (1H, m), 2.04-2.38 (5H, m), 2.41-2.68 (4H, m), 2.74 (3H, s), 3.34 (3H, s), 3.47 (2H, t), 3.58 (3H, s), 4.09-4.29 (7H, m), 5.52-5.64 (1H, m), 7.11 (2H, d), 7.64 (2H, dd), 7.78 (1H, d), 8.29 (1H, d), 8.79 (1H, s). Mass Spectrum: m/z (ES+), [M+H]+=505.

### Example 24

(Free base) NMR Spectrum: ¹H NMR (300 MHz, MeOH-d4) δ 1.79-1.99 (3H, m), 2.06-2.18 (2H, m), 2.18-2.35 (3H, m), 2.38-2.64 (2H, m), 2.64-2.74 (2H, m), 3.29 (1H, s), 3.30-3.33 (6H, m), 3.54 (3H, s), 4.02-4.18 (3H, m), 5.42-5.60 (1H, m), 6.99-7.11 (2H, m), 7.49-7.61 (2H, m), 7.70 (1H, d), 8.20 (1H, d), 8.72 (1H, s). (Methanesulfonic acid salt) NMR Spectrum: ¹H NMR (300 MHz, MeOH-d4) δ 1.87-1.99 (1H, m), 2.04-2.38 (5H, m), 2.41-2.68 (4H, m), 2.74 (3H, s), 3.34 (3H, s), 3.47 (2H, t), 3.58 (3H, s), 4.09-4.29 (7H, m), 5.57 (1H, m), 7.08-7.20 (2H, d), 7.58-7.70 (2H, dd), 7.78 (1H, d), 8.29 (1H, d), 8.79 (1H, s). Mass Spectrum: m/z (ES+), [M+H]+=505.

### Example 25

(Free base) NMR Spectrum: ¹H NMR (300 MHz, CDCl₃) δ 1.54-1.75 (2H, m), 1.73-1.97 (2H, m), 2.00-2.11 (2H, m), 2.17-2.27 (2H, m), 2.33 (6H, s), 2.56 (2H, t), 2.68-3.02 (2H, m), 3.08-3.23 (3H, m), 3.57 (1H, s), 3.63 (3H, s), 4.13 (2H, t), 4.94-5.01 (1H, m), 7.08 (2H, d), 7.66-7.74 (2H, m), 7.84 (1H, dd), 8.20 (1H, d), 8.42 (1H, br), 8.71 (1H, s). (Methanesulfonic acid salt) NMR Spectrum: ¹H NMR (300 MHz, MeOH-d4) δ 1.67 (2H, t), 1.72-1.94 (2H, m), 2.18-2.37 (4H, m), 2.71 (3H, s), 2.78-2.98 (2H, m), 2.99 (6H, s), 3.21-3.31 (3H, m), 3.42 (2H, d), 3.58 (1H, s), 3.62 (3H, s), 4.22 (2H, t), 4.95-5.01 (1H, m), 7.15 (2H, d), 7.76 (2H, d), 7.95 (1H, d), 8.14 (1H, d), 8.53-8.62 (1H, br), 8.84 (1H, s). Exchangeable missing. Mass Spectrum: m/z (ES+), [M+H]+=489.

### Example 26

(Free base) NMR Spectrum: ¹H NMR (300 MHz, CDCl₃) δ 1.37-1.57 (2H, m), 1.95-2.09 (2H, m), 2.14 (2H, d), 2.25-2.40 (8H, m), 2.53-2.78 (4H, m), 3.37-3.47 (4H, m), 3.58 (3H, s), 4.12 (2H, t), 4.90-5.02 (1H, m), 7.11 (2H, d), 7.71 (2H, d), 7.94 (1H, dd), 8.13 (1H, d), 8.37 (1H, s), 8.76 (1H, s). (Methanesulfonic acid salt) NMR Spectrum: ¹H NMR (300 MHz, MeOH-d4) δ 1.36-1.53 (2H, m), 2.16 (2H, d), 2.22-2.41 (4H, m), 2.60-2.76 (5H, m), 2.99 (6H, s), 3.34-3.48 (6H, m), 3.59 (3H, s), 4.22 (2H, t), 4.90-5.05 (1H, m), 7.17 (2H, d), 7.76 (2H, d), 8.01 (1H, dd), 8.16 (1H, d), 8.41 (1H, s), 8.84 (1H, s). Exchangeable missing. Mass Spectrum: m/z (ES+), [M+H]+=489.

### Example 27

(Free base) NMR Spectrum: ¹H NMR (300 MHz, CDCl₃) δ 1.59-1.66 (2H, m), 1.69-1.89 (2H, m), 1.96-2.12 (4H, m), 2.16-2.36 (4H, m), 2.82 (3H, br), 3.07-3.19 (7H, m), 3.51 (1H, s), 3.59 (3H, s), 4.13 (2H, t), 4.92 (1H, br), 5.64 (1H, br), 6.97 (2H, d), 7.68 (2H, d), 7.81 (1H, dd), 8.18 (1H, d), 8.58 (1H, s), 8.70 (1H, s). Mass Spectrum: m/z (ES+)[M+H]+=515. (Methanesulfonic acid salt) NMR Spectrum: ¹H NMR (300 MHz, MeOH-d4) δ 1.58-1.80 (4H, m), 2.11-2.18 (4H, m), 2.20-2.26 (2H, m), 2.26-2.37 (2H, m), 2.71 (3H, s), 2.72-2.93 (3H, m), 2.93-3.26 (3H, m), 3.39-3.54 (5H, m), 3.54-3.62 (4H, m), 4.21 (2H, t), 4.81-4.96 (1H, m), 7.11 (2H, d), 7.70 (2H, d), 7.87 (1H, d), 8.08 (1H, d), 8.44-8.51 (1H, m), 8.77 (1H, s). Mass Spectrum: m/z (ES+)[M+H]+=515.

### Example 28

(Free base) NMR Spectrum: ¹H NMR (400 MHz, MeOH-d4) δ 1.47-1.55 (1H, m), 1.77-1.89 (2H, m), 1.98 (1H, d), 2.04-2.18 (3H, m), 2.34 (1H, d), 2.44 (6H, s), 2.52-2.64 (1H, m), 2.69-2.77 (2H, m), 2.77-2.85 (1H, m), 3.39 (3H, s), 3.58 (3H, s), 3.83 (1H, s), 4.14 (2H, t), 5.32-5.43 (1H, m), 7.11 (2H, d), 7.78 (2H, d), 7.95 (1H, dd), 8.12 (1H, d), 8.60 (1H, d), 8.75 (1H, s). Mass Spectrum: m/z (ES+)[M+H]+=489. (Methanesulfonic acid salt) NMR Spectrum: ¹H NMR (300 MHz, MeOD) δ 1.44-1.57 (1H, m), 1.76-1.92 (2H, m), 1.98 (1H, d), 2.12 (1H, d), 2.21-2.39 (3H, m), 2.50-2.67 (1H, m), 2.71 (3H, s), 2.73-2.83 (1H, m), 2.99 (6H, s), 3.34-3.48 (5H, m), 3.58 (3H, s), 3.83 (1H, s), 4.22 (2H, t), 5.28-5.45 (1H, m), 7.14 (2H, d), 7.79 (2H, d), 7.97 (1H, dd), 8.13 (1H, d), 8.61 (1H, d), 8.78 (1H, s). Mass Spectrum: m/z (ES+)[M+H]+=489.

### Example 29

(Free base) NMR Spectrum: ¹H NMR (400 MHz, MeOH-d4) δ 1.49 (1H, t), 1.76-1.90 (2H, m), 1.97 (1H, d), 2.00-2.10 (2H, m), 2.14 (1H, d), 2.28-2.36 (1H, m), 2.37 (6H, s), 2.51-2.61 (1H, m), 2.61-2.69 (2H, m), 2.72-2.86 (1H, m), 3.38 (3H, s), 3.57 (3H, s), 3.83 (1H, s), 4.13 (2H, t), 5.30-5.41 (1H, m), 7.10 (2H, d), 7.76 (2H, d), 7.94 (1H, dd), 8.10 (1H, d), 8.57 (1H, s), 8.74 (1H, s). Mass Spectrum: m/z (ES+)[M+H]+=489. (Methanesulfonic acid salt) NMR Spectrum: ¹H NMR (300 MHz, MeOD) δ 1.50 (1H, t), 1.76-1.92 (2H, m), 1.98 (1H, d), 2.12 (1H, d), 2.21-2.39 (3H, m), 2.51-2.67 (1H, m), 2.71 (3H, s), 2.74-2.84 (1H, m), 2.98 (6H, s), 3.37 (3H, s), 3.39-3.48 (2H, m), 3.58 (3H, s), 3.83 (1H, s), 4.22 (2H, t), 5.30-5.45 (1H, m), 7.14 (2H, d), 7.80 (2H, d), 7.97 (1H, dd), 8.13 (1H, d), 8.61 (1H, d), 8.78 (1H, s). Mass Spectrum: m/z (ES+)[M+H]+=489.

### Example 30

(Free base) NMR Spectrum: ¹H NMR (300 MHz, MeOH-d4) δ 1.50 (1H, t), 1.75-1.82 (2H, m), 1.84-2.02 (5H, m), 2.02-2.18 (3H, m), 2.31 (1H, d), 2.56 (1H, t), 2.68-2.76 (4H, m), 2.76-2.88 (3H, m), 3.37 (3H, s), 3.57 (3H, s), 3.82 (1H, s), 4.13 (2H, t), 5.28-5.39 (1H, m), 7.09 (2H, d), 7.76 (2H, d), 7.93 (1H, dd), 8.10 (1H, d), 8.57 (1H, s), 8.73 (1H, s). Mass Spectrum: m/z (ES+)[M+H]+=515. (Methanesulfonic acid salt) NMR Spectrum: ¹H NMR (300 MHz, MeOH-d4) δ 1.49 (1H, t), 1.71-1.91 (2H, m), 1.97 (1H, d), 2.11-2.17 (5H, m), 2.21-2.37 (3H, m), 2.50-2.66 (1H, m), 2.71 (3H, s), 2.73-2.83 (1H, m), 2.99-3.30 (2H, m), 3.37 (3H, s), 3.42-3.54 (2H, m), 3.57 (3H, s), 3.57-3.79 (2H, m), 3.83 (1H, s), 4.22 (2H, t), 5.28-5.43 (1H, m), 7.13 (2H, d), 7.79 (2H, d), 7.95 (1H, dd), 8.12 (1H, d), 8.59 (1H, d), 8.77 (1H, s). Mass Spectrum: m/z (ES+)[M+H]+=515.

### Example 31

(Free base) NMR Spectrum: ¹H NMR (300 MHz, MeOH-d4) δ 1.40-1.56 (1H, m), 1.70-1.82 (1H, m), 1.82-1.91 (5H, m), 1.97 (1H, d), 2.02-2.18 (3H, m), 2.32 (1H, d), 2.49-2.64 (1H, m), 2.64-2.71 (4H, m), 2.71-2.83 (3H, m), 3.38 (3H, s), 3.57 (3H, s), 3.82 (1H, s), 4.13 (2H, t), 5.29-5.39 (1H, m), 7.09 (2H, d), 7.76 (2H, d), 7.94 (1H, dd), 8.11 (1H, d), 8.58 (1H, d), 8.74 (1H, s). Mass Spectrum: m/z (ES+)[M+H]+=515. (Methanesulfonic acid salt) NMR Spectrum: ¹H NMR (300 MHz, MeOH-d4) δ 1.42-1.58 (1H, m), 1.76-1.92 (2H, m), 1.98 (1H, d), 2.06-2.18 (5H, m), 2.21-2.39 (3H, m), 2.50-2.66 (1H, m), 2.71 (3H, s), 2.72-2.84 (1H, m), 3.02-3.28 (2H, m), 3.37 (3H, s), 3.40-3.54 (2H, m), 3.58 (3H, s), 3.57-3.80 (2H, m), 3.83 (1H, s), 4.22 (2H, t), 5.30-5.44 (1H, m), 7.14 (2H, d), 7.79 (2H, d), 7.97 (1H, dd), 8.13 (1H, d), 8.61 (1H, d), 8.78 (1H, s). Mass Spectrum: m/z (ES+)[M+H]+=515.

### Example 32

(Free base) NMR Spectrum: ¹H NMR (300 MHz, MeOH-d4) δ 1.25-1.36 (1H, m), 1.44-1.58 (1H, m), 1.95-2.11 (4H, m), 2.16-2.27 (1H, m), 2.35 (6H, s), 2.38-2.51 (3H, m), 2.51-2.67 (2H, m), 3.39 (3H, s), 3.44-3.51 (1H, m), 3.57 (3H, s), 4.11 (2H, t), 4.91-4.98 (1H, m), 7.09 (2H, dd), 7.68 (2H, dd), 7.90 (1H, dd), 8.10 (1H, d), 8.29 (1H, s), 8.74 (1H, s). Mass Spectrum: m/z (ES+)[M+H]+=489. (Methanesulfonic acid salt) NMR Spectrum: ¹H NMR (300 MHz, MeOH-d4) δ 1.26-1.37 (1H, m), 1.50-1.61 (1H, m), 2.00-2.12 (2H, m), 2.17-2.38 (3H, m), 2.37-2.57 (3H, m), 2.71 (3H, s), 2.99 (6H, s), 3.40 (3H, s), 3.40-3.48 (3H, m), 3.61 (3H, s), 4.23 (2H, t), 4.86-4.93 (1H, m), 7.18 (2H, dd), 7.76 (2H, dd), 8.13 (1H, dd), 8.20 (1H, d), 8.44 (1H, br), 8.96 (1H, s). Mass Spectrum: m/z (ES+)[M+H]+=489.

### Example 33

(Free base) NMR Spectrum: ¹H NMR (300 MHz, MeOH-d4) δ 1.32 (1H, m), 1.54 (1H, m), 1.96-2.12 (4H, m), 2.22 (1H, d), 2.35 (6H, s), 2.37-2.53 (3H, m), 2.57-2.68 (2H, m), 3.35-3.48 (4H, m), 3.59 (3H, s), 4.12 (2H, t), 4.63 (1H, s), 7.05-7.16 (2H, d), 7.63-7.76 (2H, d), 7.93 (1H, dd), 8.13 (1H, d), 8.35 (1H, s), 8.77 (1H, s). Mass Spectrum: m/z (ES+)[M+H]+=489. (Methanesulfonic acid salt) NMR Spectrum: ¹H NMR (300 MHz, MeOH-d4) δ 1.27-1.42 (1H, m), 1.44-1.59 (1H, m), 1.98-2.11 (2H, m), 2.16-2.35 (3H, m), 2.36-2.52 (3H, m), 2.71 (3H, s), 2.95 (6H, s), 3.32-3.44 (6H, m), 3.59 (3H, s), 4.21 (2H, t), 4.92 (1H, s), 7.09-7.20 (2H, m), 7.67-7.79 (2H, m), 7.92 (1H, dd), 8.13 (1H, d), 8.35 (1H, s), 8.78 (1H, s). Mass Spectrum: m/z (ES+)[M+H]+=489.

### Example 34

(Free base) NMR Spectrum: ¹H NMR (300 MHz, MeOH-d4) δ 1.97-2.23 (5H, m), 2.39 (6H, s), 2.50 (2H, m), 2.58-2.73 (3H, m), 3.23 (3H, s), 3.59 (3H, s), 3.99-4.06 (1H, m), 4.11 (2H, t), 5.31-5.47 (1H, m), 7.01-7.13 (2H, m), 7.64-7.75 (2H, m), 7.88 (1H, dd), 8.09 (1H, d), 8.42 (1H, s), 8.76 (1H, s). Mass Spectrum: m/z (ES+)[M+H]+=475. (Methanesulfonic acid salt) NMR Spectrum: ¹H NMR (300 MHz, MeOH-d4) δ 2.02-2.22 (3H, m), 2.21-2.37 (2H, m), 2.43-2.56 (2H, m), 2.57-2.74 (4H, m), 2.99 (6H, s), 3.22 (3H, s), 3.36-3.48 (2H, m), 3.59 (3H, s), 3.96-4.10 (1H, m), 4.21 (2H, t), 5.31-5.46 (1H, m), 7.05-7.16 (2H, m), 7.65-7.77 (2H, m), 7.87 (1H, dd), 8.08 (1H, d), 8.42 (1H, d), 8.77 (1H, s). Mass Spectrum: m/z (ES+)[M+H]+=475.

### Example 35

(Free base) NMR Spectrum: ¹H NMR (300 MHz, MeOH-d4) δ 1.97-2.23 (5H, m), 2.39 (6H, s), 2.42-2.59 (2H, m), 2.58-2.73 (3H, m), 3.23 (3H, s), 3.59 (3H, s), 3.99-4.06, 4.11 (2H, t), (1H, m), 5.31-5.47 (1H, m), 7.01-7.13 (2H, m), 7.64-7.75 (2H, m), 7.88 (1H, dd), 8.09 (1H, d), 8.42 (1H, s), 8.76 (1H, s). Mass Spectrum: m/z (ES+)[M+H]+=475. (Methanesulfonic acid salt) NMR Spectrum: ¹H NMR (300 MHz, MeOH-d4) δ 2.02-2.22 (3H, m), 2.21-2.37 (2H, m), 2.43-2.56 (2H, m), 2.57-2.74 (4H, m), 2.99 (6H, s), 3.22 (3H, s), 3.36-3.48 (2H, m), 3.59 (3H, s), 3.96-4.10 (1H, m), 4.21 (2H, t), 5.31-5.46 (1H, m), 7.05-7.16 (2H, m), 7.65-7.77 (2H, m), 7.87 (1H, dd), 8.08 (1H, d), 8.42 (1H, d), 8.77 (1H, s). Mass Spectrum: m/z (ES+)[M+H]+=475.

### Example 36

(Free base) NMR Spectrum: ¹H NMR (300 MHz, MeOH-d4) δ 1.94-2.21 (5H, m), 2.34 (6H, s), 2.39-2.52 (2H, m), 2.53-2.74 (3H, m), 3.12 (3H, s), 3.61 (3H, s), 3.91-4.06 (1H, m), 4.12 (2H, t), 5.29-5.48 (1H, m), 7.02-7.14 (2H, m), 7.54-7.65 (2H, m), 7.78 (1H, d), 8.43 (1H, d), 8.81 (1H, s). Mass Spectrum: m/z (ES+)[M+H]+=493. (Methanesulfonic acid salt) NMR Spectrum: ¹H NMR (300 MHz, MeOH-d4) δ 1.96-2.21 (3H, m), 2.21-2.37 (2H, m), 2.36-2.54 (2H, m), 2.54-2.74 (4H, m), 2.99 (6H, s), 3.10 (3H, s), 3.36-3.48 (2H, m), 3.61 (3H, s), 3.91-4.05 (1H, m), 4.22 (2H, t), 5.29-5.48 (1H, m), 7.07-7.18 (2H, m), 7.57-7.67 (2H, m), 7.79 (1H, d), 8.45 (1H, d), 8.84 (1H, s). Mass Spectrum: m/z (ES+)[M+H]+=493.

### Example 37

(Free base) NMR Spectrum: ¹H NMR (300 MHz, MeOH-d4) δ 1.94-2.21 (5H, m), 2.34 (6H, s), 2.39-2.52 (2H, m), 2.53-2.74 (3H, m), 3.12 (3H, s), 3.61 (3H, s), 3.91-4.06 (1H, m), 4.12 (2H, t), 5.29-5.48 (1H, m), 7.02-7.14 (2H, m), 7.54-7.65 (2H, m), 7.78 (1H, d), 8.43 (1H, d), 8.81 (1H, s). Mass Spectrum: m/z (ES+)[M+H]+=493. (Methanesulfonic acid salt) NMR Spectrum: ¹H NMR (300 MHz, MeOH-d4) δ 1.92-2.21 (3H, m), 2.19-2.37 (2H, m), 2.36-2.54 (2H, m), 2.54-2.74 (4H, m), 2.99 (6H, s), 3.10 (3H, s), 3.36-3.48 (2H, m), 3.61 (3H, s), 3.91-4.05 (1H, m), 4.22 (2H, t), 5.30-5.49 (1H, m), 7.07-7.19 (2H, m), 7.56-7.67 (2H, m), 7.80 (1H, d), 8.46 (1H, d), 8.86 (1H, s). Mass Spectrum: m/z (ES+)[M+H]+=493.

### Example 38

(Free base) NMR Spectrum: ¹H NMR (300 MHz, MeOH-d4) δ 1.19-1.38 (1H, m), 1.40-1.60 (1H, m), 1.94-2.12 (6H, m), 2.12-2.28 (3H, m), 2.28-2.53 (3H, m), 3.11-3.26 (6H, m), 3.38 (3H, s), 3.38-3.48 (1H, m), 3.53 (3H, s), 4.15 (2H, t), 4.80-4.87 (1H, m), 7.02-7.14 (2H, m), 7.58-7.70 (2H, m), 7.83 (1H, dd), 8.04 (1H, d), 8.56 (1H, s), 8.69 (1H, s). Mass Spectrum: m/z (ES+)[M+H]+=515. (Methanesulfonic acid salt) NMR Spectrum: 1H NMR (300 MHz, MeOH-d4) δ 1.27-1.38 (1H, m), 1.49-1.60 (1H, m), 1.99-2.12 (4H, m), 2.16-2.37 (5H, m), 2.37-2.54 (3H, m), 2.71 (3H, s), 3.07-3.18 (2H, m), 3.39 (3H, s), 3.40-3.54 (3H, m), 3.60 (3H, s), 3.71-3.77 (2H, m), 4.22 (2H, t), 4.90-5.00 (1H, m), 7.15 (2H, d), 7.75 (2H, d), 7.98 (1H, dd), 8.16 (1H, d), 8.39 (1H, s), 8.84 (1H, s). Mass Spectrum: m/z (ES+)[M+H]+=515.

### Example 39

(Free base) NMR Spectrum: ¹H NMR (300 MHz, MeOH-d4) δ 1.22-1.37 (1H, m), 1.44-1.64 (1H, m), 1.81-1.97 (4H, m), 1.97-2.27 (5H, m), 2.34-2.57 (3H, m), 2.66-2.86 (6H, m), 3.39 (4H, s), 3.58 (3H, s), 4.13 (2H, t), 4.90 (1H, s), 7.04-7.16 (2H, d), 7.64-7.75 (2H, d), 7.92 (1H, dd), 8.12 (1H, d), 8.33 (1H, s), 8.76 (1H, s). Mass Spectrum: m/z (ES+)[M+H]+=515. (Methanesulfonic acid salt) NMR Spectrum: ¹H NMR (400 MHz, MeOH-d4) δ 1.26-1.41 (1H, m), 1.48-1.63 (1H, m), 2.01-2.11 (3H, m), 2.17-2.36 (6H, m), 2.40-2.53 (3H, m), 2.72 (3H, s), 3.17-3.22 (2H, m), 3.33-3.53 (6H, m), 3.61 (3H, s), 3.73-3.78 (2H, m), 4.23 (2H, t), 5.04-5.22 (1H, m), 7.13-7.23 (2H, m), 7.71-7.79 (2H, m), 7.98 (1H, dd), 8.16 (1H, d), 8.37 (1H, s), 8.83 (1H, d). Mass Spectrum: m/z (ES+)[M+H]+=515.

### Example 40

(Free base) NMR Spectrum: ¹H NMR (500 MHz, CDCl₃) δ 1.46 (2H, s), 1.92 (1H, d), 2.04 (2H, s), 2.24-2.4 (4H, m), 2.4-2.62 (6H, m), 2.68-2.78 (1H, m), 3.37 (3H, s), 3.58 (3H, s), 4.09 (2H, t), 4.18 (1H, dd), 5.61 (1H, s), 7.01-7.08 (2H, m), 7.61-7.67 (2H, m), 7.84 (1H, dd), 8.18 (1H, d), 8.33 (1H, d), 8.67 (1H, s) (4 protons disguised under water peak at 1.5 ppm). Mass Spectrum: m/z (ES+)[M+H]+=515. (Methanesulfonic acid salt) NMR Spectrum: ¹H NMR (500 MHz, CDCl₃) δ 1.85-2.15 (6H, m), 2.2-2.46 (6H, m), 2.51-2.62 (1H, m), 2.68-2.78 (1H, m), 2.83 (3H, s), 2.84-3.36 (6H, m), 3.37 (3H, s), 3.58 (3H, s), 4.17 (3H, t), 5.61 (1H, p), 6.98-7.05 (2H, m), 7.61-7.68 (2H, m), 7.83 (1H, dd), 8.19 (1H, d), 8.33 (1H, d), 8.68 (1H, s). Mass Spectrum: m/z (ES+)[M+H]+=515.

### Example 41

(Free base) NMR Spectrum: ¹H NMR (300 MHz, MeOH-d4) δ 1.90-2.06 (4H, m), 2.08-2.24 (2H, m), 2.83-2.91 (2H, m), 2.95-3.12 (8H, m), 3.30 (3H, s), 3.52 (3H, s), 3.80-3.96 (1H, m), 4.14 (2H, t), 4.86-5.04 (1H, m), 7.01-7.13 (2H, m), 7.62-7.73 (2H, m), 7.82 (1H, dd), 8.02 (1H, d), 8.26 (1H, s), 8.67 (1H, s). Mass Spectrum: m/z (ES+)[M+H]+=487. (Methanesulfonic acid salt) NMR Spectrum: ¹H NMR (300 MHz, MeOH-d4) δ 2.15 (4H, s), 2.22-2.37 (2H, m), 2.71 (3H, s), 2.87-3.17 (5H, m), 3.32 (3H, s), 3.41-3.52 (3H, m), 3.52-3.82 (5H, m), 3.83-3.99 (1H, m), 4.22 (2H, t), 4.96-5.14 (1H, m), 7.07-7.19 (2H, m), 7.69-7.81 (2H, m), 7.91 (1H, dd), 8.09 (1H, d), 8.38 (1H, d), 8.77 (1H, s). Mass Spectrum: m/z (ES+)[M+H]+=487.

### Example 42

(Free base) NMR Spectrum: ¹H NMR (500 MHz, DMSO-d6) δ 1.59-1.77 (4H, m), 1.91 (2H, p), 2.39-2.46 (4H, m), 2.52-2.61 (4H, m), 3.15-3.27 (5H, m), 3.49 (3H, s), 4.08 (2H, t), 4.21 (1H, dt), 5.42-5.64 (1H, m), 6.99-7.22 (2H, m), 7.67-7.82 (2H, m), 7.88 (1H, dd), 8.07 (1H, d), 8.22 (1H, d), 8.83 (1H, s). Mass Spectrum: m/z (ES+)[M+H]+=487. (Methanesulfonic acid salt) NMR Spectrum: ¹H NMR (500 MHz, DMSO-d6) δ 1.78-1.95 (2H, m), 1.96-2.1 (2H, m), 2.08-2.23 (2H, m), 2.30 (3H, s), 2.56 (2H, ddd), 3.07 (2H, d), 3.15-3.28 (5H, m), 3.32-3.38 (2H, m), 3.50 (3H, s), 3.60 (2H, s), 4.15 (2H, t), 4.22 (1H, tt), 5.34-5.8 (1H, m), 7.03-7.29 (2H, m), 7.72-7.86 (2H, m), 7.92 (1H, dd), 8.10 (1H, d), 8.25 (1H, d), 8.88 (1H, s), 9.46 (1H, s). Mass Spectrum: m/z (ES+)[M+H]+=487.

### Example 43

(Free base) NMR Spectrum: ¹H NMR (500 MHz, CDCl₃) δ 1.46 (2H, s), 1.95-2.13 (2H, m), 2.37-2.65 (6H, m), 2.92-3.01 (2H, m), 3.14-3.24 (2H, m), 3.31 (3H, s), 3.58 (3H, s), 3.83-3.93 (1H, m), 4.10 (2H, t), 4.87-4.98 (1H, m), 7.02-7.09 (2H, m), 7.61-7.68 (2H, m), 7.82 (1H, dd), 8.18 (1H, d), 8.30 (1H, d), 8.68 (1H, s). (4 protons disguised by water at 1.5 ppm). Mass Spectrum: m/z (ES+)[M+H]+=501. (Methanesulfonic acid salt) NMR Spectrum: ¹H NMR (500 MHz, DMSO-d6) δ 1.39 (1H, d), 1.58-1.77 (3H, m), 1.84 (2H, d), 2.13-2.2 (2H, m), 2.29 (3H, s), 2.82 (2H, d), 2.87-3.05 (4H, m), 3.19 (3H, s), 3.21-3.26 (2H, m), 3.50 (5H, s), 3.76-3.91 (1H, m), 4.14 (2H, t), 5.01-5.18 (1H, m), 7.12 (2H, d), 7.83 (2H, d), 7.93 (1H, d), 8.11 (1H, d), 8.39 (1H, s), 8.90 (1H, s), 8.97 (1H, s). Mass Spectrum: m/z (ES+)[M+H]+=501.

### Example 44

(Free base) NMR Spectrum: ¹H NMR (500 MHz, DMSO-d6) δ 1.79-1.98 (4H, m), 2.15 (6H, s), 2.37 (2H, t), 2.71 (2H, qd), 3.50 (3H, s), 3.51-3.64 (2H, m), 4.07 (4H, t), 5.11 (1H, t), 7.03-7.18 (2H, m), 7.71-7.85 (2H, m), 7.92 (1H, dd), 8.09 (1H, d), 8.39 (1H, s), 8.85 (1H, s). Mass Spectrum: m/z (ES+)[M+H]+=461. (Methanesulfonic acid salt) NMR Spectrum: ¹H NMR (500 MHz, DMSO-d6) 1.92 (2H, d), 2.07-2.21 (2H, m), 2.30 (3H, s), 2.72 (2H, qd), 2.84 (6H, s), 3.2-3.27 (2H, m), 3.51 (3H, s), 3.56 (2H, t), 4.08 (2H, dd), 4.14 (2H, t), 5.03-5.27 (1H, m), 7.03-7.23 (2H, m), 7.76-7.89 (2H, m), 7.97 (1H, d), 8.12 (1H, d), 8.42 (1H, s), 8.90 (1H, s), 9.33 (1H, s). Mass Spectrum: m/z (ES+)[M+H]+=461.

The preparation of the appropriate bromo intermediates required for Examples 7-44 are described below.

### Intermediate D1: 8-Bromo-3-methyl-1-(oxan-4-yl)imidazo[5,4-c]quinolin-2-one

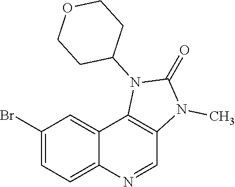 [SMILES: Cn1c(=O)n(C2CCOCC2)c2c3cc(Br)ccc3ncc21]

A solution of sodium hydroxide (10.34 g, 258.48 mmol) in water (900 mL) was added to a stirred mixture of 8-bromo-1-(oxan-4-yl)-3H-imidazo[4,5-c]quinolin-2-one (60.0 g, 172.32 mmol), iodomethane (48.9 g, 344.63 mmol) and tetrabutylammonium bromide (5.55 g, 17.23 mmol) in DCM (1500 mL) at ambient temperature under air. The resulting mixture was stirred for 16 h then the DCM removed under reduced pressure. The precipitate was collected by filtration, washed with water (200 mL) and dried under vacuum to afford the desired material (58.0 g, 93%) as a brown solid, which was used without further purification. NMR Spectrum: ¹H NMR (400 MHz, CDCl₃) δ 1.81-1.98 (2H, m), 2.82-3.00 (2H, m), 3.60 (3H, s), 3.63 (2H, td), 4.05-4.35 (2H, m), 4.93 (1H, t), 7.69 (1H, dd), 8.03 (1H, d), 8.36 (1H, s), 8.71 (1H, s). Mass Spectrum: m/z (ES+)[M+H]+=364.

On a larger scale, 8-bromo-1-(oxan-4-yl)-3H-imidazo[4,5-c]quinolin-2-one (1300 g, 3.73 mol) was charged to the vessel along with tetrabutylammonium bromide (130 g, 0.40 mol) and 2-MeTHF (20.8 L). A solution of NaOH (240 g, 6.00 mol) in water (20.8 L) was then added over 5 minutes with an observed exotherm from 18-24° C. The biphasic mixture was heated to 42-48° C. before the addition of methyl iodide (465 mL, 7.47 mol) as a solution in 2-MeTHF (930 mL). The reaction was stirred at 45° C. for 17 h at which point HPLC analysis showed 2.9% starting material and 97.1% product. The reaction mixture was combined with that of the other large scale batches for concentration in vacuo. The resulting aqueous suspension was then returned to the vessel and slurried for 1 h with the product material obtained from the development batches combined at this point. The product was then isolated by filtration, washing with water (2×12 L) before oven drying under vacuum at 40° C. In total 3479 g of 8-bromo-3-methyl-1-(oxan-4-yl)imidazo[5,4-c]quinolin-2-one was isolated. Analytical data was consistent with that obtained from previous batches.

The following intermediates were prepared in an analogous fashion from the appropriate 3H-imidazo[4,5-c]quinolin-2-one intermediate:

| Inter- | | |
|---|---|---|
| medi- | | |
| ate | Structure | Name |
| Inter- medi- ate E1 | 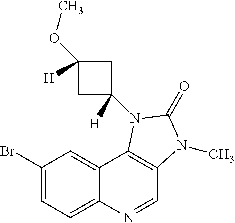 [SMILES: CO[C@H]1C[C@@H](n2c(=O)n(C)c3cnc4ccc(Br)cc4c32)C1] | 8-Bromo-1-(cis-3- methoxycyclobutyl)- 3- methylimidazo[4,5- c]quinolin-2-one |
| Inter- medi- ate F1 | 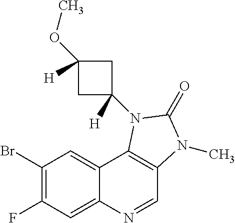 [SMILES: CO[C@H]1C[C@@H](n2c(=O)n(C)c3cnc4cc(F)c(Br)cc4c32)C1] | 8-Bromo-7-fluoro-1- (cis-3- methoxycyclobutyl)- 3- methylimidazo[4,5- c]quinolin-2-one |
| Inter- medi- ate G1 | 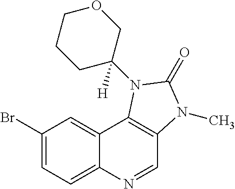 [SMILES: Cn1c(=O)n([C@H]2CCCOC2)c2c3cc(Br)ccc3ncc21] | 8-bromo-3-methyl- 1-[(3S)-oxan-3- yl]imidazo[5,4- c]quinolin-2-one |
| Inter- medi- ate H1* | 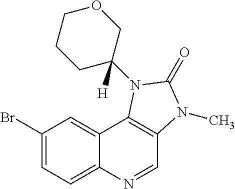 [SMILES: Cn1c(=O)n([C@@H]2CCCOC2)c2c3cc(Br)ccc3ncc21] | 8-bromo-3-methyl- 1-[(3R)-oxan-3- yl]imidazo[5,4- c]quinolin-2-one |
| Inter- medi- ate I1 | 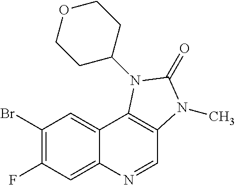 [SMILES: Cn1c(=O)n(C2CCOCC2)c2c3cc(Br)c(F)cc3ncc21] | 8-bromo-7-fluoro-3- methyl-1-(oxan-4- yl)imidazo[5,4- c]quinolin-2-one |
| Inter- medi- ate J1** | 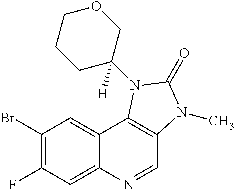 [SMILES: Cn1c(=O)n([C@H]2CCCOC2)c2c3cc(Br)c(F)cc3ncc21] | 8-bromo-7-fluoro-3- methyl-1-[(3S)- oxan-3- yl]imidazo[5,4- c]quinolin-2-one |
| Inter- medi- ate K1 | 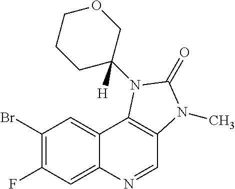 [SMILES: Cn1c(=O)n([C@@H]2CCCOC2)c2c3cc(Br)c(F)cc3ncc21] | 8-bromo-7-fluoro-3- methyl-1-[(3R)- oxan-3- yl]imidazo[5,4- c]quinolin-2-one |
| Inter- medi- ate L1 | 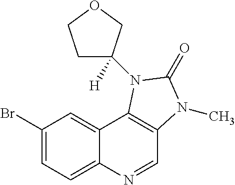 [SMILES: Cn1c(=O)n([C@H]2CCOC2)c2c3cc(Br)ccc3ncc21] | 8-bromo-3-methyl- 1-[(3S)- tetrahydrofuran-3- yl]imidazo[4,5- c]quinolin-2-one |
| Inter- medi- ate M1 | 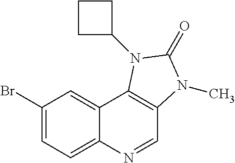 [SMILES: Cn1c(=O)n(C2CCC2)c2c3cc(Br)ccc3ncc21] | 8-bromo-1- cyclobutyl-3- methyl-imidazo[4,5- c]quinolin-2-one |

*The reaction had not proceeded to completion so additional methyl iodide, sodium hydroxide and tetrabutylammonium bromide were added and the reaction stirred a further 16-18 h.
**The reaction was stirred for 72 h at ambient temperature.

Intermediate E1:

NMR Spectrum: ¹H NMR (400 MHz, DMSO-d6) δ 2.72-2.86 (2H, m), 2.9-3.08 (2H, m), 3.22 (3H, s), 3.49 (3H, s), 3.85-3.89 (1H, m), 4.88-5.06 (1H, m), 7.74 (1H, dd), 7.98 (1H, d), 8.50 (1H, d), 8.92 (1H, s). Mass Spectrum: m/z (ES+)[M+H]+=362, 364.

Intermediate F1:

NMR Spectrum: ¹H NMR (300 MHz, DMSO-d6) δ 2.70-2.85 (2H, m), 2.93-3.07 (2H, m), 3.22 (3H, s), 3.48 (3H, s), 3.73-4.00 (1H, m), 4.86-5.15 (1H, m), 7.75-8.07 (1H, d), 8.52-8.73 (1H, d), 8.93 (1H, s). Mass Spectrum: m/z (ES+)[M+H]+=380.

Intermediate G1:

NMR Spectrum: ¹H NMR (300 MHz, DMSO-d6) δ 1.82-1.88 (2H, m), 2.09-2.15 (1H, m), 2.55-2.78 (1H, m), 3.30-3.47 (1H, m) 3.48 (3H, s), 3.92 (1H, d), 4.02-4.22 (2H, m), 4.68-4.88 (1H, m), 7.75 (1H, d), 7.99 (1H, d), 8.35 (1H, s), 8.92 (1H, s). Mass Spectrum: m/z (ES+)[M+H]+=362.2.

Intermediate H1:

NMR Spectrum: ¹H NMR (300 MHz, DMSO-d6) δ 1.80-1.86 (2H, m), 2.07-2.12 (1H, m), 2.61-2.75 (1H, m), 3.32-3.46 (1H, m), 3.47 (3H, s), 3.92-3.98 (1H, m), 4.01-4.20 (2H, m), 4.72-4.83 (1H, m), 7.76 (1H, dd), 8.00 (1H, d), 8.34 (1H, d), 8.92 (1H, s). Mass Spectrum: m/z (ES+)[M+H]+=362, 364.

Intermediate I1:

NMR Spectrum: ¹H NMR (400 MHz, DMSO-d6, 100° C.) δ 1.88 (2H, d), 2.59-2.84 (2H, m), 3.50 (3H, s), 3.60 (2H, t), 4.06 (2H, d), 4.95 (1H, s), 7.90 (1H, d), 8.56 (1H, d), 8.89 (1H, s). Mass Spectrum: m/z (ES+)[M+H]+=381.96.

Intermediate J1:

NMR Spectrum: ¹H NMR (400 MHz, DMSO-d6) δ 1.88-190 (2H, m), 2.09 (1H, d), 2.70 (1H, ddd), 3.36-3.44 (1H, m), 3.47 (3H, s), 3.94 (1H, d), 4.07 (1H, dd), 4.15 (1H, t), 4.79 (1H, ddd), 7.97 (1H, d), 8.48 (1H, d), 8.93 (1H, s). Mass Spectrum: m/z (ES+)[M+H]+=380, 382.

Intermediate K1:

NMR Spectrum: ¹H NMR (400 MHz, DMSO-d6) δ 1.86 (2H, dd), 2.11 (1H, d), 2.69 (1H, ddd), 3.37-3.45 (1H, m), 3.48 (3H, s), 3.95 (1H, d), 4.08 (1H, dd), 4.18 (1H, t), 4.80 (1H, ddd), 7.98 (1H, d), 8.50 (1H, d), 8.94 (1H, s). Mass Spectrum: m/z (ES+)[M+H]+=380, 382.

Intermediate L1:

NMR Spectrum: ¹H NMR (400 MHz, DMSO-d6) δ 2.40-2.48 (1H, m), 2.58-2.67 (1H, m), 3.63 (3H, s), 3.98-4.05 (1H, m), 4.19-4.28 (2H, m), 4.46-4.51 (1H, td), 5.68-5.76 (1H, m), 7.72 (1H, d), 8.07 (1H, d), 8.67 (1H, d), 8.76 (1H, s). Mass Spectrum: m/z (ES+)[M+H]+=348.

Intermediate M1:

NMR Spectrum: ¹H NMR (400 MHz, CDCl₃) δ 1.95-2.12 (2H, m), 2.52-2.59 (2H, m), 3.17-3.28 (2H, m), 3.59 (3H, s), 5.18-5.27 (1H, m), 7.8 (1H, d), 8.02 (1H, d), 8.37 (1H, d), 8.70 (1H, s). Mass Spectrum: m/z (ES+)[M+H]+=332.

### Intermediate N1: 8-Bromo-1-(trans-3-methoxycyclobutyl)-3-methyl-imidazo[4,5-c]quinolin-2-one

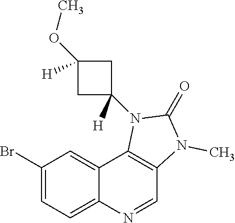 [SMILES: CO[C@H]1C[C@H](n2c(=O)n(C)c3cnc4ccc(Br)cc4c32)C1]

To a suspension of 8-bromo-1-(trans-3-hydroxycyclobutyl)-3H-imidazo[4,5-c]quinolin-2-one (1.8 g, 5.39 mmol) in DMF (20 mL) under nitrogen at RT was added NaH (60% in mineral oil) (0.75 g, 18.75 mmol) and the solution was stirred for 30 minutes. Methyl iodide (1 mL, 15.99 mmol) was added and the reaction mixture stirred at ambient temperature for one h. A second identical reaction was performed using 8-bromo-1-((trans)-3-hydroxycyclobutyl)-1H-imidazo[4,5-c]quinolin-2(3H)-one (0.5 g, 1.50 mmol), DMF (5 mL), NaH (60% in mineral oil) (0.22 g, 5.50 mmol) and methyl iodide (0.3 mL, 4.80 mmol) and the reactions combined. The combined reaction mixture was carefully quenched with water and then stirred in water for thirty minutes. The solid was filtered off, washed thoroughly with water then dried to afford the desired material as an off white solid (1.965 g, 79%). NMR Spectrum: ¹H NMR (500 MHz, DMSO-d6) δ 2.5-2.56 (2H, m), 3.11-3.21 (2H, m), 3.23 (3H, s), 3.48 (3H, s), 4.20 (1H, dt), 5.34-5.54 (1H, m), 7.72 (1H, dd), 7.95 (1H, d), 8.28 (1H, d), 8.90 (1H, s). Mass Spectrum: m/z (ES+)[M+H]+=362, 364.

The following intermediates were prepared in an analogous fashion from the appropriate 3H-imidazo[4,5-c]quinolin-2-one intermediate:

| Inter- | | |
|---|---|---|
| medi- | | |
| ate | Structure | Name |
| Inter- medi- ate O1* | 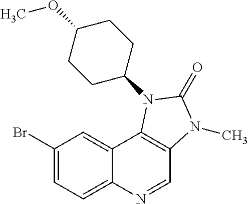 | 8-bromo-1-(trans-4- methoxycyclohexyl)- 3-methyl- imidazo[4,5- c]quinolin-2-one |
| Inter- medi- ate P1* | 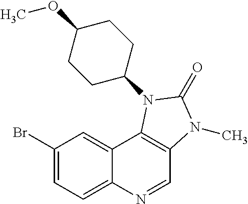 | 8-bromo-1-(cis-4- methoxycyclohexyl)- 3-methyl- imidazo[4,5- c]quinolin-2-one |
| Inter- medi- ate Q1* | 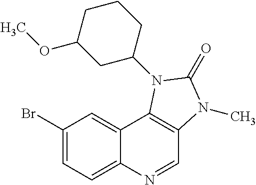 | 8-bromo-1-[(3- methoxycyclohexyl]- 3-methyl- imidazo[4,5- c]quinolin-2-one (mixture of diastereoisomers) |
| Inter- medi- ate R1** | 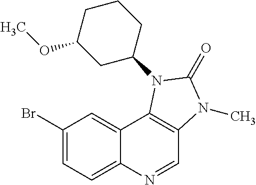 | 8-bromo-1-[(trans-3- methoxycyclohexyl]- 3-methyl- imidazo[4,5- c]quinolin-2-one (1:1 mixture of enantiomers) |
| Inter- medi- ate S1** | 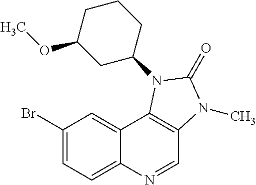 | 8-bromo-1-[(cis-3- methoxycyclohexyl]- 3-methyl- imidazo[4,5- c]quinolin-2- one—Isomer 1 |
| Inter- medi- ate T1** | 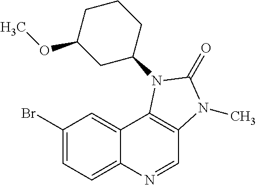 | 8-bromo-1-[(cis-3- methoxycyclohexyl]- 3-methyl- imidazo[4,5- c]quinolin-2- one—Isomer 2 |
| Inter- medi- ate U1* | 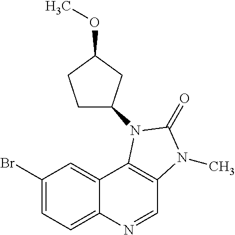 [SMILES: CO[C@@H]1CC[C@H](n2c(=O)n(C)c3cnc4ccc(Br)cc4c32)C1] | 8-bromo-1-[(cis-3- methoxycyclopentyl]- 3-methyl- imidazo[4,5- c]quinolin-2-one (1:1 mixture of isomers) |

*The reaction was stirred at 0° C. for 1 h then at ambient temperature overnight
**Intermediates R1, S1 and T1 were separated from a racemic mixture, Intermediate Q1 by Supercritical Fluid Chromatography using an SFC prep 350 machine and a CHIRALPAK AD-H SFC (5 * 25 cm, 5 μm) column (Flow rate 150 mL/min, Pressure 100 bar, Temperature 34° C., Mobile Phase A: CO₂: 50, Mobile Phase B: MeOH: 50). Intermediate R1 was eluted first followed by Intermediate S1 and finally Intermediate T1. Intermediate T1 was subsequently purified again using the SFC prep 350 machine and a CHIRALPAK AD-H SFC (5 * 25 cm, 5 μm) column (Flow rate 150 mL/min, Pressure 100 bar, Temperature 34° C., Mobile Phase A: CO₂: 60, Mobile Phase B: MeOH: 40).

Intermediate O1:

NMR Spectrum: ¹H NMR (300 MHz, CDCl₃) δ 1.40-1.60 (2H, m), 2.08 (2H, d), 2.35 (2H, d), 2.63-2.77 (2H, m), 3.33-3.44 (1H, m), 3.45 (3H, s), 3.57 (3H, s), 4.68 (1H, s), 7.70 (1H, dd), 8.05 (1H, d), 8.30 (1H, s), 8.70 (1H, s). Mass Spectrum: m/z (ES+)[M+H]+=390.

Intermediate P1:

NMR Spectrum: ¹H NMR (400 MHz, CDCl₃) δ 1.64-1.77 (4H, m), 2.21-2.32 (2H, m), 2.65 (2H, s), 3.56 (3H, s), 3.65 (4H, d), 4.98 (1H, s), 7.71 (1H, dd), 8.03 (1H, d), 8.74 (1H, s), 8.83 (1H, s). Mass Spectrum: m/z (ES+)[M+H]+=390.

Intermediate R1:

NMR Spectrum: ¹H NMR (400 MHz, CDCl₃) δ 1.40-1.63 (1H, m), 1.75-1.94 (2H, m), 2.01 (1H, d), 2.09 (1H, d), 2.32 (1H, d), 2.45-2.52 (1H, m), 2.84 (1H, d), 3.50 (3H, s), 3.57 (3H, s), 3.81-3.84 (1H, m), 5.10 (1H, t), 7.70 (1H, dd), 8.03 (1H, d), 8.66 (1H, d), 8.70 (1H, s). Mass Spectrum: m/z (ES+)[M+H]+=390.

Intermediate S1:

NMR Spectrum: ¹H NMR (400 MHz, CDCl₃) δ 1.40-1.53 (2H, m), 1.96-2.13 (2H, m), 2.22 (1H, d), 2.44-2.54 (3H, m), 3.37-3.42 (1H, m), 3.42 (3H, s), 3.60 (3H, s), 4.66 (1H, s), 7.70 (1H, dd), 8.06 (1H, d), 8.29 (1H, s), 8.73 (1H, s). Mass Spectrum: m/z (ES+)[M+H]+=390.

Intermediate T1:

NMR Spectrum: ¹H NMR (300 MHz, CDCl₃) δ 1.40-1.53 (2H, m), 1.96-2.13 (2H, m), 2.22 (1H, d), 2.44-2.54 (3H, m), 3.37-3.42 (1H, m), 3.42 (3H, s), 3.60 (3H, s), 4.66 (1H, s), 7.70 (1H, dd), 8.06 (1H, d), 8.29 (1H, s), 8.73 (1H, s). Mass Spectrum: m/z (ES+)[M+H]+=390.

Intermediate U1:

NMR Spectrum: ¹H NMR (400 MHz, CDCl₃) δ 1.89-2.04 (1H, m), 2.01-2.14 (1H, m), 2.27 (1H, t), 2.37-2.68 (3H, m), 3.47 (2H, s), 3.63 (2H, s), 4.06-4.08 (1H, m), 5.28-5.38 (1H, m), 7.72 (1H, d), 8.06 (1H, d), 8.68 (1H, s), 8.74 (1H, s). Mass Spectrum: m/z (ES+)[M+H]+=376.

### Intermediate D2: 8-Bromo-1-(oxan-4-yl)-3H-imidazo[4,5-c]quinolin-2-one

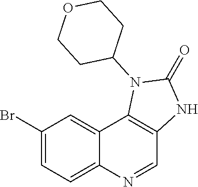 [SMILES: O=c1[nH]c2cnc3ccc(Br)cc3c2n1C1CCOCC1]

Triethylamine (143 mL, 1025.07 mmol) was added to 6-bromo-4-(oxan-4-ylamino)quinoline-3-carboxylic acid (120 g, 341.69 mmol) in DMF (600 mL) at ambient temperature under air. The resulting mixture was stirred for 30 minutes then diphenyl phosphorazidate (113 g, 410.03 mmol) was added. The resulting mixture was stirred for 30 minutes at ambient temperature then at 60° C. for 2 h. The solvent was removed under reduced pressure and the reaction mixture diluted with water. The precipitate was collected by filtration, washed with water (250 mL) and dried under vacuum to afford the desired material (120 g, 101%) as a brown solid, which was used without further purification. NMR Spectrum: ¹H NMR (400 MHz, DMSO-d6) δ 1.72-1.95 (2H, m), 2.59-2.80 (2H, m), 3.58 (2H, td), 3.98-4.11 (2H, m), 4.75-5.04 (1H, m), 7.75 (1H, dd), 7.97 (1H, d), 8.43 (1H, s), 8.71 (1H, s), 11.71 (1H, s). Mass Spectrum: m/z (ES+)[M+H]+=348.

On a larger scale, 6-bromo-4-(oxan-4-ylamino)quinoline-3-carboxylic acid (2011 g, (2005 g active), 5.71 mol) was added to the vessel with DMF (18.2 L). Triethylamine (4.7 L, 33.72 mol) was added with an endotherm observed from 21-18° C. Diphenyl phosphorazidate (1600 mL, 7.42 mol) was added over 10 minutes with an observed exotherm from 21° C. to 23° C. over the addition. The exotherm continued with the batch reaching 55° C. after 1 h (jacket held at 30° C.) with gas evolution. The reaction initially went into solution with a precipitate then forming after −30 minutes. Once the temperature had stabilised the batch was analysed by HPLC showing consumption of starting material and 99% product. The batch was heated to 60° C. for h with HPLC again indicating consumption of starting material and 98% product. The batch was concentrated in vacuo to a minimum volume (˜3 volumes) and the residue added to water (17 L) rinsing in with a further portion of water (10 L). The mixture was slurried for 1 h and filtered, washing with water (2×17 L). The solid was then returned to the vessel and slurried in sat. NaHCO₃ solution (10 L) and MeOH (495 mL) for 1 h. The solid was collected by filtration, washing with water (2×3.5 L) and then oven dried in vacuo at 40° C. for 116 h to obtain 2023 g of desired material. Analytical data was consistent with that obtained from previous batches.

The following 3H-imidazo[4,5-c]quinolin-2-one intermediates were prepared in a similar fashion from the appropriate carboxylic acid intermediates:

| Inter- | | |
|---|---|---|
| medi- | | |
| ate | Structure | Name |
| Inter- medi- ate E2 | 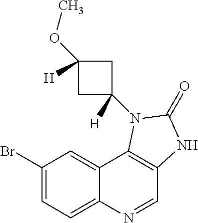 [SMILES: CO[C@H]1C[C@@H](n2c(=O)[nH]c3cnc4ccc(Br)cc4c32)C1] | 8-Bromo-1-(cis-3- methoxycyclobutyl)- 3H-imidazo[4,5- c]quinolin-2-one |
| Inter- medi- ate F2 | 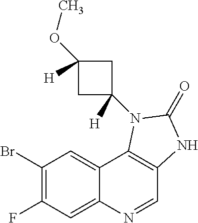 [SMILES: CO[C@H]1C[C@@H](n2c(=O)[nH]c3cnc4cc(F)c(Br)cc4c32)C1] | 8-Bromo-7-fluoro-1- (cis-3- methoxycyclobutyl)- 3H-imidazo[4,5- c]quinolin-2-one |
| Inter- medi- ate G2* | 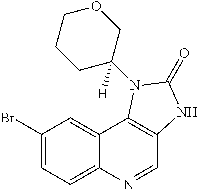 [SMILES: O=c1[nH]c2cnc3ccc(Br)cc3c2n1[C@H]1CCCOC1] | 8-bromo-1-[(3S)- oxan-3-yl]-3H- imidazo[4,5- c]quinolin-2-one |
| Inter- medi- ate H2* | 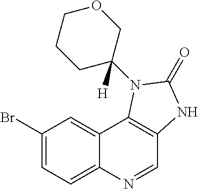 [SMILES: O=c1[nH]c2cnc3ccc(Br)cc3c2n1[C@@H]1CCCOC1] | 8-bromo-1-[(3R)- oxan-3-yl]-3H- imidazo[4,5- c]quinolin-2-one |
| Inter- medi- ate I2 | 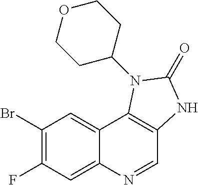 [SMILES: O=c1[nH]c2cnc3cc(F)c(Br)cc3c2n1C1CCOCC1] | 8-bromo-7-fluoro-1- (oxan-4-yl)-3H- imidazo[4,5- c]quinolin-2-one |
| Inter- medi- ate J2* | 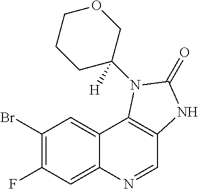 [SMILES: O=c1[nH]c2cnc3cc(F)c(Br)cc3c2n1[C@H]1CCCOC1] | 8-bromo-7-fluoro-1- [(3S)-oxan-3-yl]-3H- imidazo[4,5- c]quinolin-2-one |
| Inter- medi- ate K2* | 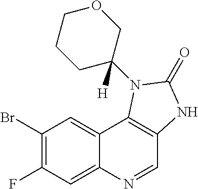 [SMILES: O=c1[nH]c2cnc3cc(F)c(Br)cc3c2n1[C@@H]1CCCOC1] | 8-bromo-7-fluoro-1- [(3R)-oxan-3-yl]-3H- imidazo[4,5- c]quinolin-2-one |
| Inter- medi- ate L2** | 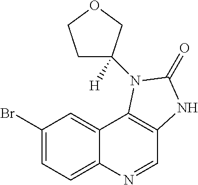 [SMILES: O=c1[nH]c2cnc3ccc(Br)cc3c2n1[C@H]1CCOC1] | 8-bromo-1-[(3S)- tetrahydrofuran-3- yl]-3H-imidazo[4,5- c]quinolin-2-one |
| Inter- medi- ate M2** | 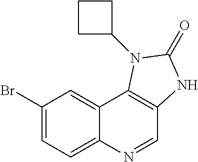 [SMILES: O=c1[nH]c2cnc3ccc(Br)cc3c2n1C1CCC1] | 8-bromo-1- cyclobutyl-3H- imidazo[4,5- c]quinolin-2-one |
| Inter- medi- ate N2* | 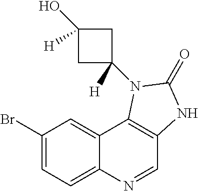 [SMILES: O=c1[nH]c2cnc3ccc(Br)cc3c2n1[C@H]1C[C@H](O)C1] | 8-bromo-1-(trans-3- hydroxycyclobutyl)- 3H-imidazo[4,5- c]quinolin-2-one |
| Inter- medi- ate O2* | 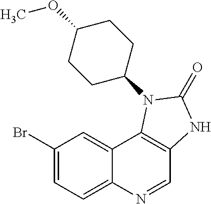 [SMILES: CO[C@H]1CC[C@H](n2c(=O)[nH]c3cnc4ccc(Br)cc4c32)CC1] | 8-bromo-1-(trans-4- methoxycyclohexyl)- 3H-imidazo[4,5- c]quinolin-2-one |
| Inter- medi- ate P2* | 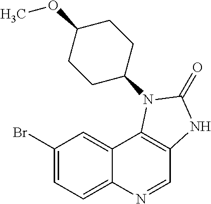 [SMILES: CO[C@H]1CC[C@@H](n2c(=O)[nH]c3cnc4ccc(Br)cc4c32)CC1] | 8-bromo-1-(cis-4- methoxycyclohexyl)- 3H-imidazo[4,5- c]quinolin-2-one |
| Inter- medi- ate Q2** | 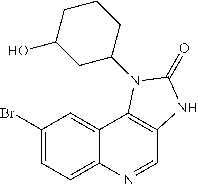 [SMILES: O=c1[nH]c2cnc3ccc(Br)cc3c2n1C1CCCC(O)C1] | 8-bromo-1-(3- hydroxycyclohexyl)- 3H-imidazo[4,5- c]quinolin-2-one (mixture of isomers) |
| Inter- medi- ate U2** | 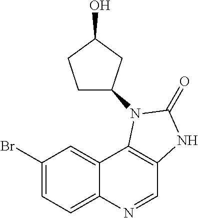 [SMILES: O=c1[nH]c2cnc3ccc(Br)cc3c2n1[C@H]1CC[C@@H](O)C1] | 8-bromo-1-[cis-3- hydroxycyclopentyl]- 3H-imidazo[4,5- c]quinolin-2-one (1:1 mixture of isomers) |

*The reaction was stirred at 60° C. for 60-90 mins.
**The reaction was stirred at 60° C. overnight.

Intermediate E2:

NMR Spectrum: ¹H NMR (400 MHz, DMSO-d6) δ 2.75-2.82 (2H, m), 2.9-3.05 (2H, m), 3.22 (3H, s), 3.80-3.90 (1H, m), 4.85-4.99 (1H, m), 7.71 (1H, dd), 7.94 (1H, d), 8.48 (1H, d), 8.69 (1H, s), 10.42 (1H, s). Mass Spectrum: m/z (ES+)[M+H]+=348, 350.

Intermediate F2:

NMR Spectrum: ¹H NMR (300 MHz, CDCl₃) δ 2.75 (2H, m), 2.95 (2H, m), 3.25 (3H, s), 3.85 (1H, m), 4.75 (1H, m), 8.00 (1H, d), 8.62-8.58 (2H, t). Mass Spectrum: m/z (ES+)[M+H]+=366.

Intermediate G2:

NMR Spectrum: ¹H NMR (300 MHz, DMSO-d6) δ 1.84-2.11 (3H, m), 2.62-2.76 (1H, m), 3.35-3.44 (1H, m), 3.92-4.22 (3H, m), 4.71-4.80 (1H, m), 7.76 (1H, dd), 7.98 (2H, d), 8.32 (1H, dd), 8.71 (1H, s), 11.85 (1H, bs). Mass Spectrum: m/z (ES+)[M+H]+=350.

Intermediate H2:

NMR Spectrum: ¹H NMR (300 MHz, DMSO-d6) δ 1.82-2.11 (3H, m), 2.61-2.75 (1H, m), 3.34-3.43 (1H, m), 3.91-4.21 (3H, m), 4.69-4.78 (1H, m), 7.75 (1H, dd), 7.99 (2H, d), 8.33 (1H, dd), 8.69 (1H, s), 11.70 (1H, bs). Mass Spectrum: m/z (ES+)[M+H]+=350.

Intermediate I2:

NMR Spectrum: ¹H NMR (400 MHz, DMSO-d6, 100° C.) δ 1.88 (2H, dd), 2.71 (2H, qd), 3.59 (2H, td), 4.06 (2H, dd), 4.92 (1H, tt), 7.92 (1H, d), 8.57 (1H, d), 8.72 (1H, s), 11.43 (1H, s). Mass Spectrum: m/z (ES+)[M+H]+=367.92.

Intermediate J2:

NMR Spectrum: ¹H NMR (400 MHz, DMSO-d6) δ 1.77-1.93 (2H, m), 2.10 (1H, d), 2.68 (1H, qd), 3.34-3.44 (1H, m), 3.94 (1H, d), 4.08 (1H, dd), 4.18 (1H, t), 4.75 (1H, ddd), 7.94 (1H, d), 8.48 (1H, d), 8.69 (1H, s), 11.63 (1H, s). Mass Spectrum: m/z (ES+)[M+H]+=366, 368.

Intermediate K2:

NMR Spectrum: ¹H NMR (400 MHz, DMSO-d6) δ 1.7-1.93 (2H, m), 2.10 (1H, d), 2.63-2.75 (1H, m), 3.49-3.61 (1H, m), 3.84-4.03 (1H, m), 4.08 (1H, dd), 4.19 (1H, t), 4.76 (1H, t), 7.95 (1H, d), 8.49 (1H, d), 8.70 (1H, s), 11.66 (1H, s). Mass Spectrum: m/z (ES+)[M+H]+=366, 368.

Intermediate L2:

Mass Spectrum: m/z (ES+)[M+H]+=334.

Intermediate M2:

Mass Spectrum: m/z (ES+)[M+H]+=318.

Intermediate N2:

NMR Spectrum: ¹H NMR (500 MHz, DMSO-d6) δ 2.32-2.44 (2H, m), 3.18-3.28 (2H, m), 4.45 (1H, d), 5.26 (1H, d), 5.42 (1H, ddd), 7.71 (1H, dd), 7.93 (1H, d), 8.29 (1H, d), 8.65 (1H, s), 11.56 (1H, s). Mass Spectrum: m/z (ES+)[M+H]+=334, 336.

Intermediate O2:

NMR Spectrum: ¹H NMR (300 MHz, DMSO-d6) δ 1.41 (2H, q), 1.96 (2H, d), 2.17 (2H, d), 2.49 (2H, d), 3.23 (1H, d), 3.32 (2H, s), 4.65 (1H, t), 7.73 (1H, dd), 7.95 (1H, d), 8.32 (1H, d), 8.66 (1H, s), 11.58 (1H, s). Mass Spectrum: m/z (ES+)[M+H]+=376.

Intermediate P2:

NMR Spectrum: ¹H NMR (400 MHz, CDCl₃) δ 1.73 (4H, dd), 2.30 (2H, d), 2.69 (2H, s), 3.59 (3H, s), 3.69 (1H, s), 4.99 (1H, s), 7.74 (1H, dd), 8.05 (1H, d), 8.88 (1H, s), 10.39 (1H, s). Mass Spectrum: m/z (ES+)[M+H]+=376.

Intermediate Q2:

Mixture of cis and trans isomers (ratio 1:2, unassigned) NMR Spectrum: ¹H NMR (400 MHz, DMSO-d6) δ 1.09-1.34 (2H, m), 1.35-1.58 (2H, m), 1.58-1.79 (1H, m), 1.78-2.07 (6H, m), 2.07-2.47 (4H, m), 3.01-3.15 (1H, m), 3.51-3.73 (1H, m), 4.19 (1H, s), 4.53-4.77 (1H, m), 4.8-4.96 (2H, m), 5.03 (1H, s), 7.74 (2H, 2×d), 7.97 (2H, 2×d), 8.31 (1H, s), 8.55 (1H, s), 8.66 (1H, s), 8.68 (1H, s), 11.56 (1H, s), 11.62 (1H, s). Mass Spectrum: m/z (ES+)[M+H]+=362.

Intermediate U2:

NMR Spectrum: ¹H NMR (400 MHz, DMSO-d6) δ 1.86-1.91 (2H, m), 1.99-2.09 (1H, m), 2.15-2.12 (1H, m), 2.33-2.46 (2H, m), 4.23-4.27 (1H, m), 5.15 (1H, d), 5.24-5.33 (1H, m), 7.74 (1H, dd), 7.96 (1H, d), 8.65 (1H, d), 8.71 (1H, s), 11.79 (1H, s). Mass Spectrum: m/z (ES+)[M+H]+=348.

### Intermediate D3: 6-Bromo-4-(oxan-4-ylamino)quinoline-3-carboxylic acid

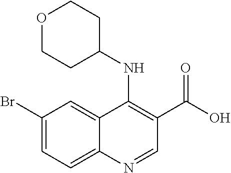 [SMILES: O=C(O)c1cnc2ccc(Br)cc2c1NC1CCOCC1]

A solution of sodium hydroxide (79 g, 1977.60 mmol) in water (1500 mL) was added to a stirred mixture of ethyl 6-bromo-4-(oxan-4-ylamino)quinoline-3-carboxylate (150 g, 395.52 mmol) in MeOH (1500 mL) at ambient temperature under air. The resulting mixture was stirred at 70° C. for 2 h then the solvent removed under reduced pressure. The reaction mixture was adjusted to pH=3 with 2M hydrochloric acid. The precipitate was collected by filtration, washed with water (500 mL) and dried under vacuum to afford the desired material (120 g, 86%) as a white solid, which was used without further purification. NMR Spectrum: ¹H NMR (400 MHz, DMSO-d6) δ 1.75-1.82 (2H, m), 2.05-2.09 (2H, m), 3.85-3.94 (5H, m), 7.95 (1H, d), 8.18 (1H, d), 8.65 (1H, s), 9.01 (1H, s). Mass Spectrum: m/z (ES+)[M+H]+=351.1.

On a larger scale, ethyl 6-bromo-4-(oxan-4-ylamino)quinoline-3-carboxylate (1925 g, 5.08 mol) was charged to the vessel with EtOH (12.5 L). 2M NaOH (12.5 L, 25.03 mol) was then added with an exotherm from 22-35° C. over the 20 minute addition. The batch was heated to 70-80° C. for 17 h at which point HPLC indicated 98.3% product and <1% starting material. The batch was concentrated in vacuo to remove EtOH and returned to the vessel. A 2M HCl solution (13 L) was then added until pH 5-6 was obtained maintaining a batch temperature below 50° C. An exotherm from 20-32° C. was observed over the 40 minute addition. A precipitate formed which was slurried at 20-25° C. for 1.5 h before filtration, washing with water until pH neutral (3×7 L). The collected solid was dried to under vacuum at 70° C. to give 1794 g of desired material. Analytical data was consistent with that obtained from previous batches.

The following carboxylic acid intermediates were prepared in a similar fashion from the appropriate ester precursor:

| Intermediate | Structure | Name |
|---|---|---|
| Intermediate E3* | 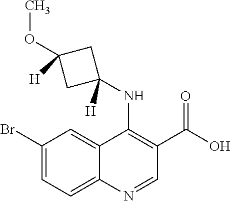 [SMILES: CO[C@H]1C[C@@H](Nc2c(C(=O)O)cnc3ccc(Br)cc23)C1] | 6-Bromo-4-[(cis-3- methoxycyclobutyl)amino]quinoline- 3-carboxylic acid |
| Intermediate F3 | 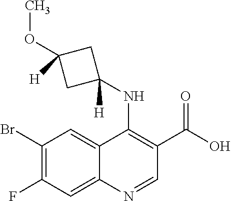 [SMILES: CO[C@H]1C[C@@H](Nc2c(C(=O)O)cnc3cc(F)c(Br)cc23)C1] | 6-Bromo-7-fluoro-4-[(cis-3- methoxycyclobutyl)amino]quinoline- 3-carboxylic acid |
| Intermediate G3** | 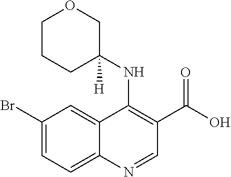 [SMILES: O=C(O)c1cnc2ccc(Br)cc2c1N[C@H]1CCCOC1] | 6-bromo-4-[[(3S)-oxan-3- yl]amino]quinoline-3-carboxylic acid |
| Intermediate H3** | 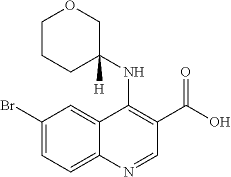 [SMILES: O=C(O)c1cnc2ccc(Br)cc2c1N[C@@H]1CCCOC1] | 6-bromo-4-[[(3R)-oxan-3- yl]amino]quinoline-3-carboxylic acid |
| Intermediate I3*** | 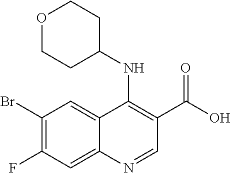 [SMILES: O=C(O)c1cnc2cc(F)c(Br)cc2c1NC1CCOCC1] | 6-bromo-7-fluoro-4-(oxan-4- ylamino)quinoline-3-carboxylic acid |
| Intermediate J3**** | 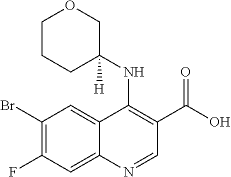 [SMILES: O=C(O)c1cnc2cc(F)c(Br)cc2c1N[C@H]1CCCOC1] | 6-bromo-7-fluoro-4-[[(3S)- tetrahydropyran-3- yl]amino]quinoline-3-carboxylic acid |
| Intermediate K3**** | 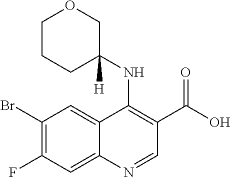 [SMILES: O=C(O)c1cnc2cc(F)c(Br)cc2c1N[C@@H]1CCCOC1] | 6-bromo-7-fluoro-4-[[(3R)- tetrahydropyran-3- yl]amino]quinoline-3-carboxylic acid |
| Intermediate L3**** | 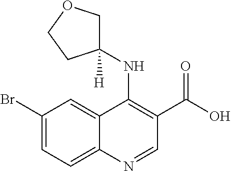 [SMILES: O=C(O)c1cnc2ccc(Br)cc2c1N[C@H]1CCOC1] | 6-bromo-4-[[(3S)-tetrahydrofuran-3- yl]amino]quinoline-3-carboxylic acid |
| Intermediate M3**** | 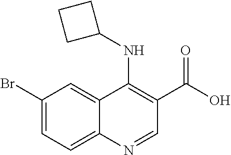 [SMILES: O=C(O)c1cnc2ccc(Br)cc2c1NC1CCC1] | 6-bromo-4- (cyclobutylamino)quinoline-3- carboxylic acid |
| Intermediate N3**** | 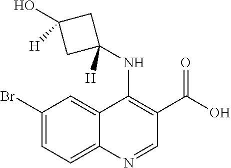 [SMILES: O=C(O)c1cnc2ccc(Br)cc2c1N[C@H]1C[C@H](O)C1] | 6-bromo-4-[(trans-3- hydroxycyclobutyl)amino]quinoline- 3-carboxylic acid |
| Intermediate O3**** | 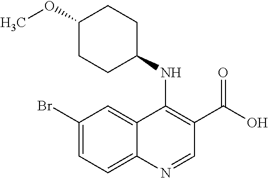 [SMILES: CO[C@H]1CC[C@H](Nc2c(C(=O)O)cnc3ccc(Br)cc23)CC1] | 6-bromo-4-[(trans-4- methoxycyclohexyl)amino]quinoline- 3-carboxylic acid |
| Intermediate P3**** | 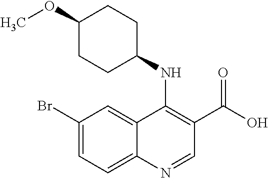 [SMILES: CO[C@H]1CC[C@@H](Nc2c(C(=O)O)cnc3ccc(Br)cc23)CC1] | 6-bromo-4-[(cis-4- methoxycyclohexyl)amino]quinoline- 3-carboxylic acid |
| Intermediate Q3**** | 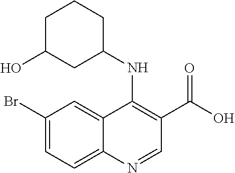 [SMILES: O=C(O)c1cnc2ccc(Br)cc2c1NC1CCCC(O)C1] | 6-bromo-4-[(3- hydroxycyclohexyl)amino]quinoline- 3-carboxylic acid (mixture of isomers) |
| Intermediate U3**** | 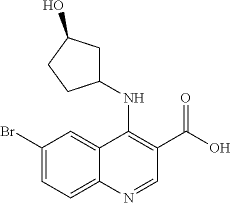 [SMILES: O=C(O)c1cnc2ccc(Br)cc2c1NC1CC[C@@H](O)C1] | 6-bromo-4-[[cis-3- hydroxycyclopentyl]amino]quinoline- 3-carboxylic acid (1:1 mixture of isomers) |

*The reaction was performed using a mixture of THF, MeOH and water as the solvent.
**The reaction was stirred between 60-70° C. for 1-3 h.
***The reaction was stirred at ambient temperature overnight.
****The reaction was performed using a mixture of THF and water as the solvent and heated at 60° C. for 3-16 h.

Intermediate E3:

Mass Spectrum: m/z (ES+)[M+H]+=351

Intermediate F3:

NMR Spectrum: ¹H NMR (400 MHz, DMSO-d6) δ 1.98-1.91 (2H, m), 2.88-2.84 (2H, m), 3.17 (1H, s), 3.77-3.70 (1H, t), 4.22-4.19 (1H, t), 7.73 (1H, d), 8.44 (1H, d), 8.88 (1H, s), 13.27 (1H, s). Mass Spectrum: m/z (ES+)[M+H]+=369.

Intermediate G3:

NMR Spectrum: ¹H NMR (300 MHz, DMSO-d6) δ 1.50-1.57 (1H, m), 1.61-1.82 (2H, m), 1.98-2.13 (1H, m), 3.48-3.72 (3H, m), 3.89 (1H, d), 4.15-4.26 (1H, m), 7.77 (1H, dd), 7.95 (1H, d), 8.31 (1H, d), 8.90 (1H, s), 13.38 (1H, bs). Mass Spectrum: m/z (ES+)[M+H]+=351.

Intermediate H3:

NMR Spectrum: ¹H NMR (300 MHz, DMSO-d6) δ 1.50-1.56 (1H, m), 1.62-1.83 (2H, m), 1.99-2.12 (1H, m), 3.50-3.71 (3H, m), 3.89 (1H, d), 4.16-4.28 (1H, m), 7.78 (1H, dd), 7.94 (1H, d), 8.30 (1H, d), 8.94 (1H, s), 13.50 (1H, bs). Mass Spectrum: m/z (ES+)[M+H]+=351.

Intermediate I3:

Mass Spectrum: m/z (ES+)[M+H]+=369.

Intermediate J3:

NMR Spectrum: ¹H NMR (300 MHz, DMSO-d6) δ 1.51 (1H, m), 1.74 (2H, m), 2.04 (1H, m), 3.60 (3H, m), 3.82 (1H, d), 4.15 (1H, m), 7.73 (1H, m), 8.44 (1H, m), 8.92 (1H, s). Mass Spectrum: m/z (ES+)[M+H]+=369.

Intermediate K3:

Mass Spectrum: m/z (ES+)[M+H]+=369.

Intermediate L3:

NMR Spectrum: ¹H NMR (400 MHz, DMSO-d6) δ 1.95-2.05 (1H, m), 2.31-2.41 (1H, m), 3.79-3.87 (2H, m), 3.89-3.95 (2H, m), 4.82-4.92 (1H, m), 7.78 (1H, d), 7.92-7.94 (1H, m), 8.44 (1H, d), 8.90 (1H, s), 13.3 (1H, s). Mass Spectrum: m/z (ES+)[M+H]+=337.

Intermediate M3:

NMR Spectrum: ¹H NMR (400 MHz, DMSO-d6) δ 1.81-1.95 (3H, m), 2.01-2.15 (3H, m), 4.53-4.55 (1H, m), 7.74 (1H, d), 7.88 (1H, d), 8.25 (1H, s), 8.89 (1H, s), 13.27 (1H, s). Mass Spectrum: m/z (ES+)[M+H]+=321.

Intermediate N3:

NMR Spectrum: ¹H NMR (500 MHz, DMSO-d6) δ 2.27-2.46 (4H, m), 4.36 (1H, s), 4.71 (1H, d), 5.28 (1H, s), 7.75 (1H, d), 7.92 (1H, dd), 8.22 (1H, dd), 8.85 (1H, s). Mass Spectrum: m/z (ES+)[M+H]+=337.

Intermediate O3:

Mass Spectrum: m/z (ES+)[M+H]+=379.

Intermediate P3:

NMR Spectrum: ¹H NMR (400 MHz, DMSO-d6) δ 1.66 (2H, s), 1.84 (6H, s), 3.27 (3H, s), 3.41 (1H, s), 7.96 (1H, d), 8.19 (1H, d), 9.02 (1H, s). Mass Spectrum: m/z (ES+)[M+H]+=379.

Intermediate Q3:

Mixture of cis and trans isomers (ratio 1:2, unassigned) NMR Spectrum: ¹H NMR (400 MHz, DMSO-d6) δ 1.09-1.25 (2H, m), 1.26-1.46 (4H, m), 1.48-1.66 (2H, m), 1.68-1.92 (4H, m), 1.92-2.10 (3H, m), 2.27 (1H, d), 3.49-3.64 (2H, m), 3.99 (1H, s), 4.10 (2H, s), 4.51 (1H, s), 4.72 (1H, s), 4.83 (1H, s), 7.84 (2H, 2×d), 8.01 (2H, 2×d), 8.42 (1H, s), 8.48 (1H, s), 8.91 (2H, 2×s). Mass Spectrum: m/z (ES+)[M+H]+=365.

Intermediate U3:

NMR Spectrum: ¹H NMR (300 MHz, DMSO-d6) δ 1.70-1.81 (3H, m), 1.89-2.00 (1H, m), 2.19-2.32 (2H, tq), 4.24 (1H, d), 4.70 (1H, t), 4.88 (1H, s), 7.87 (1H, d), 8.07 (1H, dd), 8.49 (1H, d), 8.93 (1H, s), 11.33 (1H, s). Mass Spectrum: m/z (ES+)[M+H]+=351.

### Intermediate D4: Ethyl 6-bromo-4-(oxan-4-ylamino)quinoline-3-carboxylate

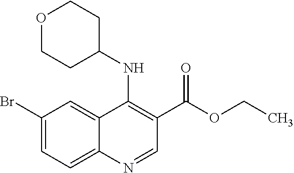 [SMILES: CCOC(=O)c1cnc2ccc(Br)cc2c1NC1CCOCC1]

DIPEA (139 mL, 794.75 mmol) was added to ethyl 6-bromo-4-chloroquinoline-3-carboxylate (100 g, 317.90 mmol) and tetrahydro-2H-pyran-4-amine (35.4 g, 349.69 mmol) in DMA (1000 mL) at ambient temperature under air. The resulting mixture was stirred at 60° C. for 16 h then the solvent removed under reduced pressure. The mixture was azeotroped twice with toluene to afford the desired material (150 g, 124%) as a brown solid, which was used without further purification. NMR Spectrum: ¹H NMR (400 MHz, DMSO-d6) δ 1.36 (3H, t), 1.58-1.75 (2H, m), 1.90-2.02 (2H, m), 3.40 (2H, t), 3.81-3.98 (2H, m), 3.98-4.19 (1H, m), 4.37 (2H, q), 7.82 (1H, d), 7.92 (1H, dd), 8.56 (1H, s), 8.86 (1H, s). Mass Spectrum: m/z (ES−)[M−H]−=378, 380.

On a larger scale, ethyl 6-bromo-4-chloroquinoline-3-carboxylate (2196 g, (1976 g active), 6.28 mol) was charged to the vessel with DMA (16 L). Tetrahydro-2H-pyran-4-amine (1224 g, 12.10 mol) was added over 10 minutes with an observed exotherm of 21-27° C. DIPEA (3.5 L, 20.09 mol) was added with no observed exotherm. The mixture was heated to 75-85° C. and the resulting solution stirred for 18.5 h at 80° C. HPLC indicated consumption of starting material and 99.2% product. The reaction was cooled to 50° C. and then poured into water (50 L). The resulting suspension was stirred for 2 h at ambient temperature and the solids isolated by filtration, washing with water (8 L then 2×4 L). The solid was dried under vacuum at 40° C. for 55 h to give 2307 g of desired material. Analytical data was consistent with that obtained from previous batches.

The following ester intermediates were prepared in an analogous fashion from the appropriate amine and either ethyl 6-bromo-4-chloro-7-fluoroquinoline-3-carboxylate or ethyl 6-bromo-4-chloroquinoline-3-carboxylate:

| Intermediate | Structure | Name |
|---|---|---|
| Intermediate E4* | 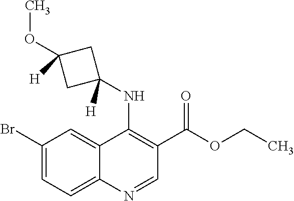 [SMILES: CCOC(=O)c1cnc2ccc(Br)cc2c1N[C@H]1C[C@@H](OC)C1] | Ethyl 6-bromo-4-[(cis-3- methoxycyclobutyl)amino]quinoline- 3-carboxylate |
| Intermediate F4** | 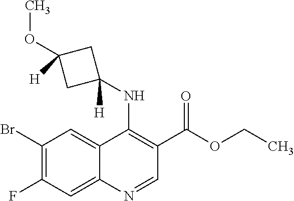 [SMILES: CCOC(=O)c1cnc2cc(F)c(Br)cc2c1N[C@H]1C[C@@H](OC)C1] | Ethyl 6-bromo-7-fluoro-4-[(cis-3- methoxycyclobutyl)amino]quinoline- 3-carboxylate |
| Intermediate G4*** | 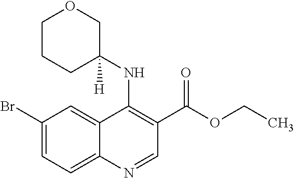 [SMILES: CCOC(=O)c1cnc2ccc(Br)cc2c1N[C@H]1CCCOC1] | ethyl 6-bromo-4-[[(3S)-oxan-3- yl]amino]quinoline-3-carboxylate |
| Intermediate H4*** | 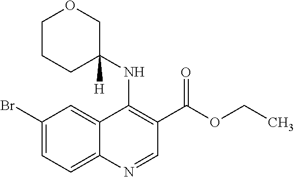 [SMILES: CCOC(=O)c1cnc2ccc(Br)cc2c1N[C@@H]1CCCOC1] | ethyl 6-bromo-4-[[(3R)-oxan-3- yl]amino]quinoline-3-carboxylate |
| Intermediate I4**** | 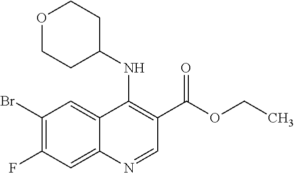 [SMILES: CCOC(=O)c1cnc2cc(F)c(Br)cc2c1NC1CCOCC1] | ethyl 6-bromo-7-fluoro-4-(oxan-4- ylamino)quinoline-3-carboxylate |
| Intermediate J4*** | 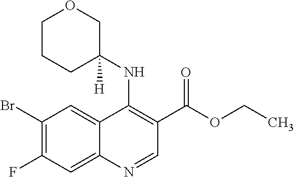 [SMILES: CCOC(=O)c1cnc2cc(F)c(Br)cc2c1N[C@H]1CCCOC1] | ethyl 6-bromo-7-fluoro-4-[[(3S)- tetrahydropyran-3- yl]amino]quinoline-3-carboxylate |
| Intermediate K4*** | 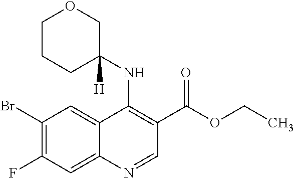 [SMILES: CCOC(=O)c1cnc2cc(F)c(Br)cc2c1N[C@@H]1CCCOC1] | ethyl 6-bromo-7-fluoro-4-[[(3R)- tetrahydropyran-3- yl]amino]quinoline-3-carboxylate |
| Intermediate L4***** | 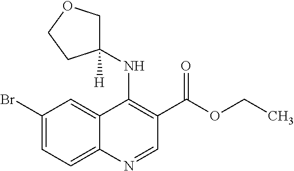 [SMILES: CCOC(=O)c1cnc2ccc(Br)cc2c1N[C@H]1CCOC1] | ethyl 6-bromo-4-[[(3S)- tetrahydrofuran-3- yl]amino]quinoline-3-carboxylate |
| Intermediate M4 | 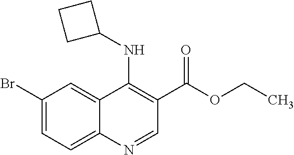 [SMILES: CCOC(=O)c1cnc2ccc(Br)cc2c1NC1CCC1] | ethyl 6-bromo-4- (cyclobutylamino)quinoline-3- carboxylate |
| Intermediate N4*** | 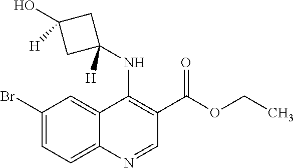 [SMILES: CCOC(=O)c1cnc2ccc(Br)cc2c1N[C@H]1C[C@H](O)C1] | ethyl 6-bromo-4-[(trans-3- hydroxycyclobutyl)amino]quinoline- 3-carboxylate |
| Intermediate O4*** | 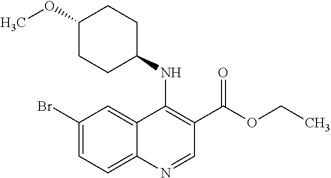 [SMILES: CCOC(=O)c1cnc2ccc(Br)cc2c1N[C@H]1CC[C@H](OC)CC1] | ethyl 6-bromo-4-[(trans-4- methoxycyclohexyl)amino]quinoline- 3-carboxylate |
| Intermediate P4*** | 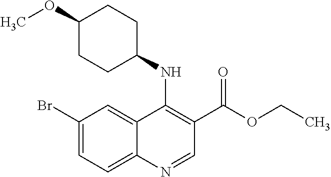 [SMILES: CCOC(=O)c1cnc2ccc(Br)cc2c1N[C@H]1CC[C@@H](OC)CC1] | ethyl 6-bromo-4-[(cis-4- methoxycyclohexyl)amino]quinoline- 3-carboxylate |
| Intermediate Q4*** | 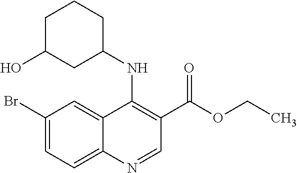 [SMILES: CCOC(=O)c1cnc2ccc(Br)cc2c1NC1CCCC(O)C1] | ethyl 6-bromo-4-[(3- hydroxycyclohexyl)amino]quinoline- 3-carboxylate (mixture of isomers) |
| Intermediate U3*** | 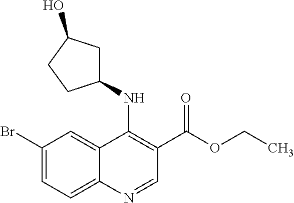 [SMILES: CCOC(=O)c1cnc2ccc(Br)cc2c1N[C@H]1CC[C@@H](O)C1] | ethyl 6-bromo-4-[[(1S,3R)-3- hydroxycyclopentyl]amino]quinoline- 3-carboxylate (1:1 mixture of isomers) |

*The reaction was stirred at 75° C. for 5 h.
**The reaction was stirred at 85° C. for 3 h.
***The reaction was stirred at 80° C. for 2-16 h.
****The reaction was stirred at 90° C. for 1-3 h.
*****The reaction was stirred at 100° C. for 16 h.

Intermediate E4:

NMR Spectrum: ¹H NMR (300 MHz, DMSO-d6) δ 1.38 (3H, t), 1.85-1.98 (2H, m), 2.75-7.89 (2H, m), 3.17 (3H, s), 3.65-3.78 (1H, m), 3.98-4.05 (1H, m), 4.35 (2H, q), 7.60 (1H, d), 7.70 (1H, dd), 8.40 (1H, d), 8.84-8.85 (1H, m). Mass Spectrum: m/z (ES+)[M+H]+=379.

Intermediate F4:

NMR Spectrum: ¹H NMR (400 MHz, CDCl₃) δ 1.44-1.41 (3H, t), 2.21-2.14 (2H, m), 3.05-2.98 (2H, m), 3.30 (3H, s), 3.94-3.75 (1H, m), 4.11-4.06 (1H, m), 4.43-4.37 (2H, d), 7.70 (1H, d), 8.29 (1H, d), 9.07 (1H, d), 9.69 (1H, s). Mass Spectrum: m/z (ES+)[M+H]+=397.

Intermediate G4:

NMR Spectrum: ¹H NMR (300 MHz, DMSO-d6) δ 1.36 (3H, t), 1.70-1.74 (1H, m), 1.75-1.77 (2H, m), 2.03-2.05 (1H, m), 3.58-3.61 (3H, m), 3.80-3.85 (1H, m), 4.01-4.03 (1H, m), 4.35 (2H, q), 7.80 (1H, d), 7.89 (1H, dd), 8.58 (1H, s), 8.67 (1H, d), 8.93 (1H, s). Mass Spectrum: m/z (ES+)[M+H]+=380.8.

Intermediate H4:

NMR Spectrum: ¹H NMR (400 MHz, DMSO-d6) δ 1.50-1.56 (1H, m), 1.62-1.84 (2H, m), 1.99-2.13 (1H, m), 3.51-3.73 (3H, m), 3.89 (1H, d), 4.12-4.22 (1H, m), 7.77 (1H, d), 7.90 (1H, d), 8.31 (1H, s), 8.94 (1H, s), 13.41 (1H, bs). Mass Spectrum: m/z (ES+)[M+H]+=379.

Intermediate I4:

Mass Spectrum: m/z (ES+)[M+H]+=397.

Intermediate J4:

NMR Spectrum: ¹H NMR (300 MHz, DMSO-d6) δ 1.33 (3H, m), 1.51 (1H, m), 1.74 (2H, m), 2.04 (1H, m), 3.60 (3H, m), 3.82 (1H, d), 4.02 (1H, m), 4.35 (2H, m), 7.73 (1H, m), 8.49 (1H, m), 8.79 (1H, m), 8.88 (1H, s). Mass Spectrum: m/z (ES+)[M+H]+=397.

Intermediate K4:

Mass Spectrum: m/z (ES+)[M+H]+=397.

Intermediate L4:

NMR Spectrum: ¹H NMR (400 MHz, CDCl₃) δ 1.45 (3H, t), 2.12-2.19 (1H, m), 2.48-2.55 (1H, m), 3.87-4.04 (2H, m), 4.12 (2H, td), 4.43 (2H, q), 4.76-4.86 (1H, m), 7.80 (1H, dd), 7.95 (1H, d), 8.34 (1H, d), 9.14 (1H, s), 9.64 (1H, s). Mass Spectrum: m/z (ES+)[M+H]+=365.

Intermediate M4:

NMR Spectrum: ¹H NMR (400 MHz, CDCl₃) δ 1.45 (3H, t), 1.77-2.01 (2H, m), 2.16-2.31 (2H, m), 2.58-2.71 (2H, m), 4.45 (3H, m), 7.74 (1H, dd), 7.82 (1H, d), 8.23 (1H, d), 9.09 (1H, s), 9.57 (1H, d) Mass Spectrum: m/z (ES+)[M+H]+=349.

Intermediate N4:

NMR Spectrum: ¹H NMR (500 MHz, DMSO-d6) δ 1.34 (3H, t), 2.34 (4H, t), 4.33 (3H, q), 4.56 (1H, q), 5.21 (1H, d), 7.75 (1H, d), 7.85 (1H, dd), 8.31 (1H, d), 8.85 (1H, s), 9.13 (1H, d). Mass Spectrum: m/z (ES+)[M+H]+=366.

Intermediate O4:

NMR Spectrum: ¹H NMR (400 MHz, CDCl₃) δ 1.40-1.59 (1H, 4H), 1.45 (3H, t), 2.08-2.18 (2H, m), 2.18-2.27 (2H, m), 3.23-3.34 (1H, m), 3.39 (3H, s), 3.99-4.05 (1H, m), 4.41 (2H, q), 7.75 (1H, dd), 7.83 (1H, d), 8.27 (1H, d), 9.08 (1H, d), 9.12 (1H, s) Mass Spectrum: m/z (ES+)[M+H]+=407.

Intermediate P4:

NMR Spectrum: ¹H NMR (400 MHz, DMSO-d6) δ 1.35 (3H, t), 1.54-1.61 (2H, m), 1.63-1.83 (6H, m), 3.24 (3H, s), 3.96 (1H, d), 4.35 (2H, q), 7.78 (1H, d), 7.87 (1H, dd), 8.44 (1H, d), 8.61 (1H, d), 8.87 (1H, s). Mass Spectrum: m/z (ES+)[M+H]+=407.

Intermediate Q4:

Mixture of cis and trans isomers (ratio 1:2, unassigned) NMR Spectrum: ¹H NMR (400 MHz, DMSO-d6) δ 1.06-1.2 (2H, m), 1.21-1.42 (10H, m), 1.42-1.61 (2H, m), 1.63-1.86 (4H, m), 1.87-2.01 (2H, m), 2.20 (1H, d), 3.39-3.57 (2H, m), 3.71-3.87 (1H, m), 3.95 (1H, s), 4.22-4.48 (5H, m), 4.61 (1H, s), 4.79 (1H, s), 7.77 (1H, s), 7.80 (1H, s), 7.84-7.90 (2H, m), 8.35 (1H, d), 8.42 (2H, 2×d), 8.69 (1H, d), 8.84 (1H, s), 8.88 (1H, s). Mass Spectrum: m/z (ES+)[M+H]+=393.

Intermediate U4:

NMR Spectrum: ¹H NMR (300 MHz, CDCl₃) δ 1.42 (3H, t), 1.85-2.05 (2H, m), 2.05-2.22 (1H, m), 2.29-2.41 (2H, m), 4.39 (2H, q), 4.52-4.62 (2H, m), 7.72 (1H, dd), 7.82 (1H, d), 8.35 (1H, d), 9.08 (1H, s), 9.58 (1H, d). Mass Spectrum: m/z (ES+)[M+H]+=379.

The preparation of 8-bromo-1-[(1R,3R)-3-methoxycyclopentyl]-3-methylimidazo[4,5-c]quinolin-2-one: 8-bromo-1-[(1S,3S)-3-methoxycyclopentyl]-3-methylimidazo[4,5-c]quinolin-2-one (1:1 mixture) is described below:

### Intermediate VI: 8-bromo-1-[(1R,3R)-3-methoxycyclopentyl]-3-methylimidazo[4,5-c]quinolin-2-one: 8-bromo-1-[(1S,3S)-3-methoxycyclopentyl]-3-methylimidazo[4,5-c]quinolin-2-one (1:1 Mixture)

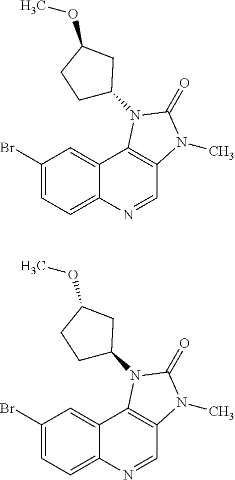 [SMILES: CO[C@@H]1CC[C@@H](n2c(=O)n(C)c3cnc4ccc(Br)cc4c32)C1 ; CO[C@H]1CC[C@H](n2c(=O)n(C)c3cnc4ccc(Br)cc4c32)C1]

A mixture of 6-bromo-4-[[(1R,3R)-3-methoxycyclopentyl]amino]quinoline-3-carboxylic acid: 6-bromo-4-[[(1S,3S)-3-methoxycyclopentyl]amino]quinoline-3-carboxylic acid (1:1 mixture) (13 g, 35.8 mmol), tetrabutylammonium bromide (1.16 g, 3.60 mmol), iodomethane (7.645 g, 53.86 mmol) and sodium hydroxide (2.15 g, 53.75 mmol) in DCM (600 mL) and water (380 mL) was stirred at ambient temperature overnight. The resulting solution was concentrated under vacuum to remove the organics and the solids collected by filtration, washed with water (5×10 mL) and dried in a vacuum oven to afford the desired material (racemic mixture) (9.8 g, 73%) as a off-white solid. NMR Spectrum: ¹H NMR (400 MHz, DMSO-d6) δ 1.81-1.87 (1H, m), 2.33-2.51 (4H, m), 2.45-2.51 (1H, m), 3.28 (3H, s), 3.49 (3H, s), 4.02-4.21 (1H, m), 5.40 (1H, p), 7.73 (1H, dd), 7.98 (1H, d), 8.35 (1H, d), 8.91 (1H, s). Mass Spectrum: m/z (ES+)[M+H]+=375.9.

### Intermediate V2: 8-bromo-1-[(1R,3R)-3-methoxycyclopentyl]-3H-imidazo[4,5-c]quinolin-2-one: 8-bromo-1-[(1S,3S)-3-methoxycyclopentyl]-3H-imidazo[4,5-c]quinolin-2-one (1:1 Mixture)

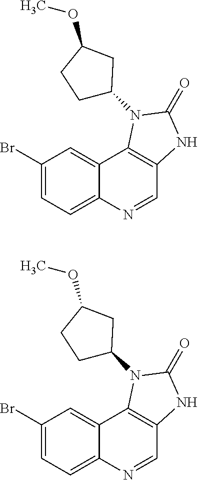 [SMILES: CO[C@@H]1CC[C@@H](n2c(=O)[nH]c3cnc4ccc(Br)cc4c32)C1 ; CO[C@H]1CC[C@H](n2c(=O)[nH]c3cnc4ccc(Br)cc4c32)C1]

A mixture of 6-bromo-4-[[(1R,3R)-3-methoxycyclopentyl]amino]quinoline-3-carboxylic acid: 6-bromo-4-[[(1S,3S)-3-methoxycyclopentyl]amino]quinoline-3-carboxylic acid (1:1 mixture) (17 g, 46.54 mmol), triethylamine (14.1 g, 139.34 mmol) in DMF (270 mL) was stirred at ambient temperature for 1 h. Diphenyl phosphorazidate (25.6 g, 93.02 mmol) was added dropwise with stirring and the solution stirred at ambient temperature for a further 20 minutes before being heated to 60° C. for 1 h. The reaction was allowed to cool and concentrated under vacuum. The residue was diluted with water (300 mL), the solids collected by filtration and dried in an oven under reduced pressure to afford the desired material (as a racemic mixture) (13 g, 77%) as a off-white solid. Mass Spectrum: m/z (ES+)[M+H]+=362.2.

### Intermediate V3: 6-bromo-4-[[(1R,3R)-3-methoxycyclopentyl]amino]quinoline-3-carboxylic acid: 6-bromo-4-[[(1S,3S)-3-methoxycyclopentyl]amino]quinoline-3-carboxylic acid (1:1 Mixture)

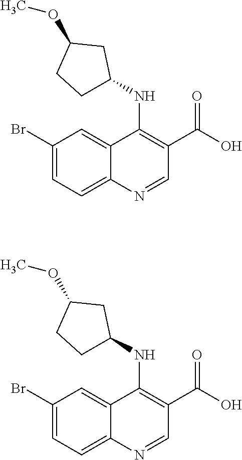 [SMILES: CO[C@@H]1CC[C@@H](Nc2c(C(=O)O)cnc3ccc(Br)cc23)C1 ; CO[C@H]1CC[C@H](Nc2c(C(=O)O)cnc3ccc(Br)cc23)C1]

2N Sodium hydroxide (150 mL) was added to a mixture of ethyl 6-bromo-4-[[(1R,3R)-3-methoxycyclopentyl]amino]quinoline-3-carboxylate: ethyl 6-bromo-4-[[(1S,3S)-3-methoxycyclopentyl]amino]quinoline-3-carboxylate (1:1 mixture) (18.6 g, 47.2 mmol) in MeOH (500 mL) and water (100 mL) and the resulting solution stirred for 15 h at ambient temperature. The mixture was concentrated under vacuum and the residue diluted with water (300 mL). The pH value of the solution was adjusted to 5 with 2N hydrochloric acid, the solids collected by filtration and dried in an oven under reduced pressure to afford the desired material (as a racemic mixture) (17.1 g) as a off-white solid. NMR Spectrum: ¹H NMR (400 MHz, DMSO-d6) δ 1.60-1.71 (2H, m), 1.81-1.88 (1H, m), 1.96-2.02 (1H, m), 2.03-2.10 (2H, m), 3.21 (3H, s), 3.91-3.96 (1H, m), 4.51-4.72 (1H, m), 7.77 (1H, d), 7.93 (1H, d), 8.45 (1H, d), 8.85 (1H, s), 13.30 (1H, bs). Mass Spectrum: m/z (ES+)[M+H]+=365.2.

### Intermediate V4: Ethyl 6-bromo-4-[[(1R,3R)-3-methoxycyclopentyl]amino]quinoline-3-carboxylate: ethyl 6-bromo-4-[[(1S,3S)-3-methoxycyclopentyl]amino]quinoline-3-carboxylate (1:1 Mixture)

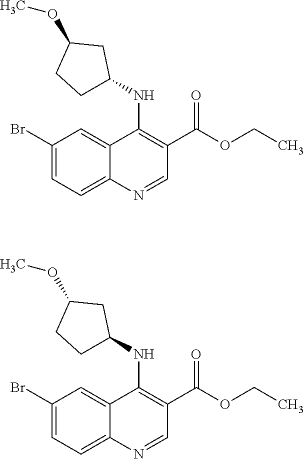

A mixture of ethyl 6-bromo-4-chloroquinoline-3-carboxylate (15 g, 47.69 mmol), (trans)-3-methoxycyclopentan-1-amine (racemic mixture) (8.09 g, 26.68 mmol) and DIPEA (19.68 g, 152.27 mmol) in DMA (100 mL) was stirred at 80° C. for 4 h under an inert atmosphere. The reaction was quenched by the addition of water (500 mL), the solids collected by filtration and dried in an oven under reduced pressure to afford the desired material (as a racemic mixture) (18.6 g) as a light brown solid. Mass Spectrum: m/z (ES+)[M+H]+=393, 395.

### Intermediate W1: 8-bromo-7-fluoro-1-[(1R,3R)-3-methoxycyclopentyl]-3-methyl-imidazo[4,5-c]quinolin-2-one and 8-bromo-7-fluoro-1-[(1S,3S)-3-methoxycyclopentyl]-3-methyl-imidazo[4,5-c]quinolin-2-one (1:1 Mixture)

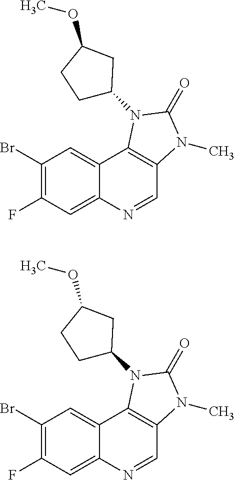 [SMILES: CO[C@@H]1CC[C@@H](n2c(=O)n(C)c3cnc4cc(F)c(Br)cc4c32)C1 ; CO[C@H]1CC[C@H](n2c(=O)n(C)c3cnc4cc(F)c(Br)cc4c32)C1]

A mixture of 8-bromo-7-fluoro-1-[(1R,3R)-3-methoxycyclopentyl]-3H-imidazo[4,5-c]quinolin-2-one: 8-bromo-7-fluoro-1-[(1S,3S)-3-methoxycyclopentyl]-3H-imidazo[4,5-c]quinolin-2-one (1:1 mixture) (2.8 g, 7.33 mmol), sodium hydroxide (440 mg, 11.00 mmol,), tetrabutylammonium bromide (240 mg, 0.75 mmol) and methyl iodide (1.6 g, 11.27 mmol) in DCM (150 mL) and water (100 mL) was stirred for 12 h at ambient temperature. The resulting mixture was concentrated in vacuo and the residue triturated with water. The solids were collected by filtration and dried to afford the desired material as a white solid (2.5 g, 86%). NMR Spectrum: ¹H NMR (300 MHz, DMSO-d6) δ 1.76-1.86 (1H, m), 2.11-2.32 (4H, m), 2.41-2.44 (1H, m), 3.27 (3H, s), 3.30 (3H, s), 4.12-4.15 (1H, m), 5.38-5.45 (1H, m), 7.96 (1H, d), 8.53 (1H, d), 8.94 (1H, s). Mass Spectrum: m/z (ES+)[M+H]+=394.

### Intermediate W2: 8-bromo-7-fluoro-1-[(1R,3R)-3-methoxycyclopentyl]-3H-imidazo[4,5-c]quinolin-2-one and 8-bromo-7-fluoro-1-[(1S,3S)-3-methoxycyclopentyl]-3H-imidazo[4,5-c]quinolin-2-one (1:1 Mixture)

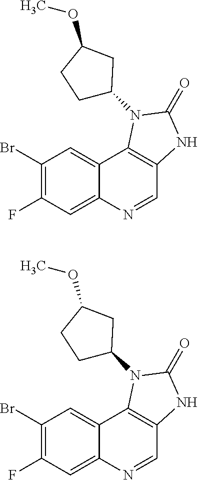 [SMILES: CO[C@@H]1CC[C@@H](n2c(=O)[nH]c3cnc4cc(F)c(Br)cc4c32)C1 ; CO[C@H]1CC[C@H](n2c(=O)[nH]c3cnc4cc(F)c(Br)cc4c32)C1]

A mixture of 6-bromo-7-fluoro-4-[[(1R,3R)-3-methoxycyclopentyl]amino]quinoline-3-carboxylic acid: 6-bromo-7-fluoro-4-[[(1S,3S)-3-methoxycyclopentyl]amino]quinoline-3-carboxylic acid (1:1 mixture) (2.9 g, 7.53 mmol) and triethylamine (2.3 g, 22.73 mmol) in DMA (20 mL) was stirred at ambient temperature for 30 mins. Diphenyl phosphorazidate (2.5 g, 9.09 mmol) was added and the resulting solution stirred for 2 h at 60° C. The reaction mixture was allowed to cool and the solids collected by filtration. The solid was dried in an oven under reduced pressure to afford the desired material as a white solid (2.8 g, 97%). NMR Spectrum: ¹H NMR (300 MHz, DMSO-d6) δ 1.78-1.88 (1H, m), 2.11-2.31 (4H, m), 2.41-2.45 (1H, m), 3.27 (3H, s), 4.08-4.15 (1H, m), 5.34-5.39 (1H, m), 7.92 (1H, d), 8.51 (1H, d), 8.68 (1H, s). Mass Spectrum: m/z (ES+)[M+H]+=380.

### Intermediate W3: 6-bromo-7-fluoro-4-[[(1R,3R)-3-methoxycyclopentyl]amino]quinoline-3-carboxylic acid and 6-bromo-7-fluoro-4-[[(1S,3S)-3-methoxycyclopentyl]amino]quinoline-3-carboxylic acid (1:1 Mixture)

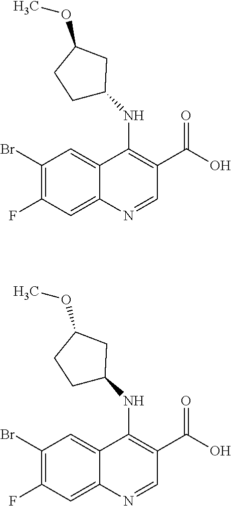 [SMILES: CO[C@@H]1CC[C@@H](Nc2c(C(=O)O)cnc3cc(F)c(Br)cc23)C1 ; CO[C@H]1CC[C@H](Nc2c(C(=O)O)cnc3cc(F)c(Br)cc23)C1]

A mixture of ethyl 6-bromo-7-fluoro-4-[[(1R,3R)-3-methoxycyclopentyl]amino]quinoline-3-carboxylate: ethyl 6-bromo-7-fluoro-4-[[(1S,3S)-3-methoxycyclopentyl]amino]quinoline-3-carboxylate (1:1 mixture) (3.4 g, 8.23 mmol) and 2N sodium hydroxide (12 mL) in MeOH (15 mL) and THF (15 mL) was stirred for 12 h at ambient temperature. The pH of the solution was adjusted to 3 with 1M HCl and the resultant solid collected by filtration and dried to afford the desired material as a white solid (2.9 g, 91%). NMR Spectrum: ¹H NMR (300 MHz, DMSO-d6) δ 1.61-1.71 (2H, m), 1.76-1.86 (1H, m), 1.92-2.03 (1H, m), 2.11-2.26 (2H, m), 3.21 (3H, s), 3.86-3.96 (1H, m), 4.56-4.64 (1H, m), 7.70 (1H, d), 8.56 (1H, d), 8.88 (1H, s), 13.31 (1H, s). Mass Spectrum: m/z (ES+)[M+H]+=383.

### Intermediate W4: Ethyl 6-bromo-7-fluoro-4-[[(1R,3R)-3-methoxycyclopentyl]amino]quinoline-3-carboxylate and Ethyl 6-bromo-7-fluoro-4-[[(1S,3S)-3-methoxycyclopentyl]amino]quinoline-3-carboxylate (1:1 Mixture)

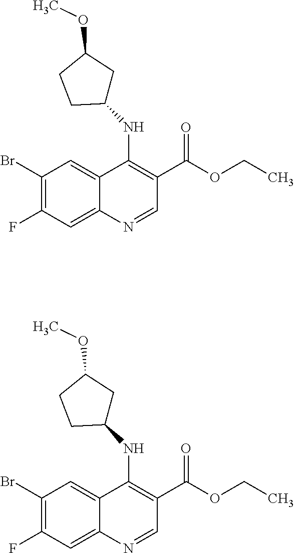 [SMILES: CCOC(=O)c1cnc2cc(F)c(Br)cc2c1N[C@@H]1CC[C@@H](OC)C1 ; CCOC(=O)c1cnc2cc(F)c(Br)cc2c1N[C@H]1CC[C@H](OC)C1]

A mixture of ethyl 6-bromo-4-chloro-7-fluoroquinoline-3-carboxylate (2 g, 6.01 mmol), (1R,3R)-3-methoxycyclopentanamine hydrochloride and (1S,3S)-3-methoxycyclopentanamine hydrochloride (1:1 mixture) (1.4 g, 9.21 mmol) and DIPEA (1.6 g, 12.38 mmol) in DMA (10 mL) was stirred for 2 h at 80° C. The reaction mixture was allowed to cool and the residue triturated with water. The solids were collected by filtration and dried to afford the desired material as a white solid (2.4 g, 97%). Mass Spectrum: m/z (ES+)[M+H]+=411.

### Intermediate X1: 8-Bromo-7-fluoro-1-[(1R,3S)-3-methoxycyclopentyl]-3-methyl-imidazo[4,5-c]quinolin-2-one and 8-bromo-7-fluoro-1-[(1S,3R)-3-methoxycyclopentyl]-3-methyl-imidazo[4,5-c]quinolin-2-one (1:1 Mixture)

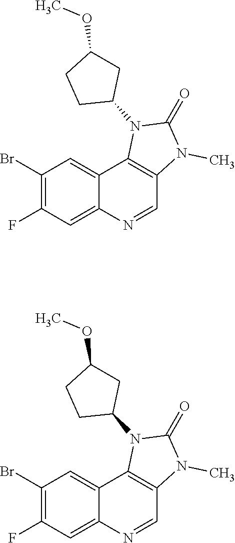 [SMILES: CO[C@H]1CC[C@@H](n2c(=O)n(C)c3cnc4cc(F)c(Br)cc4c32)C1 ; CO[C@@H]1CC[C@H](n2c(=O)n(C)c3cnc4cc(F)c(Br)cc4c32)C1]

NaH (0.213 g, 8.88 mmol) was added portionwise to 8-bromo-7-fluoro-1-[(1R,3S)-3-hydroxycyclopentyl]-3H-imidazo[4,5-c]quinolin-2-one: 8-bromo-7-fluoro-1-[(1S,3R)-3-hydroxycyclopentyl]-3H-imidazo[4,5-c]quinolin-2-one (1:1 mixture) (1.3 g, 3.55 mmol) in DMF (10 mL) at −20° C. under nitrogen and the resulting mixture stirred at 0° C. for 30 minutes. Methyl iodide (0.444 mL, 7.10 mmol) was added dropwise to the mixture at−20° C. under nitrogen and the resulting mixture was stirred at ambient temperature for 16 h.

The reaction mixture was poured into water (20 mL), the solid filtered and dried to afford the desired material as a brown solid (1.30 g, 93%). NMR Spectrum: ¹H NMR (400 MHz, DMSO-d6) δ 1.96-2.02 (3H, t), 2.22-2.51 (3H, m), 3.30-3.32 (3H, s), 3.97 (1H, m), 5.26-5.31 (1H, m), 7.89-7.52 (1H, d), 8.74 (1H, d), 8.93 (1H, s). Mass Spectrum: m/z (ES+)[M+H]+=396.

### Intermediate X2: 8-Bromo-7-fluoro-1-[(1R,3S)-3-hydroxycyclopentyl]-3H-imidazo[4,5-c]quinolin-2-one and 8-bromo-7-fluoro-1-[(1S,3R)-3-hydroxycyclopentyl]-3H-imidazo[4,5-c]quinolin-2-one (1:1 Mixture)

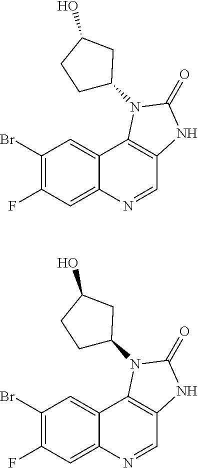 [SMILES: O=c1[nH]c2cnc3cc(F)c(Br)cc3c2n1[C@@H]1CC[C@H](O)C1 ; O=c1[nH]c2cnc3cc(F)c(Br)cc3c2n1[C@H]1CC[C@@H](O)C1]

A mixture of triethylamine (2.105 mL, 15.10 mmol) and 6-bromo-7-fluoro-4-[[(1R,3S)-3-hydroxycyclopentyl]amino]quinoline-3-carboxylic acid: 6-bromo-7-fluoro-4-[[(1S,3R)-3-hydroxycyclopentyl]amino]quinoline-3-carboxylic acid (1:1 mixture) (2 g, 5.03 mmol) in DMF (10 mL) was stirred for 1 h. Diphenyl phosphorazidate (1.663 g, 6.04 mmol) was added and the resulting solution stirred overnight at 60° C. The reaction mixture was poured into water, the solids collected by filtration and dried to afford the desired material as a yellow solid (1.3 g, 71%). NMR Spectrum: ¹H NMR (400 MHz, DMSO-d6) δ 1.88 (2H, dt), 1.97-2.10 (1H, m), 2.17 (1H, m), 2.38 (2H, m), 4.23-4.30 (1H, m), 5.27 (1H, m), 7.88 (1H, m), 8.69 (1H, s), 8.80 (1H, d), 11.77 (1H, s). Mass Spectrum: m/z (ES+)[M+H]+=366.

### Intermediate X3: 6-bromo-7-fluoro-4-[[(1R,3S)-3-hydroxycyclopentyl]amino]quinoline-3-carboxylic acid and 6-bromo-7-fluoro-4-[[(1S,3R)-3-hydroxycyclopentyl]amino]quinoline-3-carboxylic acid (1:1 Mixture)

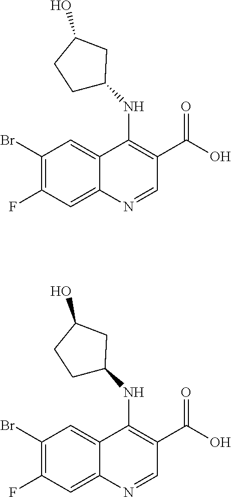 [SMILES: O=C(O)c1cnc2cc(F)c(Br)cc2c1N[C@@H]1CC[C@H](O)C1 ; O=C(O)c1cnc2cc(F)c(Br)cc2c1N[C@H]1CC[C@@H](O)C1]

A mixture of ethyl 6-bromo-7-fluoro-4-[[(1R,3S)-3-hydroxycyclopentyl]amino]quinoline-3-carboxylate: ethyl 6-bromo-7-fluoro-4-[[(1S,3R)-3-hydroxycyclopentyl]amino]quinoline-3-carboxylate (1:1 mixture) (3 g, 7.55 mmol) and sodium hydroxide (0.604 g, 15.10 mmol) in THF (10 mL) and water (5 mL) was stirred for 16 h at 60° C. The organics were removed in vacuo and the pH of the resultant mixture adjusted to 6-7 with 2M HCl. The resultant solid collected by filtration and dried to afford the desired material as a grey solid (2.0 g, 72%). NMR Spectrum: ¹H NMR (400 MHz, DMSO-d6) δ 1.68-1.82 (3H, m), 1.90-1.98 (1H, m), 2.26 (2H, m), 2.51 (4H, s), 4.26 (1H, s), 4.68 (1H, s), 7.86 (1H, d), 8.62 (1H, d), 8.93 (1H, s), 10.95 (1H, s). Mass Spectrum: m/z (ES+)[M+H]+=369.

### Intermediate X4: Ethyl 6-bromo-7-fluoro-4-[[(1R,3S)-3-hydroxycyclopentyl]amino]quinoline-3-carboxylate and ethyl 6-bromo-7-fluoro-4-[[(1S,3R)-3-hydroxycyclopentyl]amino]quinoline-3-carboxylate (1:1 Mixture)

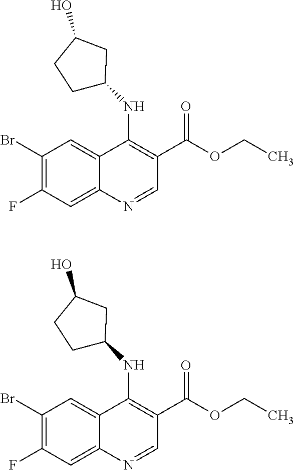 [SMILES: CCOC(=O)c1cnc2cc(F)c(Br)cc2c1N[C@@H]1CC[C@H](O)C1 ; CCOC(=O)c1cnc2cc(F)c(Br)cc2c1N[C@H]1CC[C@@H](O)C1]

DIPEA (3.94 mL, 22.55 mmol) was added to a mixture of cis-3-aminocyclopentanol hydrochloride (1.49 g, 10.83 mmol) and ethyl 6-bromo-4-chloro-7-fluoroquinoline-3-carboxylate (3 g, 9.02 mmol) in DMA (20 mL) under nitrogen and the resulting mixture stirred at 100° C. for 6 h. The reaction mixture was poured into water (50 mL) and the solid filtered and dried to afford the desired material as brown oil (3.0 g, 84%). NMR Spectrum: ¹H NMR (400 MHz, DMSO-d6) δ 1.35 (3H, t), 1.67 (1H, d), 1.72-1.79 (2H, m), 1.81-1.92 (1H, m), 1.96 (3H, s), 2.19 (2H, ddt), 2.79 (3H, s), 2.95 (3H, s), 3.08 (1H, d), 4.23 (1H, s), 4.33 (2H, q), 4.45 (1H, s), 4.83 (1H, s), 7.69 (1H, dd), 8.52 (1H, d), 8.85 (1H, s), 9.25 (1H, d). Mass Spectrum: m/z (ES+)[M+H]+=397.

### Example 45

### 8-[4-[3-(Dimethylamino)propoxy]phenyl]-7-fluoro-3-methyl-1-tetrahydropyran-4-yl-imidazo[4,5-c]quinolin-2-one

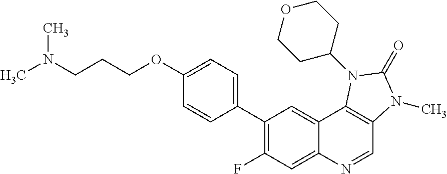

Methanesufonyl chloride (0.136 mL, 1.74 mmol) was added to a solution of 3-(dimethylamino)propan-1-ol (0.172 mL, 1.45 mmol) in DCM (2 mL) at 0° C., over a period of 3 h. The reaction mixture was evaporated to dryness afford crude 3-(dimethylamino)propyl methanesulfonate (264 mg) which was then dissolved in 1,4-dioxane (5 mL) and added in one portion to a stirred suspension of 7-fluoro-8-(4-hydroxyphenyl)-3-methyl-1-tetrahydropyran-4-yl-imidazo[4,5-c]quinolin-2-one (860 mg, 2.18 mmol), and cesium carbonate (949 mg, 2.91 mmol) in 1,4-dioxane (5 mL). The resultant mixture was stirred at 60° C. for 16 h then at 100° C. for a further 2 h. The reaction mixture was evaporated to dryness and re-dissolved in DCM (25 mL), and washed with water. The organic layer was dried over a phase separating cartridge and evaporated to afford crude product which was purified by FCC, elution gradient 0 to 10% MeOH in DCM, to afford the desired material as a white solid (217 mg, 31.1%). NMR Spectrum: ¹H NMR (500 MHz, CDCl₃) δ 1.82-1.97 (2H, m), 2.01 (2H, p), 2.29 (6H, s), 2.50 (2H, t), 2.95 (2H, d), 3.58 (5H, d), 4.11 (2H, t), 4.22 (2H, dd), 5.02 (1H, s), 7.07 (2H, d), 7.61 (2H, d), 7.87 (1H, d), 8.27 (1H, s), 8.68 (1H, s). Mass Spectrum: m/z (ES+)[M+H]+=479

The compound could also be isolated as the methanesulfonic acid salt by dissolving the material (31 mg, 0.06 mmol) in DCM (2 mL) and treating with 1M methanesulfonic acid in DCM (0.07 mL, 0.07 mmol) and then removing the solvent in vacuo. NMR Spectrum: ¹H NMR (500 MHz, DMSO-d6) δ 1.92 (2H, d), 2.17 (2H, dq), 2.33 (3H, s), 2.70 (2H, qd), 2.86 (6H, s), 3.29 (2H, d), 3.52 (5H, s), 4.06 (2H, dd), 4.16 (2H, t), 5.07 (1H, ddd), 7.12-7.19 (2H, m), 7.72 (2H, dd), 7.92 (1H, d), 8.31 (1H, d), 8.94 (1H, s), 9.41 (1H, s).

The following compound was prepared in an analogous fashion from the appropriate alcohol.

| | Example | Structure | Name |
|---|---|---|---|
| AZ13794296 | 46* | 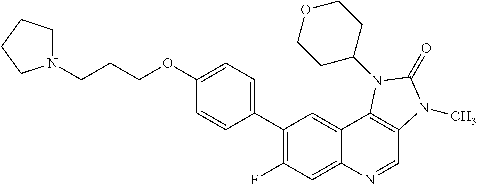 [SMILES: Cn1c(=O)n(C2CCOCC2)c2c3cc(-c4ccc(OCCCN5CCCC5)cc4)c(F)cc3ncc21] | 7-fluoro-3-methyl- 8-[4-(3-pyrrolidin- 1- ylpropoxy)phenyl]- 1-tetrahydropyran- 4-yl-imidazo[4,5- c]quinolin-2-one |

*The reaction was stirred at 100° C. for 2 h and the material purified twice by flash column chromatography and once with an SCX column, eluting with (1M NH₃ in MeOH) in DCM. The material was also isolated as a methanesulfonic acid salt.

### Example 46

(Free base) NMR Spectrum: ¹H NMR (500 MHz, CDCl₃) δ 1.81 (4H, p), 1.93 (2H, d), 2.06 (2H, dt), 2.55 (4H, s), 2.66 (2H, t), 2.95 (2H, d), 3.59 (5H, s), 4.13 (2H, t), 4.22 (2H, dd), 5.02 (1H, s), 7.03-7.1 (2H, m), 7.61 (2H, d), 7.87 (1H, d), 8.28 (1H, s), 8.69 (1H, s). (Methane sulfonic acid salt) NMR Spectrum: ¹H NMR (500 MHz, DMSO-d6) δ 1.92 (5H, d), 2.11-2.22 (2H, m), 2.31 (3H, s), 2.6-2.8 (2H, m), 3.43-3.61 (5H, m), 4.06 (2H, dd), 4.17 (2H, t), 4.94-5.25 (1H, m), 7.15 (2H, d), 7.72 (2H, dd), 7.91 (1H, d), 8.30 (1H, d), 8.92 (1H, s). Mass Spectrum: m/z (ES+)[M+H]+=505

The preparation of 7-fluoro-8-(4-hydroxyphenyl)-3-methyl-1-tetrahydropyran-4-yl-imidazo[4,5-c]quinolin-2-one is described below:

### 7-fluoro-8-(4-hydroxyphenyl)-3-methyl-1-tetrahydropyran-4-yl-imidazo[4,5-c]quinolin-2-one

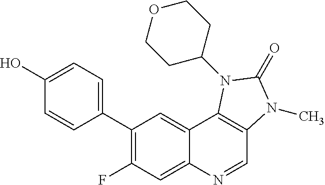 [SMILES: Cn1c(=O)n(C2CCOCC2)c2c3cc(-c4ccc(O)cc4)c(F)cc3ncc21]

Dichlorobis(triphenylphosphine)palladium(II) (18 mg, 0.03 mmol) was added to a mixture of Na₂CO₃ (15.78 mL, 15.78 mmol), 8-bromo-7-fluoro-3-methyl-1-(oxan-4-yl)imidazo[5,4-c]quinolin-2-one (2 g, 5.26 mmol) and (4-hydroxyphenyl)boronic acid (0.871 g, 6.31 mmol) in dioxane (3.6 mL) and the reaction was heated to 100° C. for 16 h.

The reaction was cooled to ambient temperature and filtered under vacuum. The solid was triturated with Et₂O to afford the desired material as a grey solid (1.90 g, 92%). NMR Spectrum: 1H NMR (500 MHz, DMSO-d6) δ 1.90 (2H, d), 2.69 (2H, tt), 3.50 (5H, d), 4.05 (2H, dd), 5-5.09 (1H, m), 6.90 (2H, d), 7.55 (2H, dd), 7.86 (1H, d), 8.26 (1H, d), 8.88 (1H, s). Mass Spectrum: m/z (ES+)[M+H]+=394

The preparation of 8-bromo-7-fluoro-3-methyl-1-(oxan-4-yl)imidazo[5,4-c]quinolin-2-one has been described previously.

### Example 47

### 8-[4-[2-(Dimethylamino)ethoxy]phenyl]-3-methyl-1-[(3S)-tetrahydropyran-3-yl]imidazo[4,5-c]quinolin-2-one

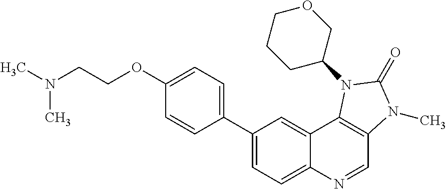

Dichlorobis(di-tert-butyl(3-sulfopropyl)phosphonio)palladate(II) (0.05M in water) (1.132 ml, 0.06 mmol) was added to a degassed mixture of N,N-dimethyl-2-(4-(4,4,5,5-tetramethyl-1,3,2-dioxaborolan-2-yl)phenoxy)ethanamine (0.330 g, 1.13 mmol), 8-bromo-3-methyl-1-[(3S)-oxan-3-yl]imidazo[5,4-c]quinolin-2-one (0.41 g, 1.13 mmol) and 2M K₂CO₃ solution (1.698 ml, 3.40 mmol) in 1,4-dioxane (3.77 mL) and water (0.943 mL) and the reaction heated to 80° C. for 2 h. The reaction mixture was evaporated to dryness, re-dissolved in DCM (100 mL), washed with water (75 mL) and the organic layer dried with a phase separating cartridge and evaporated to afford crude product. The crude product was purified by FCC, elution gradient 0 to 10% MeOH in DCM, to afford the desired material as a white solid (0.410 g, 81%). NMR Spectrum: ¹H NMR (500 MHz, CDCl₃) δ 1.93 (2H, dd), 2.15-2.28 (1H, m), 2.37 (6H, s), 2.72-2.85 (3H, m), 3.56 (4H, s), 4.01-4.07 (1H, m), 4.13-4.23 (3H, m), 4.55 (1H, t), 4.88-5.12 (1H, m), 7.05-7.12 (2H, m), 7.61-7.68 (2H, m), 7.85 (1H, dd), 8.19 (1H, d), 8.32 (1H, s), 8.67 (1H, s). Mass Spectrum: m/z (ES+)[M+H]+=447.

The material was also isolated as the methanesulfonic acid salt by dissolving the material (130 mg, 0.29 mmol) in DCM then adding methanesulfonic acid (0.020 mL, 0.31 mmol) (29 mg in 1 mL of DCM). Et₂O (1 mL) was subsequently added and solvent removed under reduced pressure and dried in a vacuum oven for 2 days. NMR Spectrum: ¹H NMR (500 MHz, DMSO-d6) δ 1.84 (2H, s), 2.17 (1H, d), 2.29 (3H, s), 2.59-2.7 (1H, m), 2.89 (6H, s), 3.37-3.46 (1H, m), 3.49 (3H, s), 3.53-3.6 (2H, m), 3.92 (1H, d), 4.13 (1H, d), 4.24 (1H, t), 4.38-4.44 (2H, m), 4.81-5.09 (1H, m), 7.18-7.24 (2H, m), 7.77-7.83 (2H, m), 7.93 (1H, d), 8.13 (1H, d), 8.32 (1H, s), 8.88 (1H, s), 9.53 (1H, s). Mass Spectrum: m/z (ES+)[M+H]+=447.

The following compounds were prepared in an analogous fashion from the appropriate boronic acid and bromo intermediate, purified by appropriate chromatographic techniques and isolated as either the free base or methanesulfonic acid salt.

| Example | Structure | Name |
|---|---|---|
| 48 | 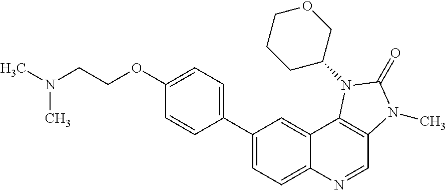 | 8-[4-[2- (dimethylamino)ethoxy]phenyl]- 3-methyl-1-[(3R)- tetrahydropyran-3- yl]imidazo[4,5-c]quinolin-2-one |
| 49* | 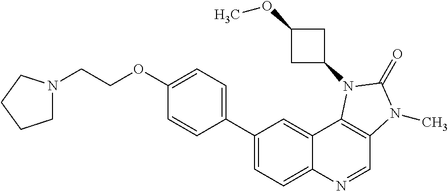 [SMILES: CO[C@H]1C[C@@H](n2c(=O)n(C)c3cnc4ccc(-c5ccc(OCCN6CCCC6)cc5)cc4c32)C1] | 1-(3-(cis)methoxycyclobutyl)-3- methyl-8-[4-(2-pyrrolidin-1- ylethoxy)phenyl]imidazo[4,5- c]quinolin-2-one |
| 50* | 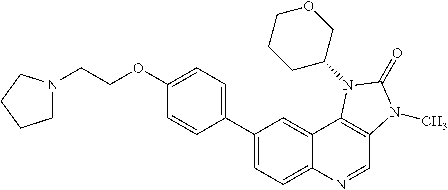 [SMILES: Cn1c(=O)n([C@@H]2CCCOC2)c2c3cc(-c4ccc(OCCN5CCCC5)cc4)ccc3ncc21] | 3-methyl-8-[4-(2-pyrrolidin-1- ylethoxy)phenyl]-1-[(3R)- tetrahydropyran-3- yl]imidazo[4,5-c]quinolin-2-one |
| 51* | 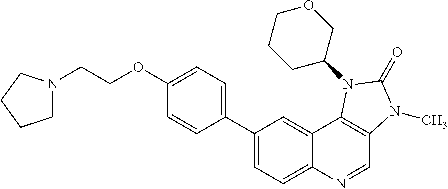 [SMILES: Cn1c(=O)n([C@H]2CCCOC2)c2c3cc(-c4ccc(OCCN5CCCC5)cc4)ccc3ncc21] | 3-methyl-8-[4-(2-pyrrolidin-1- ylethoxy)phenyl]-1-[(3S)- tetrahydropyran-3- yl]imidazo[4,5-c]quinolin-2-one |
| 52 | 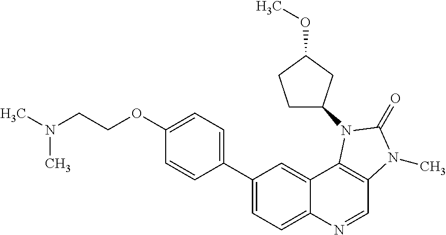 | 8-[4-[2- (dimethylamino)ethoxy]phenyl]- 1-[(1S,3S)-3- methoxycyclopentyl]-3-methyl- imidazo[4,5-c]quinolin-2-one |
| 53** | 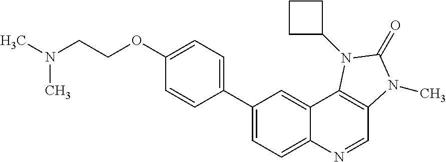 | 1-cyclobutyl-8-[4-[2- (dimethylamino)ethoxy]phenyl]- 3-methyl-imidazo[4,5- c]quinolin-2-one |
| 54 | 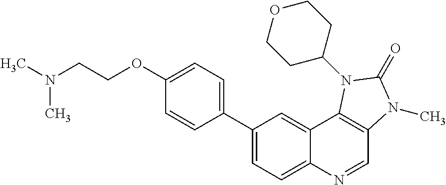 | 8-[4-[2- (dimethylamino)ethoxy]phenyl]- 3-methyl-1-tetrahydropyran-4- yl-imidazo[4,5-c]quinolin-2-one |
| 55 | 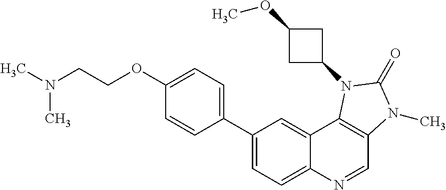 | 8-[4-[2- (dimethylamino)ethoxy]phenyl]- 1-(3-(cis)methoxycyclobutyl)-3- methyl-imidazo[4,5-c]quinolin- 2-one |

*The reaction used chloro(2-dicyclohexylphosphino-2′,4′,6′-triisopropyl-1,1′-biphenyl)[2-(2′-amino-1,1′-biphenyl)]palladium(II) as the catalyst with Cs₂CO₃ as the base and was heated at 80° C. for 4-5 h.
**The reaction used dichlorobis(di-tert-butyl(3-sulfopropyl)phosphonio)palladate(II) (0.05M in water) as the catalyst with K₂CO₃ as the base and was heated at 100° C. for 30 mins.

### Example 48

(Free base) NMR Spectrum: ¹H NMR (500 MHz, CDCl₃) δ 1.89-1.99 (2H, m), 2.17-2.3 (1H, m), 2.37 (6H, s), 2.78 (3H, t), 3.56 (4H, s), 4.01-4.07 (1H, m), 4.13-4.23 (3H, m), 4.54 (1H, t), 5.02 (1H, t), 7.05-7.12 (2H, m), 7.61-7.68 (2H, m), 7.85 (1H, dd), 8.19 (1H, d), 8.32 (1H, s), 8.66 (1H, s). (Methane sulfonic acid salt) NMR Spectrum: ¹H NMR (500 MHz, CDCl₃) δ 1.89-1.97 (2H, m), 2.00 (1H, s), 2.23 (1H, d), 2.38 (6H, s), 2.79 (3H, t), 3.56 (4H, s), 3.96-4.1 (1H, m), 4.13-4.23 (3H, m), 4.55 (1H, t), 4.92-5.14 (1H, m), 7.05-7.12 (2H, m), 7.61-7.68 (2H, m), 7.85 (1H, dd), 8.19 (1H, d), 8.67 (1H, s). Mass Spectrum: m/z (ES+)[M+H]+=447.

### Example 49

NMR Spectrum: 1H NMR (500 MHz, CDCl₃) δ 1.92-1.96 (4H, m), 2.88-3.06 (6H, m), 3.11-3.15 (4H, m), 3.32 (3H.s), 3.57 (3H, s), 3.90-3.95 (1H, m), 4.29 (2H, t), 5.01-5.07 (1H, m), 7.13 (2H, d), 7.74 (2H, d), 7.89 (1H, d), 8.08 (1H, d), 8.33 (1H, s), 8.74 (1H, s). Mass Spectrum: m/z (ES+)[M+H]+=473.

### Example 50

NMR Spectrum: ¹H NMR (300 MHz, MeOH-d4) δ 1.89-1.93 (2H, m), 2.02-2.25 (4H, m), 2.27-2.30 (1H, m), 2.77-2.87 (1H, m), 3.41-3.50 (4H, m), 3.51-3.59 (1H, m), 3.59 (3H, s), 3.65 (2H, t), 3.97-4.05 (1H, m), 4.15-4.25 (1H, m), 4.38-4.45 (3H, m), 5.09-5.19 (1H, m), 7.22 (2H, d), 7.79 (2H, d), 7.97 (1H, d), 8.15 (1H, d), 8.43 (1H, s), 8.80 (1H, s). Mass Spectrum: m/z (ES+)[M+H]+=473.

### Example 51

NMR Spectrum: ¹H NMR (300 MHz, MeOH-d4) δ 1.89-1.93 (6H, m), 2.27-2.30 (1H, m), 2.77-2.87 (5H, m), 3.07 (2H, t), 3.51-3.61 (1H, m), 3.59 (3H, s), 4.0.3-4.07 (1H, m), 4.17-4.32 (3H, m), 4.45 (1H, t), 5.09-5.19 (1H, m), 7.14 (2H, d), 7.74 (2H, d), 7.97 (1H, d), 8.15 (1H, d), 8.43 (1H, s), 8.78 (1H, s). Mass Spectrum: m/z (ES+)[M+H]+=473.

### Example 52

(Free base) NMR Spectrum: ¹H NMR (500 MHz, CDCl₃) δ 1.86-1.99 (1H, m), 2.2-2.35 (3H, m), 2.37 (6H, s), 2.5-2.64 (1H, m), 2.72 (1H, ddd), 2.78 (2H, t), 3.36 (3H, s), 3.58 (3H, s), 4.12-4.21 (3H, m), 5.61 (1H, p), 7.04-7.11 (2H, m), 7.61-7.68 (2H, m), 7.85 (1H, dd), 8.18 (1H, d), 8.34 (1H, d), 8.67 (1H, s). (Methane sulfonic acid salt) NMR Spectrum: ¹H NMR (500 MHz, DMSO-d6) δ 1.82 (1H, s), 2.11-2.26 (3H, m), 2.28 (3H, s), 2.77 (6H, s), 3.27 (3H, s), 3.37 (2H, q), 3.50 (3H, s), 4.03-4.15 (1H, m), 4.35 (2H, d), 5.45-5.65 (1H, m), 7.14-7.2 (2H, m), 7.75-7.81 (2H, m), 7.91 (1H, dd), 8.11 (1H, d), 8.32 (1H, d), 8.87 (1H, s), 9.53 (1H, s). Mass Spectrum: m/z (ES+)[M+H]+=461.

### Example 53

(Methane sulfonic acid salt) NMR Spectrum: ¹H NMR (500 MHz, CDCl₃) δ 1.88-2.01 (1H, m), 2.01-2.12 (1H, m), 2.55 (2H, dddd), 2.83 (3H, s), 2.84 (6H, s), 3.23 (2H, pd), 3.31-3.41 (2H, m), 3.57 (3H, s), 4.4-4.46 (2H, m), 5.31-5.4 (1H, m), 7.07-7.14 (2H, m), 7.61-7.67 (2H, m), 7.79 (1H, dd), 8.16 (1H, d), 8.30 (1H, d), 8.66 (1H, s). Mass Spectrum: m/z (ES+)[M+H]+=417.

### Example 54

(Free base) NMR Spectrum: ¹H NMR (500 MHz, CDCl₃) δ 1.96 (2H, d), 2.37 (6H, s), 2.78 (2H, t), 2.99 (2H, d), 3.60 (5H, s), 4.16 (2H, t), 4.25 (2H, dd), 5.11 (1H, s), 7.06-7.13 (2H, m), 7.68 (2H, d), 7.87 (1H, dd), 8.20 (1H, d), 8.42 (1H, s), 8.69 (1H, s). (Methane sulfonic acid salt) NMR Spectrum: ¹H NMR (500 MHz, DMSO-d6) δ 1.92 (2H, d), 2.28 (3H, s), 2.72 (8H, s), 3.51 (3H, s), 3.56 (2H, t), 4.02-4.14 (2H, m), 4.33 (2H, t), 5.02-5.23 (1H, m), 7.14-7.2 (2H, m), 7.8-7.86 (2H, m), 7.93 (1H, dd), 8.12 (1H, d), 8.41 (1H, s), 8.87 (1H, s), 9.53 (1H, s). Mass Spectrum: m/z (ES+)[M+H]+=447.

### Example 55

(Free base) NMR Spectrum: ¹H NMR (500 MHz, CDCl₃) δ 2.37 (6H, s), 2.79 (2H, t), 2.91-3.02 (2H, m), 3.19 (2H, dddt), 3.31 (3H, s), 3.58 (3H, s), 3.84-3.93 (1H, m), 4.16 (2H, t), 4.93 (1H, tt), 7.05-7.11 (2H, m), 7.62-7.68 (2H, m), 7.83 (1H, dd), 8.18 (1H, d), 8.31 (1H, d), 8.68 (1H, s). (Methane sulfonic acid salt) NMR Spectrum: ¹H NMR (500 MHz, DMSO-d6) δ 2.29 (3H, s), 2.77-2.93 (8H, m), 2.94-3.07 (2H, m), 3.20 (3H, s), 3.56 (4H, d), 3.79-3.96 (2H, m), 4.36-4.48 (2H, m), 5.09-5.27 (1H, m), 7.20 (2H, d), 7.89 (2H, d), 8.14 (1H, s), 8.19 (1H, d), 8.48 (1H, s), 9.13 (1H, s), 9.55 (1H, s). Mass Spectrum: m/z (ES+)[M+H]+=447.

### Example 56

### 8-[4-[2-(Dimethylamino)ethoxy]phenyl]-7-fluoro-3-methyl-1-tetrahydropyran-4-yl-imidazo[4,5-c]quinolin-2-one

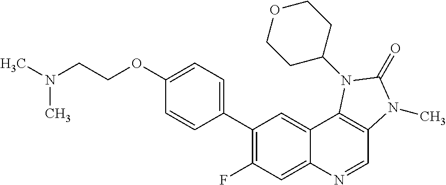

Methanesulfonyl chloride (0.031 mL, 0.40 mmol) was added to 2-(dimethylamino)ethanol (0.034 mL, 0.34 mmol) in DCM (2 mL) at 0° C. and stirred for a period of 2 h under nitrogen. The resulting suspension was evaporated to dryness and the resultant solid added as a suspension to 7-fluoro-8-(4-hydroxyphenyl)-3-methyl-1-tetrahydropyran-4-yl-imidazo[4,5-c]quinolin-2-one (199 mg, 0.50 mmol) and cesium carbonate (202 mg, 0.62 mmol) in 1,4-dioxane (5 mL). The reaction mixture was heated to 100° C. for 16 h then allowed to cool and evaporated to dryness. The residue was re-dissolved in DCM (20 mL), washed with water (20 mL) and the organic layer dried over a phase separating cartridge and evaporated to afford crude product. The crude product was purified by FCC, elution gradient 0 to 10% MeOH in DCM, to afford the desired material as a white solid (65 mg). NMR Spectrum: ¹H NMR (500 MHz, CDCl₃) δ 1.93 (2H, dd), 2.37 (6H, s), 2.78 (2H, t), 2.89-2.98 (2H, m), 3.53-3.61 (5H, m), 4.16 (2H, t), 4.22 (2H, dd), 5.01 (1H, s), 7.05-7.12 (2H, m), 7.61 (2H, dd), 7.87 (1H, d), 8.28 (1H, s), 8.68 (1H, s). Mass Spectrum: m/z (ES+)[M+H]+=465.6.

### BIOLOGICAL ASSAYS

The following assays were used to measure the effects of the compounds of the present invention: a) ATM cellular potency assay; b) PI3K cellular potency assay; c) mTOR cellular potency assay; d) ATR cellular potency assay. During the description of the assays, generally:
i. The following abbreviations have been used: 4NQO=4-Nitroquinoline N-oxide; Ab=Antibody; BSA=Bovine Serum Albumin; CO₂=Carbon Dioxide; DMEM=Dulbecco's Modified Eagle Medium; DMSO=Dimethyl Sulphoxide; EDTA=Ethylenediaminetetraacetic Acid; EGTA=Ethylene Glycol Tetraacetic Acid; ELISA=Enzyme-linked Immunosorbent Assay; EMEM=Eagle's Minimal Essential Medium; FBS=Foetal Bovine Serum; h=Hour(s); HRP=Horseradish Peroxidase; i.p.=intraperitoneal; PBS=Phosphate buffered saline; PBST=Phosphate buffered saline/Tween; TRIS=Tris(Hydroxymethyl)aminomethane; MTS reagent: [3-(4,5-dimethylthiazol-2-yl)-5-(3-carboxymethoxyphenyl)-2-(4-sulfophenyl)-2H-tetrazolium, inner salt, and an electron coupling reagent (phenazine methosulfate) PMS; s.c. sub-cutaneously.
ii. IC₅₀ values were calculated using a smart fitting model in Genedata. The IC₅₀ value was the concentration of test compound that inhibited 50% of biological activity.

### Assay a): ATM Cellular Potency

### Rationale:

Cellular irradiation induces DNA double strand breaks and rapid intermolecular autophosphorylation of serine 1981 that causes dimer dissociation and initiates cellular ATM kinase activity. Most ATM molecules in the cell are rapidly phosphorylated on this site after doses of radiation as low as 0.5 Gy, and binding of a phosphospecific antibody is detectable after the introduction of only a few DNA double-strand breaks in the cell.

The rationale of the p ATM assay is to identify inhibitors of ATM in cells. HT29 cells are incubated with test compounds for 1 hr prior to X-ray-irradiation. 1 h later the cells are fixed and stained for p ATM (Ser1981). The fluorescence is read on the arrayscan imaging platform.

### Method Details:

HT29 cells (ECACC #85061109) were seeded into 384 well assay plates (Costar #3712) at a density of 3500 cells/well in 40 μl EMEM medium containing 1% L glutamine and 10% FBS and allowed to adhere overnight. The following morning compounds of Formula (I) in 100% DMSO were added to assay plates by acoustic dispensing. After 1 h incubation at 37° C. and 5% CO₂, plates (up to 6 at a time) were irradiated using the X-RAD 320 instrument (PXi) with equivalent to ˜600cGy. Plates were returned to the incubator for a further 1 h. Then cells were fixed by adding 20 μl of 3.7% formaldehyde in PBS solution and incubating for 20 minutes at r.t. before being washed with 50 μl/well PBS, using a Biotek EL405 plate washer. Then 20 μl of 0.1% Triton X100 in PBS was added and incubated for 20 minutes at r.t., to permeabalise cells. Then the plates were washed once with 50l/well PBS, using a Biotek EL405 plate washer.

Phospho-ATM Ser1981 antibody (Millipore #MAB3806) was diluted 10000 fold in PBS containing 0.05% polysorbate/Tween and 3% BSA and 20 μl was added to each well and incubated over night at r.t. The next morning plates were washed three times with 50 μl/well PBS, using a Biotek EL405 plate washer, and then 20 μl of secondary Ab solution, containing 500 fold diluted Alexa Fluor® 488 Goat anti-rabbit IgG (Life Technologies, A11001) and 0.002 mg/ml Hoeschst dye (Life technologies #H-3570), in PBS containing 0.05% polysorbate/Tween and 3% BSA, was added. After 1 h incubation at r.t., the plates were washed three times with 50 μl/well PBS, using a Biotek EL405 plate washer, and plates were sealed and kept in PBS at 4° C. until read. Plates were read using an ArrayScan VTI instrument, using an XF53 filter with 10× objective. A two laser set up was used to analyse nuclear staining with Hoeschst (405 nm) and secondary antibody staining of pSer1981 (488 nm).

### Assay b): ATR Cellular Potency

### Rationale:

ATR is a PI 3-kinase-related kinase which phosphorylates multiple substrates on serine or threonine residues in response to DNA damage during or replication blocks. Chk1, a downstream protein kinase of ATR, plays a key role in DNA damage checkpoint control. Activation of Chk1 involves phosphorylation of Ser317 and Ser345 (the latter regarded as the preferential target for phosphorylation/activation by ATR). This was a cell based assay to measure inhibition of ATR kinase, by measuring a decrease in phosphorylation of Chk1 (Ser 345) in HT29 cells, following treatment with compound of Formula (I) and the UV mimetic 4NQO (Sigma #N8141).

### Method Details:

HT29 cells (ECACC #85061109) were seeded into 384 well assay plates (Costar #3712) at a density of 6000 cells/well in 40 μl EMEM medium containing 1% L glutamine and 10% FBS and allowed to adhere overnight. The following morning compound of Formula (I) in 100% DMSO were added to assay plates by acoustic dispensing. After 1 h incubation at 37° C. and 5% CO₂, 40 nl of 3 mM 4NQO in 100% DMSO was added to all wells by acoustic dispensing, except minimum control wells which were left untreated with 4NQO to generate a null response control. Plates were returned to the incubator for a further 1 h. Then cells were fixed by adding 20 μl of 3.7% formaldehyde in PBS solution and incubating for 20 mins at r.t. Then 20 μl of 0.1% Triton X100 in PBS was added and incubated for 10 minutes at r.t., to permeabalise cells. Then the plates were washed once with 50 μl/well PBS, using a Biotek EL405 plate washer.

Phospho-Chk1 Ser 345 antibody (Cell Signalling Technology #2348) was diluted 150 fold in PBS containing 0.05% polysorbate/Tween and 15 μl was added to each well and incubated over night at r.t. The next morning plates were washed three times with 50 μl/well PBS, using a Biotek EL405 plate washer, and then 20 μl of secondary Ab solution, containing 500 fold diluted Alexa Fluor 488 Goat anti-rabbit IgG (Molecular Probes #A-11008) and 0.002 mg/ml Hoeschst dye (Molecular Probes #H-3570), in PBST, was added. After 2 h incubation at r.t., the plates were washed three times with 50 μl/well PBS, using a Biotek EL405 plate washer, and plates were then sealed with black plate seals until read. Plates were read using an ArrayScan VTI instrument, using an XF53 filter with 10× objective. A two laser set up was used to analyse nuclear staining with Hoeschst (405 nm) and secondary antibody staining of pChk1 (488 nm).

### Assay c): PI3K Cellular Potency

### Rationale:

This assay was used to measure PI3K-α inhibition in cells. PDK1 was identified as the upstream activation loop kinase of protein kinase B (Akt1), which is essential for the activation of PKB. Activation of the lipid kinase phosphoinositide 3 kinase (PI3K) is critical for the activation of PKB by PDK1.

Following ligand stimulation of receptor tyrosine kinases, PI3K is activated, which converts PIP2 to PIP3, which is bound by the PH domain of PDK1 resulting in recruitment of PDK1 to the plasma membrane where it phosphorylates AKT at Thr308 in the activation loop.

The aim of this cell-based mode of action assay is to identify compounds that inhibit PDK activity or recruitment of PDK1 to membrane by inhibiting PI3K activity. Phosphorylation of phospho-Akt (T308) in BT474c cells following treatment with compounds for 2 h is a direct measure of PDK1 and indirect measure of PI3K activity.

### Method Details:

BT474 cells (human breast ductal carcinoma, ATCC HTB-20) were seeded into black 384 well plates (Costar, #3712) at a density of 5600 cells/well in DMEM containing 10% FBS and 1% glutamine and allowed to adhere overnight.

The following morning compounds in 100% DMSO were added to assay plates by acoustic dispensing. After a 2 h incubation at 37° C. and 5% CO₂, the medium was aspirated and the cells were lysed with a buffer containing 25 mM Tris, 3 mM EDTA, 3 mM EGTA, 50 mM sodium fluoride, 2 mM Sodium orthovanadate, 0.27M sucrose, 10 mM β-glycerophosphate, 5 mM sodium pyrophosphate, 0.5% Triton X-100 and complete protease inhibitor cocktail tablets (Roche #04 693 116 001, used 1 tab per 50 ml lysis buffer).

After 20 minutes, the cell lysates were transferred into ELISA plates (Greiner #781077) which had been pre-coated with an anti total-AKT antibody in PBS buffer and non-specific binding was blocked with 1% BSA in PBS containing 0.05% Tween 20. Plates were incubated over night at 4° C. The next day the plates were washed with PBS buffer containing 0.05% Tween 20 and further incubated with a mouse monoclonal anti-phospho AKT T308 for 2 h. Plates were washed again as above before addition of a horse anti-mouse-HRP conjugated secondary antibody. Following a 2 h incubation at r.t., plates were washed and QuantaBlu substrate working solution (Thermo Scientific #15169, prepared according to provider's instructions) was added to each well. The developed fluorescent product was stopped after 60 minutes by addition of Stop solution to the wells. Plates were read using a Tecan Safire plate reader using 325 nm excitation and 420 nm emission wavelengths respectively. Except where specified, reagents contained in the Path Scan Phospho AKT (Thr308) sandwich ELISA kit from Cell Signalling (#7144) were used in this ELISA assay.

Assay d): mTOR Cellular Potency

### Rationale:

This assay was used to measure mTOR inhibition in cells. The aim of the phospho-AKT cell based mechanism of action assay using the Acumen Explorer is to identify inhibitors of either PI3Kα or mTOR-Rictor (Rapamycin insensitive companion of mTOR). This is measured by any decrease in the phosphorylation of the Akt protein at Ser473 (AKT lies downstream of PI3Kα in the signal transduction pathway) in the MDA-MB-468 cells following treatment with compound.

### Method Details:

MDA-MB-468 cells (human breast adenocarcinoma #ATCC HTB 132) were seeded at 1500 cells/well in 40 μl of DMEM containing 10% FBS and 1% glutamine into Greiner 384 well black flat-bottomed plates. Cell plates were incubated for 18 h in a 37° C. incubator before dosing with compounds of Formula (I) in 100% DMSO using acoustic dispensing. Compounds were dosed in a 12 point concentration range into a randomised plate map. Control wells were generated either by dosing of 100% DMSO (max signal) or addition of a reference compound (a PI3K-βinhibitor) that completely eliminated the pAKT signal (min control). Plates were incubated at 37° C. for 2 h; cells were then fixed by the addition of 10 μl of a 3.7% formaldehyde solution. After 30 minutes the plates were washed with PBS using a Tecan PW384 plate washer. Wells were blocked and cells permeabilised with the addition of 40 μl of PBS containing 0.5% Tween20 and 1% Marvel™ (dried milk powder) and incubated for 60 minutes at r.t. The plates were washed with PBS containing 0.5% (v/v) Tween20 and 20 μl rabbit anti-phospho AKT Ser473 (Cell Signalling Technologies, #3787) in same PBS-Tween+1% Marvel™ was added and incubated overnight at 4° C.

Plates were washed 3 times with PBS+0.05% Tween 20 using a Tecan PW384. 20 μl of secondary antibody Alexa Fluor 488 anti-Rabbit (Molecular Probes, #A11008) diluted in PBS+0.05% Tween20 containing 1% Marvel™ was added to each well and incubated for 1 h at r.t. Plates were washed three times as before then 20 μl PBS added to each well and plates sealed with a black plate sealer.

The plates were read on an Acumen plate reader as soon as possible, measuring green fluorescence after excitation with 488 nm laser. Using this system IC₅₀ values were generated and quality of plates was determined by control wells. Reference compounds were run each time to monitor assay performance.

TABLE 2
Potency Data for Examples 1-56 in Assays a)-d)

| | Assay a) | Assay b) | Assay c) | Assay d) |
|---|---|---|---|---|
| | ATM Cell | ATR Cell | PI3Kα Cell | mTOR Cell |
| Example | IC₅₀ (μM) | IC₅₀ (μM) | IC₅₀ (μM) | IC₅₀ (μM) |
| 1 | 0.00101 | >30 | | |
| 2 | 0.000984 | >30 | | |
| 3 | 0.000173 | >30 | | |
| 4 | 0.00664 | >30 | | 9.12 |
| 5 | 0.0148 | >30 | >26.9 | |
| 6 | 0.000381 | 24.1 | | 0.781 |
| 7 | 0.000518 | >29 | | |
| 8 | 0.000758 | >30 | 0.311 | 1.42 |
| 9 | 0.0012 | >30 | 15.2 | |
| 10 | 0.00392 | >30 | | |
| 11 | 0.000647 | >30 | | 6.36 |
| 12 | 0.00188 | >30 | 20.4 | 5.56 |
| 13 | 0.00163 | >30 | | |
| 14 | 0.00137 | >29.4 | | |
| 15 | 0.000911 | 21.6 | | 2.58 |
| 16 | 0.000797 | >26 | | |
| 17 | 0.000915 | >25 | | |
| 18 | 0.00105 | >17.8 | | 2.65 |
| 19 | 0.00029 | 18 | | 0.634 |
| 20 | 0.000452 | 21.3 | | 5.5 |
| 21 | 0.000533 | >30 | | 5.07 |
| 22 | 0.000458 | >10 | | |
| 23 | 0.00142 | >30 | | 5.68 |
| 24 | 0.000917 | >24.6 | | 5.91 |
| 25 | 0.000089 | 21 | | 0.879 |
| 26 | 0.000467 | >30 | | |
| 27 | 0.000492 | >18.5 | | |
| 28 | 0.000285 | 11.3 | | |
| 29 | 0.00164 | 12.8 | | |
| 30 | 0.00162 | 15.5 | | 1.7 |
| 31 | 0.000706 | 10.9 | | |
| 32 | 0.00237 | >30 | | |
| 33 | 0.0023 | 28 | | 1.23 |
| 34 | 0.00178 | >10 | | 4.66 |
| 35 | 0.00291 | >30 | | |
| 36 | 0.00577 | >30 | | |
| 37 | 0.00295 | >30 | | 4.11 |
| 38 | 0.00229 | 19.2 | | |
| 39 | | | | 0.899 |
| 40 | 0.000349 | 18.5 | | |
| 41 | 0.00023 | >28.8 | | |
| 42 | 0.000689 | >30 | | 2.17 |
| 43 | 0.000254 | >30 | | 3.63 |
| 44 | 0.000364 | >21.7 | | 6.99 |
| 45 | 0.00395 | 20 | | 8.65 |
| 46 | 0.002 | 16.8 | | |
| 47 | 0.0144 | >30 | >30 | 5.53 |
| 48 | 0.0247 | >26.8 | | 1.78 |
| 49 | 0.0361 | >30 | | 30 |
| 50 | 0.0134 | >25.5 | | 30 |
| 51 | 0.0102 | >30 | | 1.76 |
| 52 | 0.00357 | 21.9 | | 5.63 |
| 53 | 0.00938 | >30 | | 10 |
| 54 | 0.0111 | >24.7 | | 7.73 |
| 55 | 0.0113 | >30 | | 4.44 |
| 56 | 0.0531 | 9.85 | | 29.2 |

Table 3 shows comparative data for certain Compounds of CN102399218A and CN102372711A in tests a) b) c) and d).

TABLE 3
Potency Data for Certain Compounds of CN102399218A
and CN102372711A in Assays a)-d)

| | Assay a) | Assay b) | Assay c) | Assay d) |
|---|---|---|---|---|
| Reference | ATM Cell | ATR Cell | PI3Ka Cell | mTOR Cell |
| Compound | IC₅₀ (μM) | IC₅₀ (μM) | IC₅₀ (μM) | IC₅₀ (μM) |
| CN102372711A | 0.125 | 0.281 | 0.188 | 0.237 |
| Compound 1 | | | | |
| CN102372711A | 0.0112 | 0.0686 | 0.102 | 0.0729 |
| Compound 4 | | | | |
| CN102372711A | 0.0265 | 0.0644 | 0.153 | 0.113 |
| Compound 5 | | | | |
| CN102399218A | 1.76 | >0.0771 | 4.67 | 2.31 |
| Compound 60 | | | | |
| CN102399218A | 3.46 | 1.48 | 1.73 | 0.177 |
| Compound 61 | | | | |
| CN102399218A | 0.08 | 0.0563 | 0.149 | 0.0155 |
| Compound 62 | | | | |
| CN102399218A | 0.216 | 0.162 | 0.247 | 0.287 |
| Compound 64 | | | | |
| CN102399218A | 0.494 | 0.0129 | 0.0804 | 0.0414 |
| Compound 94 | | | | |
| CN102399218A | 0.0741 | 0.0686 | 0.0131 | 0.0469 |
| Compound 114 | | | | |

1. A compound of Formula (I): 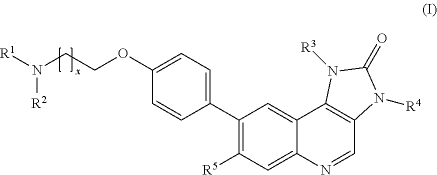
or a pharmaceutically acceptable salt thereof, where:
R¹ is methyl;
R² is hydro or methyl; or
R¹ and R² together with the nitrogen atom to which they are bonded form an azetidinyl, pyrrolidinyl or piperidinyl ring;
x is 1 or 2;
R³ is:
C₄-C₆ cycloalkyl optionally substituted with one methoxy group,
isopropyl,
tetrahydrofuranyl, or
tetrahydropyranyl;
R⁴ is hydro or methyl; and
R⁵ is hydro or fluoro.

2. The compound of Formula (I), or a pharmaceutically acceptable salt thereof, as claimed in claim 1, where R¹ and R² are both methyl; or R¹ and R² together with the nitrogen atom to which they are bonded form an azetidinyl, pyrrolidinyl or piperidinyl ring.

3. The compound of Formula (I), or a pharmaceutically acceptable salt thereof, as claimed in claim 1, where R¹ and R² together with the nitrogen atom to which they are bonded form an azetidinyl, pyrrolidinyl or piperidinyl ring.

4. The compound of Formula (I), or a pharmaceutically acceptable salt thereof, as claimed in claim 1, where R³ is isopropyl, cyclobutyl, 3-methoxycyclobut-1-yl, 3-methoxycyclopent-1-yl, 3-methoxycyclohex-1-yl, 4-methoxycyclohex-1-yl, tetrahydrofuran-3-yl, tetrahydropyran-3-yl or tetrahydropyran-4-yl.

5. The compound of Formula (I), or a pharmaceutically acceptable salt thereof, as claimed in claim 1, where R⁴ is methyl.

6. The compound of Formula (I), or a pharmaceutically acceptable salt thereof as claimed in claim 1, where R⁵ is hydro.

7. The compound of Formula (I), or a pharmaceutically acceptable salt thereof, as claimed in claim 1, where:
R¹ and R² are both methyl; or R¹ and R² together with the nitrogen atom to which they are bonded form an azetidinyl, pyrrolidinyl or piperidinyl ring;
x is 1 or 2;
R³ is isopropyl, cyclobutyl, 3-methoxycyclobut-1-yl, 3-methoxycyclopent-1-yl, 3-methoxycyclohex-1-yl, 4-methoxycyclohex-1-yl, tetrahydrofuran-3-yl, tetrahydropyran-3-yl or tetrahydropyran-4-yl;
R⁴ is methyl; and
R⁵ is hydro or fluoro.

8. The compound of Formula (I), or a pharmaceutically acceptable salt thereof, as claimed in claim 1, where the compound is selected from the group consisting of:
8-[4-[3-(Dimethylamino)propoxy]phenyl]-1-isopropyl-3-methyl-imidazo[4,5-c]quinolin-2-one;
1-Isopropyl-3-methyl-8-[4-(3-pyrrolidin-1-ylpropoxy)phenyl]imidazo[4,5-c]quinolin-2-one;
8-[4-[3-(Azetidin-1-yl)propoxy]phenyl]-1-isopropyl-3-methyl-imidazo[4,5-c]quinolin-2-one;
8-[4-[3-(Azetidin-1-yl)propoxy]phenyl]-7-fluoro-1-isopropyl-3-methyl-imidazo[4,5-c]quinolin-2-one;
8-[4-[2-(Dimethylamino)ethoxy]phenyl]-1-isopropyl-3-methyl-imidazo[4,5-c]quinolin-2-one;
8-[4-[3-(Dimethylamino)propoxy]phenyl]-1-[(1S,3S)-3-methoxycyclopentyl]-3-methyl-imidazo[4,5-c]quinolin-2-one;
8-[4-[3-(Dimethylamino)propoxy]phenyl]-1-[(1R,3R)-3-methoxycyclopentyl]-3-methyl-imidazo[4,5-c]quinolin-2-one;
8-[4-[3-(Dimethylamino)propoxy]phenyl]-3-methyl-1-[(3R)-tetrahydropyran-3-yl]imidazo[4,5-c]quinolin-2-one;
8-[4-[3-(Dimethylamino)propoxy]phenyl]-3-methyl-1-[(3S)-tetrahydropyran-3-yl]imidazo[4,5-c]quinolin-2-one;
8-[4-[3-(Dimethylamino)propoxy]phenyl]-7-fluoro-3-methyl-1-[(3S)-tetrahydropyran-3-yl]imidazo[4,5-c]quinolin-2-one;
8-[4-[3-(Dimethylamino)propoxy]phenyl]-1-(cis-3-methoxycyclobutyl)-3-methyl-imidazo[4,5-c]quinolin-2-one;
8-[4-[3-(Dimethylamino)propoxy]phenyl]-7-fluoro-1-(cis-3-methoxycyclobutyl)-3-methyl-imidazo[4,5-c]quinolin-2-one;
8-[4-[3-(Dimethylamino)propoxy]phenyl]-7-fluoro-1-[trans-3-methoxycyclopentyl]-3-methyl-imidazo[4,5-c]quinolin-2-one;
3-Methyl-8-[4-(3-pyrrolidin-1-ylpropoxy)phenyl]-1-[(3S)-tetrahydropyran-3-yl]imidazo[4,5-c]quinolin-2-one;
3-Methyl-8-[4-(3-pyrrolidin-1-ylpropoxy)phenyl]-1-[(3R)-tetrahydropyran-3-yl]imidazo[4,5-c]quinolin-2-one;
3-Methyl-8-[4-[3-(1-piperidyl)propoxy]phenyl]-1-[(3S)-tetrahydrofuran-3-yl]imidazo[4,5-c]quinolin-2-one;
3-Methyl-8-[4-[3-(1-piperidyl)propoxy]phenyl]-1-tetrahydropyran-4-yl-imidazo[4,5-c]quinolin-2-one;
1-[trans-3-Methoxycyclopentyl]-3-methyl-8-[4-(3-pyrrolidin-1-ylpropoxy)phenyl]imidazo[4,5-c]quinolin-2-one;
8-[4-[3-(Dimethylamino)propoxy]phenyl]-1-(trans-3-methoxycyclobutyl)-3-methyl-imidazo[4,5-c]quinolin-2-one;
1-(trans-4-Methoxycyclohexyl)-3-methyl-8-[4-(3-pyrrolidin-1-ylpropoxy)phenyl]imidazo[4,5-c]quinolin-2-one;
8-[4-[3-(Azetidin-1-yl)propoxy]phenyl]-7-fluoro-1-[trans-3-methoxycyclopentyl]-3-methyl-imidazo[4,5-c]quinolin-2-one;
8-[4-[3-(Dimethylamino)propoxy]phenyl]-1-(cis-4-methoxycyclohexyl)-3-methyl-imidazo[4,5-c]quinolin-2-one;
8-[4-[3-(Dimethylamino)propoxy]phenyl]-1-(cis-4-methoxycyclohexyl)-3-methyl-imidazo[4,5-c]quinolin-2-one;
1-(cis-4-Methoxycyclohexyl)-3-methyl-8-[4-(3-pyrrolidin-1-ylpropoxy)phenyl]imidazo[4,5-c]quinolin-2-one;
8-[4-[3-(Dimethylamino)propoxy]phenyl]-1-[trans-3-methoxycyclohexyl]-3-methyl-imidazo[4,5-c]quinolin-2-one;
8-[4-[3-(Dimethylamino)propoxy]phenyl]-1-[trans-3-methoxycyclohexyl]-3-methyl-imidazo[4,5-c]quinolin-2-one;
1-[trans-3-Methoxycyclohexyl]-3-methyl-8-[4-(3-pyrrolidin-1-ylpropoxy)phenyl]imidazo[4,5-c]quinolin-2-one;
8-[4-[3-(Dimethylamino)propoxy]phenyl]-1-[cis-3-methoxycyclohexyl]-3-methyl-imidazo[4,5-c]quinolin-2-one;
8-[4-[3-(Dimethylamino)propoxy]phenyl]-1-[cis-3-methoxycyclohexyl]-3-methyl-imidazo[4,5-c]quinolin-2-one;
8-[4-[3-(Dimethylamino)propoxy]phenyl]-1-[cis-3-methoxycyclopentyl]-3-methyl-imidazo[4,5-c]quinolin-2-one;
8-[4-[3-(Dimethylamino)propoxy]phenyl]-7-fluoro-1-[cis-3-methoxycyclopentyl]-3-methyl-imidazo[4,5-c]quinolin-2-one;
1-[cis-3-Methoxycyclohexyl]-3-methyl-8-[4-(3-pyrrolidin-1-ylpropoxy)phenyl]imidazo[4,5-c]quinolin-2-one;
1-[cis-3-Methoxycyclohexyl]-3-methyl-8-[4-(3-pyrrolidin-1-ylpropoxy)phenyl]imidazo[4,5-c]quinolin-2-one;
1-[(1S,3S)-3-Methoxycyclopentyl]-3-methyl-8-[4-[3-(1-piperidyl)propoxy]phenyl]imidazo[4,5-c]quinolin-2-one;
1-(cis-3-Methoxycyclobutyl)-3-methyl-8-[4-(3-pyrrolidin-1-ylpropoxy)phenyl]imidazo[4,5-c]quinolin-2-one;
1-(trans-3-Methoxycyclobutyl)-3-methyl-8-[4-(3-pyrrolidin-1-ylpropoxy)phenyl]imidazo[4,5-c]quinolin-2-one;
1-(cis-3-Methoxycyclobutyl)-3-methyl-8-[4-[3-(1-piperidyl)propoxy]phenyl]imidazo[4,5-c]quinolin-2-one;
8-[4-[3-(Dimethylamino)propoxy]phenyl]-3-methyl-1-tetrahydropyran-4-yl-imidazo[4,5-c]quinolin-2-one;
8-[4-[3-(Dimethylamino)propoxy]phenyl]-7-fluoro-3-methyl-1-tetrahydropyran-4-yl-imidazo[4,5-c]quinolin-2-one;
7-Fluoro-3-methyl-8-[4-(3-pyrrolidin-1-ylpropoxy)phenyl]-1-tetrahydropyran-4-yl-imidazo[4,5-c]quinolin-2-one;
8-[4-[2-(Dimethylamino)ethoxy]phenyl]-3-methyl-i-[(3S)-tetrahydropyran-3-yl]imidazo[4,5-c]quinolin-2-one;
8-[4-[2-(Dimethylamino)ethoxy]phenyl]-3-methyl-1-[(3R)-tetrahydropyran-3-yl]imidazo[4,5-c]quinolin-2-one;
1-(3-(cis)Methoxycyclobutyl)-3-methyl-8-[4-(2-pyrrolidin-1-ylethoxy)phenyl]imidazo[4,5-c]quinolin-2-one;
3-Methyl-8-[4-(2-pyrrolidin-1-ylethoxy)phenyl]-1-[(3R)-tetrahydropyran-3-yl]imidazo[4,5-c]quinolin-2-one;
3-Methyl-8-[4-(2-pyrrolidin-1-ylethoxy)phenyl]-1-[(3S)-tetrahydropyran-3-yl]imidazo[4,5-c]quinolin-2-one;
8-[4-[2-(Dimethylamino)ethoxy]phenyl]-1-[(1S,3S)-3-methoxycyclopentyl]-3-methyl-imidazo[4,5-c]quinolin-2-one;
1-Cyclobutyl-8-[4-[2-(dimethylamino)ethoxy]phenyl]-3-methyl-imidazo[4,5-c]quinolin-2-one;
8-[4-[2-(Dimethylamino)ethoxy]phenyl]-3-methyl-1-tetrahydropyran-4-yl-imidazo[4,5-c]quinolin-2-one;
8-[4-[2-(Dimethylamino)ethoxy]phenyl]-1-(3-(cis)methoxycyclobutyl)-3-methyl-imidazo[4,5-c]quinolin-2-one; and
8-[4-[2-(Dimethylamino)ethoxy]phenyl]-7-fluoro-3-methyl-1-tetrahydropyran-4-yl-imidazo[4,5-c]quinolin-2-one.

9. A pharmaceutical composition which comprises a compound of Formula (I), or a pharmaceutically acceptable salt thereof, as claimed in claim 1, and at least one pharmaceutically acceptable excipient.

10.-14. (canceled)

15. A method for treating cancer in a warm-blooded animal in need of such treatment, which comprises administering to said warm-blooded animal a therapeutically effective amount of a compound of Formula (I), or a pharmaceutically acceptable salt thereof, as claimed in claim 1.

16. The method of claim 15, wherein the compound of Formula (I), or a pharmaceutically acceptable salt thereof, is administered simultaneously, separately or sequentially with at least one additional anti-tumour substance selected from cisplatin, oxaliplatin, carboplatin, valrubicin, idarubicin, doxorubicin, pirarubicin, irinotecan, topotecan, amrubicin, epirubicin, etoposide, mitomycin, bendamustine, chlorambucil, cyclophosphamide, ifosfamide, carmustine, melphalan, bleomycin, olaparib, MEDI4736, AZD1775 and AZD6738.
